(19) United States
(12) Patent Application Publication
(10) Pub. No.: US 2025/0388699 A1
(43) Pub. Date: Dec. 25, 2025

(54) VARIANT FC REGIONS

(71) Applicant: argenx BV, Ghent (BE)

(72) Inventors: Michael Saunders, Ghent (BE); René Bigirimana, Ghent (BE); Christophe Blanchetot, Ghent (BE); Marijn Cromheecke, Ghent (BE); Conor McGuire, Ghent (BE)

(21) Appl. No.: 19/257,976

(22) Filed: Jul. 2, 2025

Related U.S. Application Data
(62) Division of application No. 17/598,033, filed on Sep. 24, 2021.

(30) Foreign Application Priority Data
Apr. 11, 2019 (GB) 1905150.7

(51) Int. Cl. C07K 16/42; A61K 39/00; A61K 39/395; A61P 11/06; A61P 37/02

(52) U.S. Cl. CPC C07K 16/4291; A61K 39/39566; A61P 11/06; A61P 37/02; A61K 2039/505; C07K 2317/24; C07K 2317/33; C07K 2317/52; C07K 2317/55; C07K 2317/565; C07K 2317/76; C07K 2317/92

(57) ABSTRACT

The present invention relates to antibodies that bind to IgE and their use in the treatment of autoimmune diseases, particularly Bullous Pemphigoid (BP) and Chronic Spontaneous Urticaria (CSU). The anti-IgE antibodies comprise a variant Fc domain that binds to the Fc receptor FcRn with increased affinity relative to a wild-type Fc domain. The anti-IgE antibodies may comprise a variant Fc domain comprising the amino acids Y, T, E, K, F and Y at EU positions 252, 254, 256, 433, 434 and 436, respectively, wherein the variant Fc domain binds to human FcRn with increased affinity relative to a wild-type human IgG Fc domain.

### RELATED APPLICATIONS

This application is a Divisional of U.S. patent application Ser. No. 17/598,033, filed Sep. 24, 2021, which is a 35 U.S.C. § 371 application of International Patent Application No. PCT/EP2020/060240, filed Apr. 9, 2020, which claims priority to Great Britain Patent Application No. 1905150.7, filed Apr. 11, 2019, the entire disclosures of which are hereby incorporated herein by reference.

### SUBMISSION OF SEQUENCE LISTING XML

The content of the following submission of Sequence Listing XML is incorporated herein by reference in its entirety: a computer readable form (CRF) of the Sequence Listing (file name: 404373-T1901US_SL.xml, date created: Jun. 27, 2025, size: 226,995 bytes).

### FIELD OF THE INVENTION

The present invention relates to antibodies that bind to IgE and their use in the treatment of autoimmune diseases, particularly Bullous Pemphigoid (BP) and Chronic Spontaneous Urticaria (CSU). The anti-IgE antibodies comprise a variant Fc domain that binds to the Fc receptor FcRn with increased affinity relative to a wild-type Fc domain. The anti-IgE antibodies may comprise a variant Fc domain incorporating ABDEG™ technology wherein the variant ABDEG™ Fc domain binds to FcRn with increased affinity relative to a wild-type Fc domain. FcRn is important for the plasma recycling of IgG antibodies, including IgG autoantibodies. The anti-IgE antibodies of the invention thus provide dual targeting of IgE and IgG autoantibodies in the treatment of autoimmune diseases.

### BACKGROUND TO THE INVENTION

Immunoglobulin E (IgE) was first discovered in 1966 and is the least abundant of the immunoglobulin classes or isotypes. IgE molecules play a central role in human allergy, primarily by virtue of their high-affinity association with receptors on mast cells and basophils, specifically FcεRI receptors. Allergen binding to IgE molecules causes FcεRI receptor cross-linking, which triggers the release of histamine and other inflammatory mediators from the effector cells in a process termed “degranulation”. IgE-mediated stimulation also leads to the synthesis of numerous cytokines and other factors that produce an inflammatory response. IgE also associates with a low-affinity receptor (FcεRII or CD23) located on cell types including B cells, macrophages and platelets.

Given the central role played by IgE molecules in diseases such as asthma, allergic rhinitis and other allergic disorders, IgE has long been an attractive therapeutic target for these diseases. The challenge in developing an agent, for example an antibody, to target IgE has been to produce an agent that does not itself cross-link IgE-receptor complexes i.e. the agent must be non-anaphylactogenic. In diseases such as asthma and allergic disorders, the triggers for mast cell and basophil degranulation are exogenous ligands of specific IgE antibodies. More recently, it has become apparent that IgE antibodies recognizing autoantigens can also trigger degranulation in response to their cognate ligands. Thus IgEs can play a role in autoimmune diseases such as some forms of Chronic Urticaria (including CSU and CIndU), and Bullous Pemphigoid. Numerous other autoimmune diseases may also involve IgE antibodies recognizing self-antigens (see Maurer et al. Frontiers in Immunology (2018)9: 1-17; and Sanjuan et al. JACI 137(6): 1651-1661).

Omalizumab is a humanized monoclonal anti-IgE antibody with a high binding affinity for IgE (for reviews, see Kawaki et al. J. Immunol. (2016) 197(11): 4187-9192; and Schulman E. S. Am J Respir Crit Care Med. (2001) 164: S6-S11). Omalizumab inhibits allergic responses by binding to serum IgE molecules, thereby preventing the interaction of IgE with IgE receptors. Unlike other anti-IgE antibodies that can cross-link FcεRI-bound IgE, omalizumab does not cause an anaphylactic effect. Omalizumab binds to the Cε3 (or CH3) domain of free IgE preventing it from binding to FcεRI. By depleting serum IgE, omalizumab also down-regulates the expression of FcεRI on mast cells and basophils as well as antigen-presenting cells. This, in turn, makes them less sensitive to degranulation and thus limits the activation of mast cells and basophils. In addition to the depletion of free IgE and downregulation of FcεRI on mast cells and basophils, it has been suggested that omalizumab may exert its therapeutic effects via a variety of other mechanisms.

Omalizumab was first approved in the US and the EU for the treatment of allergic asthma. In 2014, it was approved for use in patients with Chronic Spontaneous Urticaria (CSU). CSU is a highly debilitating skin disease. It is characterized by the presence of itchy wheal-and-flare skin reactions, angioedema, or both, for a period of greater than 6 weeks. The wheal and angioedema observed in CSU appear to involve the degranulation of skin mast cells, which release histamine, proteases, and cytokines together with generation of platelet-activating factor and other arachidonic metabolites. These mediators induce vasodilatation, increase vascular permeability, and stimulate sensory nerve endings that lead to swelling, redness and itch. A lesion site or wheal is characterised by edema, mast cell degranulation, and a perivascular infiltrate of cells—CD4+ lymphocytes, monocytes, neutrophils, eosinophils, and basophils. Around half of patients with CSU can be successfully treated with antihistamines. However, in those for which antihistamines fail, omalizumab is approved as second-line therapy (for reviews, see Ferrer M. Clin Transl Allergy (2015) 5:30; Kolkhir et al. J Allergy Clin Immunol. (2017) 139: 1772-81; Kaplan A. P. Allergy Asthma Immunol Res. (2017) 9(6): 477-482).

A great deal of work has been carried out to elucidate the mechanisms by which omalizumab exerts its therapeutic effect in patients having CSU (see Chang et al. J Allergy Clin Immunol. (2015) 135: 337-42; and Kaplan et al. Allergy (2017) 72(4): 519-533). IgE clearly plays an important role in the pathogenesis of CSU and accumulating evidence has shown that IgE, by binding to FcεRI on mast cells, can promote the proliferation and survival of these cells thereby expanding the mast cell pool. IgE and FcεRI engagement can also decrease the release threshold of mast cells and increase their sensitivity to various stimuli. The reversal of these effects by omalizumab is likely to account, at least in part, for its efficacy in treating CSU.

In addition to the above, it has been observed that CSU has an important autoimmune component. It has in fact been suggested that autoimmune processes might be the primary cause of most cases of CSU. CSU patients frequently exhibit increased total IgE levels and have associated autoimmune conditions, especially thyroid autoimmune disorders such as Hashimoto thyroiditis. Studies have reported the presence in CSU patient sera of autoreactive IgE molecules directed against thyroperoxidase (TPO) and against dsDNA. It is likely therefore, that omalizumab exerts its therapeutic effect, at least in part, by inhibiting autoreactive IgE antibodies.

In addition to CSU, a pathophysiological role of autoreactive IgEs has been observed in several other autoimmune diseases including systemic conditions such as SLE and also tissue-specific diseases such as Grave's disease. One disease in which IgE autoantibodies are thought to play a key role is Bullous Pemphigoid (BP). BP is the most common antibody-mediated autoimmune blistering disease of the skin. The disease occurs mainly in the elderly (median age of presentation in the UK is 80 years) and is characterised by tense bullae and urticarial type plaques. Studies on BP patients have revealed that about 50% of patients have blood eosinophilia and about 70% have elevated serum IgE. In addition, more than 70% of patients have serum IgE against the antigen BP180 (or BPAg2), a type XVII collagen (COL17) protein, which acts as the adhesion molecule between the epidermis and the basement membrane of the dermis. A second autoantigen has also been identified as the target of autoreactive IgE in BP patients. This autoantigen is BP230 (or BP antigen 1 or BPAG1/BPAG1e), a cell adhesion junction plaque protein which localises to the hemidesmosome (see, Hammers et al. Annu. Rev. Pathol. Mech. Dis. (2016) 11: 175-197; Saniklidou et al. Arch Dermatol Res. (2018) 310(1): 11-28). Although not yet authorised for the treatment of BP, omalizumab has proven to be effective in treating the symptoms of BP in some human subjects (Fairley et al. J. Allergy Clin Immunol. (2009) 123: 704-705; Dufour et al. Br J. Dermatol. (2012) 166: 1140-1142; Yu et al. J. Am. Acad.

Dermatol. (2014) 71(3): 468-474).

### SUMMARY OF THE INVENTION

Given the importance of IgE immunoglobulins in both allergic and autoimmune diseases, there is a need to develop improved agents, for example antibodies, that target IgE. The present invention addresses this problem by the provision of novel anti-IgE antibodies.

Furthermore, the present invention seeks to provide anti-IgE antibodies that are particularly suited to the treatment of autoimmune diseases caused by both autoreactive IgE antibodies and autoreactive IgG antibodies. As noted above, CSU and BP are two examples of autoimmune diseases in which autoreactive IgE antibodies play a key role in the pathophysiology. In both of these diseases, autoreactive IgG antibodies against self-antigens have also been identified in some patients.

In CSU, IgG autoantibodies that bind to the high-affinity IgE receptor, FcεRI, have been observed in 35%-40% patients. IgG autoantibodies that bind to IgE itself have also been observed in 5%-10% patients. The cross-linking of FcεRI receptors on mast cells and basophils by the direct binding of anti-FcεRI IgG autoantibodies or via the indirect binding of anti-IgE IgG autoantibodies is likely to play an important role in the pathogenesis of this disease.

BP is also characterised by the presence of IgG autoantibodies, for example IgG autoantibodies that bind to the BP180 antigen described above. IgE autoantibodies against the NC16A domain of BP180 were found in 77% of sera tested and were equivalent to the frequency of anti-BP180 NC16A IgG autoantibodies. Together with the autoreactive anti-BP180 IgE autoantibodies, the anti-BP180 IgG autoantibodies identified in patients having BP are thought to play a causative role in disease progression. IgG autoantibodies bind to BP180 at the basement membrane zone and induce complement activation and recruitment of neutrophils. Neutrophils induce the cleavage of BP180 and cleaved BP180 is linked by IgE autoantibodies leading to the activation of eosinophils and mast cells and worsening of the disease.

Taking into account the above, the present inventors considered the possibility of dual targeting of IgE and IgG autoantibodies as an effective strategy to treat diseases having both an autoreactive IgE and IgG pathogenic component. As reported herein, the antibodies of the invention exhibit binding specificity for IgE and have the ability to deplete IgG levels by binding to the Fc receptor FcRn with higher affinity than native IgG molecules. These antibodies provide a two-pronged approach to the treatment of autoimmune diseases such as BP and CSU.

In a first aspect, the present invention provides an antibody that binds to IgE, wherein the antibody comprises a variant Fc domain or a FcRn binding fragment thereof that binds to FcRn with increased affinity relative to a wild-type Fc domain.

In certain embodiments, the variant Fc domain or FcRn binding fragment thereof binds to FcRn with increased affinity relative to a wild-type IgG Fc domain. In certain embodiments, the variant Fc domain or FcRn binding fragment thereof binds to human FcRn with increased affinity relative to a wild-type human IgG Fc domain. In preferred embodiments, the variant Fc domain or FcRn binding fragment thereof binds to human FcRn with increased affinity relative to a wild-type human IgG1 Fc domain.

In certain embodiments, the variant Fc domain or FcRn binding fragment thereof binds to human FcRn with increased affinity at pH 6.0. In certain embodiments, the variant Fc domain or FcRn binding fragment thereof binds to human FcRn with increased affinity at pH 7.4. In preferred embodiments, the variant Fc domain or FcRn binding fragment thereof binds to human FcRn with increased affinity at pH 6.0 and pH 7.4.

In certain embodiments, the variant Fc domain or FcRn binding fragment thereof binds to human FcRn at pH 6.0 with a binding affinity that is increased by at least 20× as compared with a wild-type human IgG1 Fc domain. In preferred embodiments, the variant Fc domain or FcRn binding fragment thereof binds to human FcRn at pH 6.0 with a binding affinity that is increased by at least 30× as compared with a wild-type human IgG1 Fc domain.

In certain embodiments, the binding affinity of the variant Fc domain or FcRn binding fragment for human FcRn at pH 6.0 is stronger than K_{D} 15 nM. In certain embodiments, the binding affinity of the variant Fc domain or FcRn binding fragment for human FcRn at pH 7.4 is stronger than K_{D} 320 nM.

In certain embodiments, the variant Fc domain or FcRn binding fragment thereof comprises at least one amino acid substitution, at least two amino acid substitutions, at least three amino acid substitutions as compared with the corresponding wild-type Fc domain. The variant Fc domain or FcRn binding fragment thereof may comprise at least one amino acid, at least two amino acids or at least three amino acids selected from the following: 237M; 238A; 239K; 248I; 250A; 250F; 2501; 250M; 250Q; 250S; 250V; 250W; 250Y; 252F; 252W; 252Y; 254T; 255E; 256D; 256E; 256Q; 257A; 257G; 2571; 257L; 257M; 257N; 257S; 257T; 257V; 258H; 265A; 270F; 286A; 286E; 289H; 297A; 298G; 303A; 305A; 307A; 307D; 307F; 307G; 307H; 307I; 307K; 307L; 307M; 307N; 307P; 307Q; 307R; 307S; 307V; 307W; 307Y; 308A; 308F; 3081; 308L; 308M; 308P; 308Q; 308T; 309A; 309D; 309E; 309P; 309R; 311A; 311H; 311I; 312A; 312H; 314K; 314R; 315A; 315H; 317A; 325G; 332V; 334L; 360H; 376A; 378V; 380A; 382A; 384A; 385D; 385H; 386P; 387E; 389A; 389S; 424A; 428A; 428D; 428F; 428G; 428H; 4281; 428K; 428L; 428N; 428P; 428Q; 428S; 428T; 428V; 428W; 428Y; 433K; 434A; 434F; 434H; 434S; 434W; 434Y; 436H; 4361 and 436F, wherein the positions are defined in accordance with EU numbering.

In preferred embodiments, the variant Fc domain or FcRn binding fragment thereof comprises the amino acids Y, T, E, K, F and Y at EU positions 252, 254, 256, 433, 434 and 436, respectively.

The variant Fc domain or FcRn binding fragment thereof may comprise at least one, at least two or at least three amino acid substitution(s) selected from: G237M; P238A; S239K; K248I; T250A; T250F; T2501; T250M; T250Q; T250S; T250V; T250W; T250Y; M252F; M252W; M252Y; S254T; R255E; T256D; T256E; T256Q; P257A; P257G; P2571; P257L; P257M; P257N; P257S; P257T; P257V; E258H; D265A; D270F; N286A; N286E; T289H; N297A; S298G; V303A; V305A; T307A; T307D; T307F; T307G; T307H; T307I; T307K; T307L; T307M; T307N; T307P; T307Q; T307R; T307S; T307V; T307W; T307Y; V308A; V308F; V3081; V308L; V308M; V308P; V308Q; V308T; V309A; V309D; V309E; V309P; V309R; Q311A; Q311H; Q311I; D312A; D312H; L314K; L314R; N315A; N315H; K317A; N325G; 1332V; K334L; K360H; D376A; A378V; E380A; E382A; N384A; G385D; G385H; Q386P; P387E; N389A; N389S; S424A; M428A; M428D; M428F; M428G; M428H; M4281; M428K; M428L; M428N; M428P; M428Q; M428S; M428T; M428V; M428W; M428Y; H433K; N434A; N434F; N434H; N434S; N434W; N434Y; Y436H; Y4361 and Y436F, wherein the positions are defined in accordance with EU numbering.

In preferred embodiments, the variant Fc domain or FcRn binding fragment thereof comprises the amino acid substitutions M252Y, S254T, T256E, H433K and N434F.

In certain embodiments, the variant Fc domain or FcRn binding fragment thereof does not comprise the combination of amino acids Y, P and Y at EU positions 252, 308 and 434, respectively. In certain embodiments, the variant Fc domain or FcRn binding fragment does not comprise the combination of amino acid substitutions: M252Y, V308P and N434Y.

Also provided herein is an antibody that binds to IgE, wherein the antibody comprises a variant Fc domain or a FcRn binding fragment thereof, said variant Fc domain or FcRn binding fragment comprising the amino acids Y, T, E, K, F and Y at EU positions 252, 254, 256, 433, 434 and 436, respectively.

In certain embodiments relating to all anti-IgE antibodies described herein, the variant Fc domain or FcRn binding fragment thereof is a variant human Fc domain or FcRn binding fragment thereof. The variant Fc domain or FcRn binding fragment thereof may be a variant IgG Fc domain or FcRn binding fragment thereof. The variant Fc domain or FcRn binding fragment thereof may be a variant IgG1 Fc domain or FcRn binding fragment thereof, preferably a variant human IgG1 Fc domain or FcRn binding fragment thereof.

In certain embodiments relating to all anti-IgE antibodies described herein, the variant Fc domain or FcRn binding fragment thereof consists of no more than 20, no more than 10 or no more than amino acid substitutions as compared with the corresponding wild-type Fc domain.

In certain preferred embodiments, the variant Fc domain comprises or consists of the amino acid sequence set forth in SEQ ID NO: 1, SEQ ID NO: 2 or SEQ ID NO: 3. In further preferred embodiments, the variant Fc domain comprises or consists of the amino acid sequence set forth in SEQ ID NO: 5, SEQ ID NO: 6 or SEQ ID NO: 7.

In certain embodiments, the variant Fc domain or FcRn binding fragment thereof is comprised within a variant Fc region, said variant Fc region consisting of two Fc domains or FcRn binding fragments thereof. The two Fc domains or FcRn binding fragments of the variant Fc region may be identical. In such embodiments, the two Fc domains of the variant Fc region may each comprise or consist of the amino acid sequence set forth in SEQ ID NO: 1, SEQ ID NO: 2 or SEQ ID NO: 3. Alternatively, the two Fc domains of the variant Fc region may each comprise or consist of the amino acid sequence set forth in SEQ ID NO: 5, SEQ ID NO: 6 or SEQ ID NO: 7.

For embodiments wherein the anti-IgE antibody comprises a variant Fc region, the variant Fc region may have increased affinity for CD16a. In certain embodiments, the Fc domains of the variant Fc region do not comprise an N-linked glycan at EU position 297. Alternatively, the Fc domains of the variant Fc region comprise an afucosylated N-linked glycan at EU position 297. Alternatively, the Fc domains of the variant Fc region comprise an N-linked glycan having a bisecting GlcNac at EU position 297 of the Fc domains.

The anti-IgE antibodies provided herein may bind to the CH3 domain of IgE. Binding to IgE may inhibit binding of IgE to FcεRI and/or inhibit mast cell or basophil degranulation. In preferred embodiments, the anti-IgE antibodies are not anaphylactic.

In certain preferred embodiments, the anti-IgE antibodies exhibit pH-dependent target binding such that the antibody exhibits lower antigen-binding activity at acidic pH than at neutral pH. The ratio of antigen-binding activity at acidic pH and at neutral pH may be at least 2, at least 3, at least 5, at least 10, as measured by KD(at acidic pH)/KD(at neutral pH). In certain embodiments, the pH-dependent anti-IgE antibodies comprise one or more CDRs comprising one or more His substitutions.

The anti-IgE antibodies provided herein may be IgG antibodies, preferably IgG1 antibodies. In certain embodiments, the anti-IgE antibodies are humanised or germlined variants of non-human antibodies, for example camelid-derived antibodies. In certain embodiments, the anti-IgE antibodies comprise the CDR, VH and/or VL sequences of the exemplary anti-IgE antibodies described herein.

Further provided herein are polynucleotides encoding the anti-IgE antibodies, and expression vectors comprising said polynucleotides operably linked to regulatory sequences which permit expression of the antibody. Also provided are host cells or cell-free expression systems containing the expression vectors. Further provided are methods of producing recombinant antibodies, the methods comprising culturing the host cells or cell free expression systems under conditions which permit expression of the antibody and recovering the expressed antibody.

In a further aspect, the present invention provides pharmaceutical compositions comprising an anti-IgE antibody of the invention and at least one pharmaceutically acceptable carrier or excipient. The anti-IgE antibodies and pharmaceutical compositions comprising the same may be for use as medicaments.

In still further aspects, the present invention provides methods of treating antibody-mediated disorders in subjects, preferably human subjects. The methods comprise administering to a patient in need thereof a therapeutically effective amount of an anti-IgE antibody or a pharmaceutical composition according to the aspects of the invention described above.

The antibody-mediated disorder may be an IgE-mediated disorder. Alternatively or in addition, the antibody-mediated disorder may be an autoimmune disease. The autoimmune disease may be selected from the group consisting of allogenic islet graft rejection, alopecia areata, ankylosing spondylitis, antiphospholipid syndrome, autoimmune Addison's disease, Alzheimer's disease, antineutrophil cytoplasmic autoantibodies (ANCA), autoimmune diseases of the adrenal gland, autoimmune hemolytic anemia, autoimmune hepatitis, autoimmune myocarditis, autoimmune neutropenia, autoimmune oophoritis and orchitis, autoimmune thrombocytopenia, autoimmune urticaria, Behcet's disease, bullous pemphigoid, cardiomyopathy, Castleman's syndrome, celiac spruce-dermatitis, chronic fatigue immune disfunction syndrome, chronic inflammatory demyelinating polyneuropathy (CIDP), chronic inducible urticaria, chronic spontaneous urticaria, Churg-Strauss syndrome, cicatrical pemphigoid, CREST syndrome, cold agglutinin disease, Crohn's disease, dermatomyositis, dilated cardiomyopathy, discoid lupus, epidermolysis bullosa acquisita, essential mixed cryoglobulinemia, factor VIII deficiency, fibromyalgia-fibromyositis, glomerulonephritis, Grave's disease, Guillain-Barre, Goodpasture's syndrome, graft-versus-host disease (GVHD), Hashimoto's thyroiditis, hemophilia A, idiopathic membranous neuropathy, idiopathic pulmonary fibrosis, idiopathic thrombocytopenia purpura (ITP), IgA neuropathy, IgM polyneuropathies, immune mediated thrombocytopenia, juvenile arthritis, Kawasaki's disease, lichen plantus, lichen sclerosus, systemic lupus erythematosis, lupus nephritis, Meniere's disease, mixed connective tissue disease, mucous membrane pemphigoid, multiple sclerosis, type 1 diabetes mellitus, Multifocal motor neuropathy (MMN), myasthenia gravis, paraneoplastic bullous pemphigoid, pemphigoid gestationis, pemphigus vulgaris, pemphigus foliaceus, pernicious anemia, polyarteritis nodosa, polychrondritis, polyglandular syndromes, polymyalgia rheumatica, polymyositis and dermatomyositis, primary agammaglobinulinemia, primary biliary cirrhosis, psoriasis, psoriatic arthritis, relapsing polychondritis, Reynauld's phenomenon, Reiter's syndrome, rheumatoid arthritis, sarcoidosis, scleroderma, Sjorgen's syndrome, solid organ transplant rejection, stiff-man syndrome, systemic lupus erythematosus, takayasu arteritis, toxic epidermal necrolysis (TEN), Stevens Johnson syndrome (SJS), temporal arteristis/giant cell arteritis, thrombotic thrombocytopenia purpura, ulcerative colitis, uveitis, dermatitis herpetiformis vasculitis, anti-neutrophil cytoplasmic antibody-associated vasculitides, vitiligo, and Wegener's granulomatosis.

In preferred embodiments, the autoimmune disease is chronic spontaneous urticaria or bullous pemphigoid. Thus, provided herein is an anti-IgE antibody or pharmaceutical composition of the invention for use in the treatment of chronic spontaneous urticaria or bullous pemphigoid.

In certain embodiments, the anti-IgE antibody or pharmaceutical composition may be administered to the subject simultaneously or sequentially with an additional therapeutic agent.

### BRIEF DESCRIPTION OF DRAWINGS

FIG. 1 shows the results of testing the pre-immune (PRE) and post-immune (POST) serum of immunized llamas for binding to human IgE.

FIG. 2 shows the binding of anti-IgE mAbs to human IgE, as measured by ELISA. Binding was measured at pH 5.5 and pH 7.4. (A) clone 3D6; (B) clone 16E4; (C) clone 3A1; (D) clone 3D1; (E) clone 13E4; (F) clone 18B9; (G) clone 20D5; (H) clone 18E2.

FIG. 3 shows the ability of anti-IgE mAbs to inhibit hIgE binding to hFcεRIα, as measured by ELISA. Binding was measured at pH 6 and pH 7.4. (A) clone 3D6; (B) clone 16E4; (C) clone 3A1; (D) clone 3D1; (E) clone 13E4; (F) clone 18B9; (G) clone 20D5; (H) clone 18E2.

FIG. 4 shows the ability of anti-IgE mAbs to inhibit hIgE binding to hFcεRIα, as determined by SPR analysis. Binding was measured at pH 6 and pH 7.4. (A) clone 3D6; (B) clone 16E4; (C) clone 3A1; (D) clone 3D1; (E) clone 13E4; (F) clone 18B9; (G) clone 20D5.

FIG. 5 shows the binding of anti-IgE mAbs to cynomolgus IgE, as measured by ELISA. Binding was measured at pH 5.5 and pH 7.4. (A) clone 3D6; (B) clone 16E4; (C) clone 3A1; (D) clone 3D1; (E) clone 13E4; (F) clone 18B9; (G) clone 20D5; (H) clone 18E2.

FIG. 6 shows the binding of anti-IgE ABDEG™ mAbs to human IgE, as measured by ELISA. Binding was measured at pH 5.5 and pH 7.4. (A) clone 18B9His; (B) clone 18E2His2; (C) clone 13E4.

FIG. 7 shows the ability of anti-IgE ABDEG™ mAbs to bind to FcRn with higher affinity as compared with the corresponding anti-IgE mAbs lacking the ABDEG™ technology. Efgartigimod (an isolated variant Fc molecule incorporating the ABDEG™ technology) was included for comparison. (A) Binding of clone 18B9His at pH 6.0; (B) Binding of clone 18B9His at pH 7.0; (C) Binding of clone 18E2His2 at pH 6.0; (D) Binding of clone 18E2His2 at pH 7.0; (E) Binding of clone 13E4 at pH 6.0; (F) Binding of clone 13E4 at pH 7.0.

FIG. 8 shows the ability of anti-IgE ABDEG™ mAbs to compete with native IgG3 for binding to FcRn, as measured by competition ELISA. (A) clone 18B9His; (B) clone 18E2His2; (C) clone 13E4.

FIG. 9 shows the ability of anti-IgE mAbs (both with and without ABDEG™) to inhibit IgE binding to hFcεRIα expressing mast cells. (A) clone 18B9His; (B) clone 18E2His2; (C) clone 13E4.

FIG. 10 shows the ability of anti-IgE mAbs (both with and without ABDEG™) to bind to hIgE pre-bound to hFcεRIα on mast cells, as measured by ELISA. (A) clone 13E4; (B) clone 18B9His; (C) clone 18E2His2.

FIG. 11 shows the ability of an anti-IgE ABDEG™ mAb to deplete both IgG (A) and IgE (B) levels in vivo. The controls used were: omalizumab (an anti-IgE antibody without ABDEG™ substitutions in the Fc domain) and HEL-hIgG1-ABDEG (an IgG1 antibody incorporating ABDEG™ substitutions but without binding specificity for IgE).

FIG. 12 shows a schematic of the method used to engineer pH-dependent variants of the anti-IgE antibody clone CL-2C (ligelizumab).

FIG. 13 shows the distribution of histidine residues at the various CDR positions of the Vκ (A) and VH (B) domains post-screening for CL-2C variant clones exhibiting pH-dependent binding to IgE.

FIG. 14 shows the ability of anti-IgE ABDEG™ mAbs to inhibit IgE binding to hFcεRIα expressing mast cells.

FIG. 15 shows the results of testing various anti-IgE antibodies in a mast cell activation assay. Bone marrow-derived mast cells were sensitized with IgE so as to load the FcεRIα receptor. The mast cells were subsequently incubated with various anti-IgE antibodies so as to test for the ability of these antibodies to cross-link the FcεRIα-bound IgE and trigger mast cell activation. (A) shows mast cell challenge with 20 μg/ml antibody; (B) shows mast cell challenge with 200 μg/ml antibody; (C) shows mast cell challenge with increasing concentrations of the clones 13E4-hIgG1-ABDEG™; 18B9-hIgG1-ABDEG™; and 18E2His2-MG-ABDEG™.

FIG. 16 shows the results of testing various anti-IgE antibodies for the induction of an anaphylactic reaction in vivo. Mice sensitized with recombinant human IgE were challenged with various anti-IgE antibodies and the temperature of the mice post-challenge was recorded at 15 minute intervals over a period of 2 hours. (A) and (B) show temperature changes over the time course of the experiment for antibodies administered at a dose of 15 mg/kg; (C) shows temperature changes over the time course of the experiment for antibodies administered at a dose of 50 mg/kg.

FIG. 17 shows the results of testing an ABDEG™ antibody in an in vivo model of Bullous Pemphigoid. Knock-in human NC16A mice were injected with either anti-hNC16A IgG or anti-hNC16A IgE in the presence or absence of an anti-IgE-ABDEG™ antibody. (A) shows the effect on skin disease score in mice injected with anti-hNC16A IgG and (B) shows the effect on the anti-hNC16A IgG levels in mice treated with or without a HEL-ABDEG™ antibody. (C) shows the effect on skin disease score in mice injected with anti-hNC16A IgE and (B) shows the effect on eosinophil peroxidase (EPO) activity in mice treated with or without an anti-IgE-ABDEG™ antibody. *p<0.001.

### DETAILED DESCRIPTION

### A. Definitions

Unless defined otherwise, all technical and scientific terms used herein have the same meaning as is commonly understood by one skilled in the art in the technical field of the invention.

“Antibody”—As used herein, the term “antibody” is intended to encompass full-length antibodies and variants thereof, including but not limited to modified antibodies, humanised antibodies, germlined antibodies (see definitions below). The term “antibody” is typically used herein to refer to immunoglobulin polypeptides having a combination of two heavy and two light chains wherein the polypeptide has significant specific immunoreactive activity to an antigen of interest (herein IgE). For antibodies of the IgG class, the antibodies comprise two identical light polypeptide chains of molecular weight approximately 23,000 Daltons, and two identical heavy chains of molecular weight 53,000-70,000. The four chains are joined by disulfide bonds in a “Y” configuration wherein the light chains bracket the heavy chains starting at the mouth of the “Y” and continuing through the variable region. The light chains of an antibody are classified as either kappa or lambda (κ,λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA, IgD or IgE, respectively. The immunoglobulin subclasses (isotypes) e.g., IgG1, IgG2, IgG3, IgG4, IgA1, etc. are well characterized and are known to confer functional specialization. The term “antibody” as used herein encompasses antibodies from any class or subclass of antibody.

“Variable region” or “variable domain”—The terms “variable region” and “variable domain” are used herein interchangeably and are intended to have equivalent meaning. The term “variable” refers to the fact that certain portions of the variable domains VH and VL differ extensively in sequence among antibodies and are used in the binding and specificity of each particular antibody for its target antigen. However, the variability is not evenly distributed throughout the variable domains of antibodies. It is concentrated in three segments called “hypervariable loops” in each of the VL domain and the VH domain which form part of the antigen binding site. The first, second and third hypervariable loops of the VLambda light chain domain are referred to herein as L1(λ), L2(λ) and L3(λ) and may be defined as comprising residues 24-33 (L1 (λ), consisting of 9, 10 or 11 amino acid residues), 49-53 (L2(λ), consisting of 3 residues) and 90-96 (L3(λ), consisting of 5 residues) in the VL domain (Morea et al., Methods 20:267-279 (2000)). The first, second and third hypervariable loops of the VKappa light chain domain are referred to herein as L1(λ), L2(λ) and L3(λ) and may be defined as comprising residues 25-33 (L1(λ), consisting of 6, 7, 8, 11, 12 or 13 residues), 49-53 (L2(λ), consisting of 3 residues) and 90-97 (L3(λ), consisting of 6 residues) in the VL domain (Morea et al., Methods 20:267-279 (2000)). The first, second and third hypervariable loops of the VH domain are referred to herein as H1, H2 and H3 and may be defined as comprising residues 25-33 (H1, consisting of 7, 8 or 9 residues), 52-56 (H2, consisting of 3 or 4 residues) and 91-105 (H3, highly variable in length) in the VH domain (Morea et al., Methods 20:267-279 (2000)).

Unless otherwise indicated, the terms L1, L2 and L3 respectively refer to the first, second and third hypervariable loops of a VL domain, and encompass hypervariable loops obtained from both Vkappa and Vlambda isotypes. The terms H1, H2 and H3 respectively refer to the first, second and third hypervariable loops of the VH domain, and encompass hypervariable loops obtained from any of the known heavy chain isotypes, including γ, ε, δ, α or μ.

The hypervariable loops L1, L2, L3, H1, H2 and H3 may each comprise part of a “complementarity determining region” or “CDR”, as defined below. The terms “hypervariable loop” and “complementarity determining region” are not strictly synonymous, since the hypervariable loops (HVs) are defined on the basis of structure, whereas complementarity determining regions (CDRs) are defined based on sequence variability (Kabat et al., Sequences of Proteins of Immunological Interest, 5th Ed. Public Health Service, National Institutes of Health, Bethesda, MD., 1983) and the limits of the HVs and the CDRs may be different in some VH and VL domains.

The CDRs of the VL and VH domains can typically be defined as comprising the following amino acids: residues 24-34 (LCDR1), 50-56 (LCDR2) and 89-97 (LCDR3) in the light chain variable domain, and residues 31-35 or 31-35b (HCDR1), 50-65 (HCDR2) and 95-102 (HCDR3) in the heavy chain variable domain; (Kabat et al., Sequences of Proteins of Immunological Interest, 5th Ed. Public Health Service, National Institutes of Health, Bethesda, MD. (1991)). Thus, the HVs may be comprised within the corresponding CDRs and references herein to the “hypervariable loops” of VH and VL domains should be interpreted as also encompassing the corresponding CDRs, and vice versa, unless otherwise indicated.

The more highly conserved portions of variable domains are called the framework region (FR), as defined below. The variable domains of native heavy and light chains each comprise four FRs (FR1, FR2, FR3 and FR4, respectively), largely adopting a β-sheet configuration, connected by the three hypervariable loops. The hypervariable loops in each chain are held together in close proximity by the FRs and, with the hypervariable loops from the other chain, contribute to the formation of the antigen-binding site of antibodies. Structural analysis of antibodies revealed the relationship between the sequence and the shape of the binding site formed by the complementarity determining regions (Chothia et al., J. Mol. Biol. 227: 799-817 (1992)); Tramontano et al., J. Mol. Biol, 215:175-182 (1990)). Despite their high sequence variability, five of the six loops adopt just a small repertoire of main-chain conformations, called “canonical structures”. These conformations are first of all determined by the length of the loops and secondly by the presence of key residues at certain positions in the loops and in the framework regions that determine the conformation through their packing, hydrogen bonding or the ability to assume unusual main-chain conformations.

“CDR”—As used herein, the term “CDR” or “complementarity determining region” means the non-contiguous antigen binding sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other. The amino acid residues which encompass the CDRs as defined by each of the above cited references are set forth for comparison. Preferably, the term “CDR” is a CDR as defined by Kabat based on sequence comparisons.

TABLE 1
CDR definitions

| | CDR Definitions | | |
|---|---|---|---|
| | Kabat¹ | Chothia² | MacCallum³ |
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

“Framework region”—The term “framework region” or “FR region” as used herein, includes the amino acid residues that are part of the variable region, but are not part of the CDRs (e.g., using the Kabat definition of CDRs). Therefore, a variable region framework is between about 100-120 amino acids in length but includes only those amino acids outside of the CDRs. For the specific example of a heavy chain variable domain and for the CDRs as defined by Kabat et al., framework region 1 corresponds to the domain of the variable region encompassing amino acids 1-30; framework region 2 corresponds to the domain of the variable region encompassing amino acids 36-49; framework region 3 corresponds to the domain of the variable region encompassing amino acids 66-94, and framework region 4 corresponds to the domain of the variable region from amino acids 103 to the end of the variable region. The framework regions for the light chain are similarly separated by each of the light chain variable region CDRs. Similarly, using the definition of CDRs by Chothia et al. or McCallum et al. the framework region boundaries are separated by the respective CDR termini as described above. In preferred embodiments the CDRs are as defined by Kabat.

In naturally occurring antibodies, the six CDRs present on each monomeric antibody are short, non-contiguous sequences of amino acids that are specifically positioned to form the antigen binding site as the antibody assumes its three dimensional configuration in an aqueous environment. The remainder of the heavy and light variable domains show less inter-molecular variability in amino acid sequence and are termed the framework regions. The framework regions largely adopt a β-sheet conformation and the CDRs form loops which connect, and in some cases form part of, the β-sheet structure. Thus, these framework regions act to form a scaffold that provides for positioning the six CDRs in correct orientation by inter-chain, non-covalent interactions. The antigen binding site formed by the positioned CDRs defines a surface complementary to the epitope on the immunoreactive antigen. This complementary surface promotes the non-covalent binding of the antibody to the immunoreactive antigen epitope. The position of CDRs can be readily identified by one of ordinary skill in the art.

“Constant region”—As used herein, the term “constant region” refers to the portion of the antibody molecule outside of the variable domains or variable regions. Immunoglobulin light chains have a single domain “constant region”, typically referred to as the “CL” or “CL1 domain”. This domain lies C terminal to the VL domain. Immunoglobulin heavy chains differ in their constant region depending on the class of immunoglobulin (γ, μ, α, δ, ε). Heavy chains γ, α and δ have a constant region consisting of three immunoglobulin domains (referred to as CH1, CH2 and CH3) with a flexible hinge region separating the CH1 and CH2 domains. Heavy chains μ and ε have a constant region consisting of four domains (CH1-CH4). The constant domains of the heavy chain are positioned C terminal to the VH domain.

The numbering of the amino acids in the heavy and light immunoglobulin chains run from the N-terminus at the forked ends of the Y configuration to the C-terminus at the bottom of each chain. Different numbering schemes are used to define the constant domains of the immunoglobulin heavy and light chains. In accordance with the EU numbering scheme, the heavy chain constant domains of an IgG molecule are identified as follows: CH1—amino acid residues 118-215; CH2—amino acid residues 231-340; CH3—amino acid residues 341-446. In accordance with the Kabat numbering scheme, the heavy chain constant domains of an IgG molecule are identified as follows: CH1—amino acid residues 114-223; CH2—amino acid residues 244-360; CH3—amino acid residues 361-477.

“Fc domain”—As used herein, the “Fc domain” defines the portion of the constant region of an immunoglobulin heavy chain including the CH2 and CH3 domains. It typically defines the portion of a single immunoglobulin heavy chain beginning in the hinge region just upstream of the papain cleavage site and ending at the C-terminus of the antibody. The Fc domain typically includes some residues from the hinge region. Accordingly, a complete Fc domain typically comprises at least a portion of a hinge (e.g., upper, middle, and/or lower hinge region) domain, a CH2 domain, and a CH3 domain.

The “hinge region” includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux K. H. et al. J. Immunol. 161:4083-90 1998). Antibodies of the invention comprising a “fully human” hinge region may contain one of the hinge region sequences shown in Table 2 below.

TABLE 2
Human hinge sequences

| IgG | Upper hinge | Middle hinge | Lower hinge |
|---|---|---|---|
| IgG1 | EPKSCDKTHT | CPPCP | APELLGGP |
| | (SEQ ID NO: 159) | (SEQ ID NO: 160) | (SEQ ID NO: 161) |
| IgG3 | ELKTPLGDTTHT | CPRCP(EPKSCDTPPPCPRCP)₃ | APELLGGP |
| | (SEQ ID NO: 162) | (SEQ ID NO: 163) | (SEQ ID NO: 164) |
| IgG4 | ESKYGPP | CPSCP | APEFLGGP |
| | (SEQ ID NO: 165) | (SEQ ID NO: 166) | (SEQ ID NO: 167) |
| IgG2 | ERK | CCVECPPPCP | APPVAGP |
| | (SEQ ID NO: 168) | (SEQ ID NO: 169) | (SEQ ID NO: 170) |

“Variant Fc domain”—As used herein, the term “variant Fc domain” refers to an Fc domain with one or more alterations relative to a wild-type Fc domain, for example the Fc domain of a naturally-occurring or “wild-type” human IgG. Alterations can include amino acid substitutions, additions and/or deletions, linkage of additional moieties, and/or alteration of the native glycans.

“Fc region”—As used herein, the term “Fc region” refers to the portion of a native immunoglobulin formed by the Fc domains of the two heavy chains. A native or wild-type Fc region is typically homodimeric.

“Variant Fc region”—As used herein the term “variant Fc region” refers to an Fc region wherein at least one of the Fc domains has one or more alterations relative to the wild-type domains of the wild-type Fc region, for example the Fc region of a naturally-occurring human IgG. In certain embodiments the term encompasses homodimeric Fc regions wherein each of the constituent Fc domains is the same. In certain embodiments the term encompasses heterodimeric Fc regions wherein each of the constituent Fc domains is different. For heterodimeric embodiments, one or both of the Fc domains may be variant Fc domains.

“FcRn binding fragment”—As used herein the term “FcRn binding fragment” refers to a portion of an Fc domain or Fc region that is sufficient to confer FcRn binding.

“Specificity” and “Multispecific antibodies”—The antibodies described herein bind to a particular target antigen, IgE. It is preferred that the antibodies “specifically bind” to their target antigen, wherein the term “specifically bind” refers to the ability of any antibody to preferentially immunoreact with a given target e.g. IgE. The antibodies of the present invention may be monospecific and contain one or more binding sites which specifically bind a particular target.

The antibodies may be incorporated into “multispecific antibody” formats, for example bispecific antibodies, wherein the multispecific antibody binds to two or more target antigens. In order to achieve multiple specificities, “multispecific antibodies” are typically engineered to include different combinations or pairings of heavy and light chain polypeptides with different VH-VL pairs. Multispecific, notably bispecific antibodies, may be engineered so as to adopt the overall conformation of a native antibody, for example a Y-shaped antibody having Fab arms of different specificities conjugated to an Fc region. Alternatively multispecific antibodies, for example bispecific antibodies, may be engineered so as to adopt a non-native conformation, for example wherein the variable domains or variable domain pairs having different specificities are positioned at opposite ends of the Fc region.

“Modified antibody”—As used herein, the term “modified antibody” includes synthetic forms of antibodies which are altered such that they are not naturally occurring. Examples include but are not limited to antibodies that comprise at least two heavy chain portions but not two complete heavy chains (such as, domain deleted antibodies or minibodies); multispecific forms of antibodies (e.g., bispecific, trispecific, etc.) altered to bind to two or more different antigens or to different epitopes on a single antigen); heavy chain molecules joined to scFv molecules and the like. scFv molecules are known in the art and are described, e.g., in U.S. Pat. No. 5,892,019. In addition, the term “modified antibody” includes multivalent forms of antibodies (e.g., trivalent, tetravalent, etc., antibodies that bind to three or more copies of the same antigen).

The term “modified antibody” may also be used herein to refer to amino acid sequence variants of the antibodies of the invention as structurally defined herein. It will be understood by one of ordinary skill in the art that an antibody may be modified to produce a variant antibody which varies in amino acid sequence in comparison to the antibody from which it was derived. For example, nucleotide or amino acid substitutions leading to conservative substitutions or changes at “non-essential” amino acid residues may be made (e.g., in CDR and/or framework residues). Amino acid substitutions can include replacement of one or more amino acids with a naturally occurring or non-natural amino acid.

Modified antibodies in accordance with the present invention may comprise any suitable antigen-binding fragment as defined herein linked to a variant Fc domain or FcRn binding fragment thereof as defined in accordance with the invention.

“Antigen binding fragment”—The term “antigen binding fragment” as used herein refers to fragments that are parts or portions of a full-length antibody or antibody chain comprising fewer amino acid residues than an intact or complete antibody whilst retaining antigen binding activity.

An antigen-binding fragment of an antibody includes peptide fragments that exhibit specific immuno-reactive activity to the same antigen as the antibody (e.g. IgE). The term “antigen binding fragment” as used herein is intended to encompass antibody fragments selected from: an antibody light chain variable domain (VL); an antibody heavy chain variable domain (VH); a VH-VL domain pairing; a single chain antibody (scFv); a F(ab′)2 fragment; a Fab fragment; an Fd fragment; an Fv fragment; a one-armed (monovalent) antibody; diabodies, triabodies, tetrabodies or any antigen-binding molecule formed by combination, assembly or conjugation of such antigen binding fragments. The term “antigen binding fragment” as used herein may also encompass antibody fragments selected from the group consisting of: unibodies; domain antibodies; and nanobodies. Fragments can be obtained, for example, via chemical or enzymatic treatment of an intact or complete antibody or antibody chain or by recombinant means.

“Humanising substitutions”—As used herein, the term “humanising substitutions” refers to amino acid substitutions in which the amino acid residue present at a particular position in the VH or VL domain of an antibody is replaced with an amino acid residue which occurs at an equivalent position in a reference human VH or VL domain. The reference human VH or VL domain may be a VH or VL domain encoded by the human germline. Humanising substitutions may be made in the framework regions and/or the CDRs of the antibodies, defined herein.

“Humanised variants”—As used herein the term “humanised variant” or “humanised antibody” refers to a variant antibody which contains one or more “humanising substitutions” compared to a reference antibody, wherein a portion of the reference antibody (e.g. the VH domain and/or the VL domain or parts thereof containing at least one CDR) has an amino acid derived from a non-human species, and the “humanising substitutions” occur within the amino acid sequence derived from a non-human species.

“Germlined variants”—The term “germlined variant” or “germlined antibody” is used herein to refer specifically to “humanised variants” in which the “humanising substitutions” result in replacement of one or more amino acid residues present at (a) particular position(s) in the VH or VL domain of an antibody with an amino acid residue which occurs at an equivalent position in a reference human VH or VL domain encoded by the human germline. It is typical that for any given “germlined variant”, the replacement amino acid residues substituted into the germlined variant are taken exclusively, or predominantly, from a single human germline-encoded VH or VL domain. The terms “humanised variant” and “germlined variant” are often used interchangeably. Introduction of one or more “humanising substitutions” into a camelid-derived (e.g. llama derived) VH or VL domain results in production of a “humanised variant” of the camelid (llama)-derived VH or VL domain. If the amino acid residues substituted in are derived predominantly or exclusively from a single human germline-encoded VH or VL domain sequence, then the result may be a “human germlined variant” of the camelid (llama)-derived VH or VL domain.

“Affinity variants”—As used herein, the term “affinity variant” refers to a variant antibody which exhibits one or more changes in amino acid sequence compared to a reference antibody, wherein the affinity variant exhibits an altered affinity for the target antigen in comparison to the reference antibody. For example, affinity variants will exhibit a changed affinity for a target, for example IgE, as compared to the reference IgE antibody. Preferably the affinity variant will exhibit improved affinity for the target antigen, as compared to the reference antibody. Affinity variants typically exhibit one or more changes in amino acid sequence in the CDRs, as compared to the reference antibody. Such substitutions may result in replacement of the original amino acid present at a given position in the CDRs with a different amino acid residue, which may be a naturally occurring amino acid residue or a non-naturally occurring amino acid residue. The amino acid substitutions may be conservative or non-conservative.

“Engineered”—As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques). Preferably, the antibodies of the invention are engineered, including for example, humanized antibodies which have been engineered to improve one or more properties, such as antigen binding, stability/half-life or effector function.

“FcRn”—As used herein, the term “FcRn” refers to a neonatal Fc receptor. Exemplary FcRn molecules include human FcRn encoded by the FCGRT gene as set forth in RefSeq NM_004107.

“CD16”—As used herein, the term “CD16” refers to FcγRIII Fc receptors that are required for Antibody-Dependent Cell-mediated Cytotoxicity (ADCC). Exemplary CD16 molecules include human CD16a as set forth in RefSeq NM_000569.

“N-linked glycan”—As used herein the term “N-linked glycan” refers to the N-linked glycan attached to the nitrogen (N) in the side chain of asparagine in the sequence (i.e., Asn-X-Ser or Asn-X-Thr sequence, where X is any amino acid except proline) present in the CH2 domain of an Fc region. Such N-glycans are fully described in, for example, Drickamer K and Taylor M E (2006) Introduction to Glycobiology, 2nd ed., incorporated herein by reference in its entirety.

“Afucosylated”—As used herein the term “afucosylated” refers to an N-linked glycan which lacks a core fucose molecule as described in U.S. Pat. No. 8,067,232, incorporated herein by reference in its entirety.

“Bisecting GlcNAc”—As used herein the term “bisecting GlcNAc” refers to an N-linked glycan having an N-acetylglucosamine (GlcNAc) molecule linked to a core mannose molecule, as described in U.S. Pat. No. 8,021,856, incorporated herein by reference in its entirety.

“IgE”—As used herein, the term “IgE” refers to “immunoglobulin E” molecules or “class E immunoglobulins”. IgE is the least abundant immunoglobulin isotype in human serum. IgE immunoglobulins adopt the tetrameric structure common to other classes or isotypes of immunoglobulin. However, IgE is characterised by its s heavy chains, which comprise four constant regions: Cε1, Cε2, Cε3 and Cε4 (also referred to herein as CH1, CH2, CH3 and CH4). As explained elsewhere herein, IgE plays an important role in allergy and hypersensitivity by binding to the high-affinity Fc receptors on mast cells and basophils. This high-affinity receptor, FcεRI, has a multisubunit structure including one IgE-binding a subunit, one β subunit and a dimer of disulphide-linked γ subunits. A low-affinity IgE receptor, FcαRII (also known as CD23), is constitutively expressed on B cells and can be expressed on macrophages, eosinophils, platelets and some T cells in response to IL-4.

Omalizumab—Omalizumab is a recombinant humanized monoclonal antibody that binds to IgE. It contains 5% murine sequence and 95% human sequence. It is marketed by Novartis as Xolair®, and is approved for the treatment of allergic asthma and Chronic Spontaneous Urticaria (CSU). The CDR, VH and VL sequences of omalizumab are shown in table 3 below.

TABLE 3
CDR, VH and VL sequences for omalizumab

| | Sequence | SEQ ID NO. |
|---|---|---|
| VH CDR1 | SGYSWN | 143 |
| VH CDR2 | SITYDGSTNYNPSVKG | 144 |
| VH CDR3 | GSHYFGHWHFAV | 145 |
| VH | EVQLVESGGGLVQPGGSLRLSCAVSGYSITSGYSWNWIRQAP | 146 |
| | GKGLEWVASITYDGSTNYNPSVKGRITISRDDSKNTFYLQMNSL | |
| | RAEDTAVYYCARGSHYFGHWHFAVWGQGTLVTVSS | |
| VL CDR1 | RASQSVDYDGDSYMN | 147 |
| VL CDR2 | AASYLES | 148 |
| VL CDR3 | QQSHEDPYT | 149 |
| VL | DIQLTQSPSSLSASVGDRVTITCRASQSVDYDGDSYMNWYQQK | 150 |
| | PGKAPKLLIYAASYLESGVPSRFSGSGSGTDFTLTISSLQPEDFA | |
| | TYYCQQSHEDPYTFGQGTKVEIK | |

Omalizumab binds to the receptor-binding portion of IgE i.e. a region within the CH3 or Cε3 domain. Since the epitope that is recognized by omalizumab encompasses binding regions for both the high-affinity and low-affinity IgE receptors, omalizumab eliminates the ability of IgE to bind to both types of receptor. Importantly, omalizumab is not able to cross-link IgE molecules that are already bound on the cell surface i.e. it is non-anaphylactogenic. The binding of FcεRI to one CH3 domain of one IgE heavy chain inhibits or prevents the binding of omalizumab to the CH3 region of the other IgE heavy chain. Thus, omalizumab can only bind to IgE that is in circulation. In the circulation, each molecule of IgE can be simultaneously bound by two molecules of omalizumab.

Ligelizumab—Ligelizumab is a second humanized monoclonal antibody that binds to IgE. It binds to the same region of IgE as omalizumab but binds to IgE with higher affinity. The CDR, VH and VL sequences of ligelizumab are shown in table 4 below.

TABLE 4
CDR, VH and VL sequences for ligelizumab

| | Sequence | SEQ ID NO. |
|---|---|---|
| VH CDR1 | WYWLE | 151 |
| VH CDR2 | EIDPGTFTTNYNEKFKA | 152 |
| VH CDR3 | FSHFSGSNYDYFDY | 153 |
| VH | QVQLVQSGAEVMKPGSSVKVSCKASGYTFSWYWLEWVRQAP | 154 |
| | GHGLEWMGEIDPGTFTTNYNEKFKARVTFTADTSTSTAYMELS | |
| | SLRSEDTAVYYCARFSHFSGSNYDYFDYWGQGTLVTVSS | |
| VL CDR1 | RASQSIGTNIH | 155 |
| VL CDR2 | YASESIS | 156 |
| VL CDR3 | QQSWSWPTT | 157 |
| VL | EIVMTQSPATLSVSPGERATLSCRASQSIGTNIHWYQQKPGQAP | 158 |
| | RLLIYYASESISGIPARFSGSGSGTEFTLTISSLQSEDFAVYYCQQ | |
| | SWSWPTTFGGGTKVEIK | |

“Antibody-mediated disorder”—As used herein, the term “antibody-mediated disorder” refers to any disease or disorder caused or exacerbated by the presence of an antibody in a subject.

“Treat, treating and treatment”—As used herein, the terms “treat,” “treating,” and “treatment” refer to therapeutic or preventative measures described herein. The methods of “treatment” employ administration to a subject, for example, a subject having an antibody-mediated disease or disorder (e.g. autoimmune disease) or predisposed to having such a disease or disorder, an antibody in accordance with the present invention, in order to prevent, cure, delay, reduce the severity of, or ameliorate one or more symptoms of the disease or disorder or recurring disease or disorder, or in order to prolong the survival of a subject beyond that expected in the absence of such treatment.

“Subject”—As used herein, the term “subject” refers to any human or non-human animal. In certain embodiments, the term “subject” refers to any human or non-human mammal. In preferred embodiments, the subject is a human. In certain embodiments the subject is an adult human. As used herein, an “adult human” is a human who is at least 18 years of age.

### B. Anti-IgE Antibodies Having Variant Fc Domains

### (i) Variant Fc Domains and FcRn Binding Fragments Thereof

In a first aspect, the present invention provides antibodies that bind to IgE (i.e. anti-IgE antibodies) wherein the antibodies comprise at least one variant Fc domain or FcRn binding fragment thereof. This variant Fc domain or FcRn binding fragment thereof is characterised by the ability to bind to the neonatal Fc receptor, FcRn, with increased affinity relative to a wild-type Fc domain. Put another way, the binding affinity between the variant Fc domain or FcRn binding fragment of the anti-IgE antibodies described herein and FcRn is higher as compared with the binding affinity between a wild-type Fc domain and FcRn.

The FcRn receptor plays an important role in regulating IgG concentrations in the plasma by means of the salvage receptor pathway. The model for FcRn function is as follows. IgGs in the circulation are taken up into cells, most likely by fluid-phase pinocytosis, as the near-neutral pH of the extracellular milieu is generally not permissive for FcRn-IgG interactions. IgGs that bind to FcRn in early, acidic endosomes following uptake are recycled (or transcytosed) and released at the cell surface by exocytosis. In contrast, IgGs that do not bind FcRn, enter the lysosomal pathway and are degraded.

By virtue of binding with higher affinity to FcRn, the anti-IgE antibodies of the invention interfere with the recycling of endogenous IgG molecules and thus can reduce the levels of endogenous IgG antibodies, for example IgG autoantibodies. It follows, that the anti-IgE antibodies of the invention target both endogenous IgE (by virtue of antigen binding via the variable region) and endogenous IgG (by competing for binding to FcRn via the variant Fc domain).

The variant Fc domains or FcRn binding fragments thereof bind to FcRn with increased affinity relative to a wild-type Fc domain. In certain embodiments, the wild-type Fc domain against which the binding affinity of the variant Fc domain is compared may be the wild-type Fc domain from which the variant Fc domain derives. As described above, a variant Fc domain in the context of the present invention refers to an Fc domain with one or more alterations relative to a wild-type Fc domain, for example the Fc domain of a naturally-occurring or “wild-type” human IgG. Alterations can include amino acid substitutions, additions and/or deletions, linkage of additional moieties, and/or alteration of the native glycans. If the naturally-occurring or wild-type Fc domain from which the variant Fc domain derives is a human IgG1 Fc domain, the variant Fc domain may bind to FcRn with higher affinity than the wild-type human IgG1 Fc domain.

The increased affinity for FcRn exhibited by the variant Fc domain or FcRn binding fragment may be relative to a wild-type Fc domain that is not necessarily the Fc domain from which the variant Fc domain or FcRn binding fragment derives. For example, the variant Fc domain or FcRn binding fragment thereof may bind to FcRn with increased affinity relative to a wild-type human IgG Fc domain. The wild-type human IgG may be an IgG1, IgG2, IgG3 or IgG4. In preferred embodiments, the variant Fc domain or FcRn binding fragment thereof of the anti-IgE antibodies described herein binds to FcRn with increased affinity relative to a wild-type human IgG1 Fc domain or a wild-type human IgG3 Fc domain. In a preferred embodiment, the variant Fc domain or FcRn binding fragment thereof of the anti-IgE antibodies described herein binds to FcRn with increased affinity relative to a wild-type human IgG1 Fc domain.

Since the anti-IgE antibodies of the present invention are intended for use in the treatment of human disease, particularly the depletion of IgG autoantibodies from patients having autoimmune diseases, the variant Fc region or FcRn binding fragment thereof will typically bind with higher affinity to human FcRn. In other words, the variant Fc region or FcRn binding fragment of the anti-IgE antibodies described herein will compete with native or endogenous patient IgG antibodies for binding to human FcRn.

The interaction between IgG Fc domains and FcRn is pH-dependent. The binding affinity is typically stronger at acidic pH (i.e. at the pH found in the early endosomal compartment) and weaker at neutral pH (i.e. plasma pH). The variant Fc domains or FcRn binding fragments described herein may bind to FcRn with increased affinity at acidic pH, for example pH 6.0. Alternatively or in addition, the variant Fc domains or FcRn binding fragments described herein may bind to FcRn with increased affinity at neutral pH, for example pH 7.4. In preferred embodiments, the variant Fc domains or FcRn binding fragments of the anti-IgE antibodies described herein bind to FcRn with increased affinity at both pH 6.0 and pH 7.4. In certain embodiments, the variant Fc domains and/or FcRn binding fragments bind to FcRn with reduced pH-dependence as compared with a wild-type Fc domain, particularly a wild-type human IgG1 Fc domain. For embodiments where the variant Fc domain or FcRn binding fragment binds to FcRn with reduced pH-dependence, it is still preferred that the binding affinity is increased at pH 6.0 and pH 7.4.

As explained herein, the binding affinity between the variant Fc domains or FcRn binding fragments described herein and FcRn is increased such that the antibodies of the present invention compete with endogenous IgGs, particularly IgG autoantibodies, for binding to FcRn. As reported in Vaccaro et al. (Engineering the Fc region of immunoglobulin G to modulate in vivo antibody levels. Nature Biotechnology (2005) 23(10): 1283-1288), Ulrichts et al. (Neonatal Fc receptor antagonist efgartigimod safely and sustainably reduces IgGs in humans. J. Clinical Investigation. (2018) 128(10): 4372-4386), and also reported herein, a variant Fc region comprising variant Fc domains having ABDEG™ mutations (M252Y/S254T/T256E/H433K/N434F) can bind to human FcRn with increased affinity and thereby reduce endogenous IgG levels. Vaccaro et al. (incorporated herein by reference) reports a binding affinity for human FcRn at pH 6.0 for the variant ABDEG™ Fc region of K_{D} 15.5 nM as compared with a binding affinity of K_{D} 370 nM for wild-type human IgG1 (as measured by surface plasmon resonance analysis). Thus, in certain embodiments, the variant Fc domain or FcRn binding fragments described herein bind to human FcRn at pH 6.0 with an affinity that is increased by at least 20× as compared with a wild-type human IgG1 Fc domain. In certain embodiments, the variant Fc domain or FcRn binding fragments described herein bind to human FcRn at pH 6.0 with an affinity that is increased by at least 25×, preferably at least 30×, as compared with a wild-type human IgG1 Fc domain. The binding affinity of the variant Fc domain or FcRn binding fragment may be compared with the binding affinity of the wild-type human IgG1 Fc domain when the affinity of the Fc domains (or fragment) is tested in the context of a full-length IgG molecule.

As reported in Ulrichts et al. the FcRn antagonist, Efgartigimod, has equilibrium dissociation constants (K_{D}) for human FcRn of 14.2 nM and 320 nM at pH 6.0 and pH 7.4, respectively.

Thus, in certain embodiments, the variant Fc domain or FcRn binding fragments described herein bind to human FcRn at pH 6.0 with a binding affinity stronger than K_{D} 15 nM. Alternatively or in addition, the variant Fc domain or FcRn binding fragments described herein may bind to human FcRn at pH 7.4 with a binding affinity stronger than K_{D} 320 nM. The binding affinity of the variant Fc domain or FcRn binding fragment thereof may be determined when the variant Fc domain or FcRn binding fragment thereof is tested in the context of a variant Fc region (i.e. including two Fc domains).

The variant Fc domains or FcRn binding fragments comprise one or more alterations relative to a wild-type Fc domain. In certain embodiments, the variant Fc domains or FcRn binding fragments comprise at least one amino acid substitution relative to a wild-type Fc domain. The variant Fc domains or FcRn binding fragments may comprise, in certain embodiments, at least two, at least three, at least four or at least five amino acid substitutions relative to a wild-type Fc domain.

The number of alterations in the variant Fc domain or FcRn binding fragment thereof may be limited relative to the corresponding wild-type Fc domain or FcRn binding fragment. For example, the total number of amino acid substitutions in the variant Fc domain or FcRn binding fragment may be limited relative to the corresponding wild-type Fc domain or FcRn binding fragment. In certain embodiments, the variant Fc domain or FcRn binding fragment thereof consists of no more than 5, no more than 6, no more than 7, no more than 8, no more than 9, no more than 10, no more than 11, no more than 12, no more than 15, no more than 20 alterations as compared with the corresponding wild-type Fc domain. The alterations may be selected from amino acid substitutions, additions and/or deletions, linkage of additional moieties, and/or alteration of the native glycans. In certain embodiments, the variant Fc domain or FcRn binding fragment thereof consists of no more than 5, no more than 6, no more than 7, no more than 8, no more than 9, no more than 10, no more than 11, no more than 12, no more than 15, no more than 20 amino acid substitutions as compared with the corresponding wild-type Fc domain.

In certain embodiments, the variant Fc domain or FcRn binding fragment thereof comprises or consists of at least one amino acid substitution but no more than 20 amino acid substitutions in total. In certain embodiments, the variant Fc domain or FcRn binding fragment thereof comprises or consists of at least two amino acid substitutions but no more than 20 amino acid substitutions in total. In certain embodiments, the variant Fc domain or FcRn binding fragment thereof comprises or consists of at least one amino acid substitution but no more than 10 amino acid substitutions in total. In certain embodiments, the variant Fc domain or FcRn binding fragment thereof comprises or consists of at least two amino acid substitutions but no more than 10 amino acid substitutions in total. In certain embodiments, the variant Fc domain or FcRn binding fragment thereof comprises or consists of at least one amino acid substitution but no more than 5 amino acid substitutions in total. In certain embodiments, the variant Fc domain or FcRn binding fragment thereof comprises or consists of at least two amino acid substitutions but no more than 5 amino acid substitutions in total.

The wild-type Fc domain from which the variant Fc domains of the anti-IgE antibodies described herein derive may be an IgG Fc domain. In such embodiments, the variant Fc domain is a variant IgG Fc domain. In preferred embodiments, the variant Fc domain is a variant IgG1 Fc domain i.e. the variant Fc domain possesses one or more alterations relative to a wild-type IgG1 domain.

Since the anti-IgE antibodies of the present invention may be for use in human patients, the variant Fc domains or FcRn binding fragments thereof will preferably be variant forms of human Fc domains i.e. the variant Fc domain or FcRn binding fragment thereof will be a variant human Fc domain or FcRn binding fragment thereof. Since the purpose of the variant Fc domain is to compete with native IgG antibodies for binding to FcRn, it is preferred that the variant Fc domain is a human variant IgG domain, for example a human variant IgG domain selected from IgG1, IgG2, IgG3 or IgG4. In particularly preferred embodiments, the variant Fc domain is a variant IgG1 Fc domain or FcRn binding fragment thereof.

The variant Fc domains or FcRn binding fragments of the anti-IgE antibodies of the present invention may comprise any non-native amino acid residues, provided that the variant Fc domain or FcRn binding fragment exhibits the requisite increased binding affinity for FcRn, preferably human FcRn. As used herein, the term “non-native amino acid” means an amino acid that does not occur naturally at the position at which it is located in the variant Fc domain or FcRn binding fragment thereof.

Antibodies having variant Fc domains and exhibiting increased binding affinity for FcRn have been reported in the literature. These variant Fc domains have been reported as having various non-native amino acids at specific positions within the Fc domain. The variant Fc domains and FcRn binding fragments of the anti-IgE antibodies described herein may comprise any of the non-native amino acids and/or amino acid substitutions described in the literature as capable of increasing Fc domain binding affinity for FcRn. The variant Fc domains and FcRn binding fragments of the anti-IgE antibodies described herein may also comprise any combinations of non-native amino acids and/or amino acid substitutions described in the literature as capable of increasing Fc domain binding affinity for FcRn. Non-limiting examples of amino acid substitutions that may be included in the variant Fc domains or FcRn binding fragments described herein are reported in Yeung et al. (Engineering Human IgG1 Affinity to Human Neonatal Fc Receptor: Impact of Affinity Improvement on Pharmacokinetics in Primates. J. Immunol. (2009) 182: 7663-7671), and also International patent application no. WO2011/122011, the entire contents of which are incorporated herein by reference.

In certain embodiments, the variant Fc domains or FcRn binding fragments described herein comprise at least one amino acid selected from the following: 237M; 238A; 239K; 248I; 250A; 250F; 2501; 250M; 250Q; 250S; 250V; 250W; 250Y; 252F; 252W; 252Y; 254T; 255E; 256D; 256E; 256Q; 257A; 257G; 2571; 257L; 257M; 257N; 257S; 257T; 257V; 258H; 265A; 270F; 286A; 286E; 289H; 297A; 298G; 303A; 305A; 307A; 307D; 307F; 307G; 307H; 307I; 307K; 307L; 307M; 307N; 307P; 307Q; 307R; 307S; 307V; 307W; 307Y; 308A; 308F; 3081; 308L; 308M; 308P; 308Q; 308T; 309A; 309D; 309E; 309P; 309R; 311A; 311H; 311I; 312A; 312H; 314K; 314R; 315A; 315H; 317A; 325G; 332V; 334L; 360H; 376A; 378V; 380A; 382A; 384A; 385D; 385H; 386P; 387E; 389A; 389S; 424A; 428A; 428D; 428F; 428G; 428H; 4281; 428K; 428L; 428N; 428P; 428Q; 428S; 428T; 428V; 428W; 428Y; 433K; 434A; 434F; 434H; 434S; 434W; 434Y; 436H; 4361 and 436F, wherein the positions are defined in accordance with EU numbering. EU numbering refers to the convention for the Fc region described in Edelman, G. M. et al., Proc. Natl. Acad. Sci. USA, 63: 78-85 (1969); and Kabat et al., in “Sequences of Proteins of Immunological Interest”, U.S. Dept. Health and Human Services, 5th edition, 1991. The variant Fc domains or FcRn binding fragments described herein may comprise 2, 3, 4 or 5 amino acids selected from the following: 237M; 238A; 239K; 248I; 250A; 250F; 2501; 250M; 250Q; 250S; 250V; 250W; 250Y; 252F; 252W; 252Y; 254T; 255E; 256D; 256E; 256Q; 257A; 257G; 2571; 257L; 257M; 257N; 257S; 257T; 257V; 258H; 265A; 270F; 286A; 286E; 289H; 297A; 298G; 303A; 305A; 307A; 307D; 307F; 307G; 307H; 307I; 307K; 307L; 307M; 307N; 307P; 307Q; 307R; 307S; 307V; 307W; 307Y; 308A; 308F; 3081; 308L; 308M; 308P; 308Q; 308T; 309A; 309D; 309E; 309P; 309R; 311A; 311H; 311I; 312A; 312H; 314K; 314R; 315A; 315H; 317A; 325G; 332V; 334L; 360H; 376A; 378V; 380A; 382A; 384A; 385D; 385H; 386P; 387E; 389A; 389S; 424A; 428A; 428D; 428F; 428G; 428H; 4281; 428K; 428L; 428N; 428P; 428Q; 428S; 428T; 428V; 428W; 428Y; 433K; 434A; 434F; 434H; 434S; 434W; 434Y; 436H; 4361 and 436F, wherein the positions are defined in accordance with EU numbering and wherein any combinations are contemplated.

In certain embodiments, the variant Fc domains or FcRn binding fragments described herein comprise a combination of amino acids selected from the following:
(i) Y, T, E, K, F and Y at EU positions 252, 254, 256, 433, 434 and 436, respectively;
(ii) Q and L at EU positions 250 and 428, respectively;
(iii) P and A at EU positions 308 and 434, respectively;
(iv) P and Y at EU positions 308 and 434, respectively; or
(v) Y, E and Y at EU positions 252, 286 and 434, respectively.

In certain embodiments, the variant Fc domains or FcRn binding fragments described herein comprise at least one amino acid substitution selected from: G237M; P238A; S239K; K248I; T250A; T250F; T2501; T250M; T250Q; T250S; T250V; T250W; T250Y; M252F; M252W; M252Y; S254T; R255E; T256D; T256E; T256Q; P257A; P257G; P2571; P257L; P257M; P257N; P257S; P257T; P257V; E258H; D265A; D270F; N286A; N286E; T289H; N297A; S298G; V303A; V305A; T307A; T307D; T307F; T307G; T307H; T307I; T307K; T307L; T307M; T307N; T307P; T307Q; T307R; T307S; T307V; T307W; T307Y; V308A; V308F; V3081; V308L; V308M; V308P; V308Q; V308T; V309A; V309D; V309E; V309P; V309R; Q311A; Q311H; Q311I; D312A; D312H; L314K; L314R; N315A; N315H; K317A; N325G; I332V; K334L; K360H; D376A; A378V; E380A; E382A; N384A; G385D; G385H; Q386P; P387E; N389A; N389S; S424A; M428A; M428D; M428F; M428G; M428H; M4281; M428K; M428L; M428N; M428P; M428Q; M428S; M428T; M428V; M428W; M428Y; H433K; N434A; N434F; N434H; N434S; N434W; N434Y; Y436H; Y4361 and Y436F, wherein the positions are defined in accordance with EU numbering. The variant Fc domains or FcRn binding fragments described herein may comprise 2, 3, 4 or 5 amino acid substitutions selected from the following: G237M; P238A; S239K; K248I; T250A; T250F; T2501; T250M; T250Q; T250S; T250V; T250W; T250Y; M252F; M252W; M252Y; S254T; R255E; T256D; T256E; T256Q; P257A; P257G; P2571; P257L; P257M; P257N; P257S; P257T; P257V; E258H; D265A; D270F; N286A; N286E; T289H; N297A; S298G; V303A; V305A; T307A; T307D; T307F; T307G; T307H; T307I; T307K; T307L; T307M; T307N; T307P; T307Q; T307R; T307S; T307V; T307W; T307Y; V308A; V308F; V3081; V308L; V308M; V308P; V308Q; V308T; V309A; V309D; V309E; V309P; V309R; Q311A; Q311H; Q311I; D312A; D312H; L314K; L314R; N315A; N315H; K317A; N325G; I332V; K334L; K360H; D376A; A378V; E380A; E382A; N384A; G385D; G385H; Q386P; P387E; N389A; N389S; S424A; M428A; M428D; M428F; M428G; M428H; M4281; M428K; M428L; M428N; M428P; M428Q; M428S; M428T; M428V; M428W; M428Y; H433K; N434A; N434F; N434H; N434S; N434W; N434Y; Y436H; Y4361 and Y436F, wherein the positions are defined in accordance with EU numbering, and wherein any combinations of substitutions are contemplated.

In certain embodiments, the variant Fc domains or FcRn binding fragments described herein comprise a combination of amino acid substitutions selected from the following:
(i) M252Y, S254T, T256E, H433K and N434F;
(ii) T250Q and M428L;
(iii) V308P and N434A;
(iv) V308P and N434Y; or
(v) M252Y, N286E and N434Y.

In certain embodiments, the variant Fc domains or FcRn binding fragments do not comprise the combination of amino acids Y, P and Y at EU positions 252, 308 and 434, respectively. In certain embodiments, the variant Fc domains or FcRn binding fragments do not comprise the combination of amino acid substitutions: M252Y, V308P and N434Y.

In certain embodiments, the anti-IgE antibodies of the invention comprise a variant Fc region consisting of two Fc domains or FcRn binding fragments thereof, wherein at least one of the Fc domains or FcRn binding fragments is a variant Fc domain or FcRn binding fragment as described herein. In certain embodiments, the two variant Fc domains of the variant Fc region are different and form a heterodimer. For heterodimeric embodiments, one or both of the Fc domains or FcRn binding fragments thereof may be a variant Fc domain or FcRn binding fragment thereof. In certain embodiments, the two variant Fc domains of the variant Fc region are identical and form a homodimer.

### (ii) Variant Fc Domains and FcRn Binding Fragments Thereof Incorporating ABDEG™

In preferred embodiments, the present invention provides antibodies that bind to IgE (i.e. anti-IgE antibodies) wherein the antibodies comprise at least one variant Fc domain incorporating ABDEG™ technology. As reported in Vaccaro et al. (Nat. Biotechnology (2005) 23(10):1283-8), ABDEG™ antibodies (meaning “antibodies that enhance IgG degradation”) comprise an engineered or variant Fc region. This engineered or variant Fc region can bind to the neonatal Fc receptor, FcRn, with higher affinity and reduced pH dependence as compared with the Fc region of wild-type antibodies.

As explained above, the FcRn receptor plays an important role in regulating IgG concentrations in the plasma by means of the salvage receptor pathway. By virtue of binding with higher affinity to FcRn, ABDEG™ antibodies interfere with the recycling of endogenous immunoglobulins and thus can reduce the levels of endogenous immunoglobulins, for example autoantibodies. ABDEG™ antibodies and FcRn antagonists incorporating ABDEG™ technology have been described for the treatment of antibody-mediated diseases such as autoimmune diseases (see WO2006/130834 and WO2015/100299, incorporated herein by reference).

The Fc domain amino acid “signature” of ABDEG™ antibodies is well-characterised. Therefore, in preferred embodiments, the present invention provides an antibody that binds to IgE, wherein the antibody comprises a variant Fc domain or a FcRn binding fragment thereof, said variant Fc domain or FcRn binding fragment thereof comprising the amino acids Y, T, E, K, F and Y at EU positions 252, 254, 256, 433, 434 and 436, respectively. This variant Fc domain is referred to herein as a variant ABDEG™ Fc domain.

As described above, the variant Fc domain of ABDEG™ antibodies is engineered so as to increase the binding affinity for the Fc receptor FcRn, particularly human FcRn. The variant ABDEG™ Fc domain or FcRn binding fragment thereof binds to FcRn with increased affinity relative to a wild-type Fc domain. In such embodiments, the wild-type Fc domain may be the wild-type Fc domain from which the variant Fc domain derives. For example, if the variant ABDEG™ Fc domain is derived from a human IgG1 Fc domain, the variant Fc domain may bind to FcRn with higher affinity than the human IgG1 Fc domain.

In certain embodiments, the variant ABDEG™ Fc domain or FcRn binding fragment thereof binds to FcRn, preferably human FcRn, with increased affinity relative to a wild-type IgG Fc domain, preferably a wild-type human IgG Fc domain. In a preferred embodiment, the variant ABDEG™ Fc domain or FcRn binding fragment thereof binds to FcRn, preferably human FcRn, with increased affinity relative to a wild-type human IgG1 Fc domain or a wild-type human IgG3 Fc domain. For anti-IgE antibodies of the invention that are intended for use in depleting human IgG autoantibodies, it is preferred that the variant ABDEG™ Fc domain or FcRn binding fragment thereof (irrespective of its origin) binds to human FcRn with increased affinity relative to the wild-type human IgG1 Fc domain.

The variant ABDEG™ Fc domain or FcRn binding fragment thereof of the anti-IgE antibodies described herein may be a variant Fc domain or FcRn binding fragment derived from any suitable wild-type immunoglobulin Fc domain. In certain embodiments, the variant ABDEG™ Fc domain or FcRn binding fragment thereof is a variant IgG Fc domain or FcRn binding fragment thereof. The wild-type IgG domain may be an IgG of any sub-class including IgG1, IgG2, IgG3 and IgG4. The wild-type IgG domain is preferably human.

In preferred embodiments, the variant ABDEG™ Fc domain or FcRn binding fragment thereof is a variant IgG1 Fc domain or FcRn binding fragment thereof. In such embodiments, the variant ABDEG™ Fc domain has the amino acid sequence of a wild-type IgG1 domain comprising or consisting of the ABDEG™ amino acid signature described herein, specifically amino acids Y, T, E, K, F and Y at EU positions 252, 254, 256, 433, 434 and 436, respectively. The wild-type IgG1 domain is preferably human.

In certain embodiments, the variant ABDEG™ Fc domain or FcRn binding fragment thereof consists of no more than 5, no more than 6, no more than 7, no more than 8, no more than 9, no more than 10, no more than 11, no more than 12, no more than 15, no more than 20 alterations as compared with the corresponding wild-type Fc domain. The alterations may be selected from amino acid substitutions, additions and/or deletions, linkage of additional moieties, and/or alteration of the native glycans. In certain embodiments, the variant ABDEG™ Fc domain or FcRn binding fragment thereof consists of no more than 5, no more than 6, no more than 7, no more than 8, no more than 9, no more than 10, no more than 11, no more than 12, no more than 15, no more than 20 amino acid substitutions as compared with the corresponding wild-type Fc domain.

In certain embodiments, the variant ABDEG™ Fc domain or FcRn binding fragment thereof comprises or consists of at least five amino acid substitutions but no more than 20 amino acid substitutions in total. In certain embodiments, the variant ABDEG™ Fc domain or FcRn binding fragment thereof comprises or consists of at least five amino acid substitutions but no more than amino acid substitutions in total.

In certain embodiments, the variant Fc domain or FcRn binding fragment is identical to the corresponding wild-type Fc domain or FcRn binding fragment but for the amino acids Y, T, E, K, F and Y at EU positions 252, 254, 256, 433, 434 and 436, respectively.

Non-limiting examples of variant Fc domains for inclusion in the anti-IgE antibodies described herein are set forth in Table 5 below. In certain embodiments, the variant Fc domain comprises or consists of the amino acid sequence set forth in SEQ ID NO: 1. In certain embodiments, the variant Fc domain comprises or consists of the amino acid sequence set forth in SEQ ID NO: 2.

In certain embodiments, the variant Fc domain comprises or consists of the amino acid sequence set forth in SEQ ID NO: 3. In certain embodiments, the variant Fc domain is linked to a heavy chain CH1 domain and the heavy chain constant region comprises or consists of the amino acid sequence set forth in SEQ ID NO: 4.

TABLE 5
Amino acid sequences of non-limiting examples
of variant Fc domains and heavy chain
constant regions incorporating variant Fc domains

| SEQ ID NO | Amino Acid Sequence |
|---|---|
| 1 | CPPCPAPELLGGPSVFLFPPKPKDTLYITREPEVTCVVVDVSHEDPEV |
| | KFNWYVDGVEVHNAKTKPREEQYNSTYRVVSVLTVLHQDWLNGKEY |
| | KCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKS |
| | RWQQGNVFSCSVMHEALKFHYTQKSLSLSPG |
| 2 | DKTHTCPPCPAPELLGGPSVFLFPPKPKDTLYITREPEVTCVVVDVSH |
| | EDPEVKFNWYVDGVEVHNAKTKPREEQYNSTYRVVSVLTVLHQDWL |
| | NGKEYKCKVSNKALPAPIEKTISKAKGQPREPQVYTLPPSRDELTKNQ |
| | VSLTCLVKGFYPSDIAVEWESNGQPENNYKTTPPVLDSDGSFFLYSKL |
| | TVDKSRWQQGNVFSCSVMHEALKFHYTQKSLSLSPGK |
| 3 | DKTHTCPPCPAPELLGGPSVFLFPPKPKDTLYITREPEVTCVVVDVSH |
| | EDPEVKFNWYVDGVEVHNAKTKPREEQYNSTYRVVSVLTVLHQDWL |
| | NGKEYKCKVSNKALPAPIEKTISKAKGQPREPQVYTLPPSRDELTKNQ |
| | VSLTCLVKGFYPSDIAVEWESNGQPENNYKTTPPVLDSDGSFFLYSKL |
| | TVDKSRWQQGNVFSCSVMHEALKFHYTQKSLSLSPG |
| 4 | ASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVD |
| | KKVEPKSCDKTHTCPPCPAPELLGGPSVFLFPPKPKDTLYITREPEVT |
| | CVVVDVSHEDPEVKFNWYVDGVEVHNAKTKPREEQYNSTYRVVSVL |
| | TVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSD |
| | GSFFLYSKLTVDKSRWQQGNVFSCSVMHEALKFHYTQKSLSLSPGK |
| 5 | CPPCPAPEAAGGPSVFLFPPKPKDTLYITREPEVTCVVVDVSHEDPEV |
| | KFNWYVDGVEVHNAKTKPREEQYNSTYRVVSVLTVLHQDWLNGKEY |
| | KCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKS |
| | RWQQGNVFSCSVMHEALKFHYTQKSLSLSPG |
| 6 | DKTHTCPPCPAPEAAGGPSVFLFPPKPKDTLYITREPEVTCVVVDVSH |
| | EDPEVKFNWYVDGVEVHNAKTKPREEQYNSTYRVVSVLTVLHQDWL |
| | NGKEYKCKVSNKALPAPIEKTISKAKGQPREPQVYTLPPSRDELTKNQ |
| | VSLTCLVKGFYPSDIAVEWESNGQPENNYKTTPPVLDSDGSFFLYSKL |
| | TVDKSRWQQGNVFSCSVMHEALKFHYTQKSLSLSPGK |
| 7 | DKTHTCPPCPAPEAAGGPSVFLFPPKPKDTLYITREPEVTCVVVDVSH |
| | EDPEVKFNWYVDGVEVHNAKTKPREEQYNSTYRVVSVLTVLHQDWL |
| | NGKEYKCKVSNKALPAPIEKTISKAKGQPREPQVYTLPPSRDELTKNQ |
| | VSLTCLVKGFYPSDIAVEWESNGQPENNYKTTPPVLDSDGSFFLYSKL |
| | TVDKSRWQQGNVFSCSVMHEALKFHYTQKSLSLSPG |
| 8 | ASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVD |
| | KKVEPKSCDKTHTCPPCPAPEAAGGPSVFLFPPKPKDTLYITREPEVT |
| | CVVVDVSHEDPEVKFNWYVDGVEVHNAKTKPREEQYNSTYRVVSVL |
| | TVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSD |
| | GSFFLYSKLTVDKSRWQQGNVFSCSVMHEALKFHYTQKSLSLSPGK |

ABDEG™ Amino acids at EU positions 252, 254, 256, 433, and 434 are shown in bold and underlined. Amino acids at positions 234 and 235 are underlined.
NB SEQ ID NOs: 1-3 and 5-7 represent variant Fc domains; SEQ ID NOs: 4 and 8 incorporate the CH1 domain sequence in addition to the variant Fc domain.

For embodiments wherein the variant Fc domain comprises one or more non-naturally occurring amino acid residues in addition to the ABDEG™ mutations, the variant Fc domain or FcRn binding fragment thereof may comprise the amino acids A, A at EU positions 234 and 235, respectively.

In certain embodiments, the variant Fc domain comprises or consists of the amino acid sequence set forth in SEQ ID NO: 5. In certain embodiments, the variant Fc domain comprises or consists of the amino acid sequence set forth in SEQ ID NO: 6. In certain embodiments, the variant Fc domain comprises or consists of the amino acid sequence set forth in SEQ ID NO: 7. In certain embodiments, the variant Fc domain is linked to a heavy chain CH1 domain and the heavy chain constant region comprises or consists of the amino acid sequence set forth in SEQ ID NO: 8.

As noted above, in certain embodiments, the anti-IgE antibodies of the invention comprise a variant Fc region consisting of two Fc domains or FcRn binding fragments thereof, wherein at least one of the Fc domains or FcRn binding fragments is a variant Fc domain or FcRn binding fragment as described herein. In certain embodiments, each of the two variant Fc domains or FcRn binding fragments of the variant Fc region comprise the amino acids Y, T, E, K, F and Y at EU positions 252, 254, 256, 433, 434 and 436, respectively. In certain embodiments, the two variant Fc domains of the variant Fc region are different and form a heterodimer. For heterodimeric embodiments, one or both of the Fc domains or FcRn binding fragments thereof may be a variant Fc domain or FcRn binding fragment. In alternative embodiments, the two variant Fc domains of the variant Fc region are identical and form a homodimer. In certain embodiments, the amino acid sequence of each of the variant Fc domains in the variant Fc region comprises or consists of the amino acid sequence set forth in SEQ ID NO: 1, 2 or 3. In certain embodiments, the amino acid sequence of each of the variant Fc domains in the variant Fc region comprises or consists of the amino acid sequence set forth in SEQ ID NO: 5, 6 or 7. For embodiments wherein the variant Fc domain comprises one or more non-naturally occurring amino acid residues in addition to the ABDEG™ mutations, the variant Fc domain or FcRn binding fragment thereof may comprise one or more additional Fc substitutions that have been reported to increase FcRn binding and thereby improve antibody pharmacokinetics. Such substitutions are reported in, for example, Zalevsky et al. (2010) Nat. Biotechnol. 28(2):157-9; Hinton et al. (2006) J Immunol. 176:346-356; Yeung et al. (2009) J Immunol. 182:7663-7671; Presta L G. (2008) Curr. Op. Immunol. 20:460-470; and Vaccaro et al. (2005) Nat. Biotechnol. 23(10):1283-88, the contents of which are incorporated herein in their entirety.

For embodiments wherein the variant Fc domain comprises one or more non-naturally occurring amino acid residues in addition to the ABDEG™ mutations, the variant Fc domain or FcRn binding fragment thereof may comprise a non-naturally occurring amino acid residue at one or more positions selected from the group consisting of 234, 235, 236, 239, 240, 241, 243, 244, 245, 247, 262, 263, 264, 265, 266, 267, 269, 296, 297, 298, 299, 313, 325, 326, 327, 328, 329, 330, 332, 333, and 334 as numbered by the EU index as set forth in Kabat. Optionally, the variant Fc domain may comprise a non-naturally occurring amino acid residue at additional and/or alternative positions known to one skilled in the art (see, e.g., U.S. Pat. Nos. 5,624,821; 6,277,375; 6,737,056; PCT Patent Publications WO 01/58957; WO 02/06919; WO 04/016750; WO 04/029207; WO 04/035752 and WO 05/040217, the contents of which are incorporated by reference herein in their entirety).

In certain embodiments, the variant Fc domain or FcRn binding fragment comprises at least one additional non-naturally occurring amino acid residue selected from the group consisting of 234D, 234E, 234N, 234Q, 234T, 234H, 234Y, 2341, 234V, 234F, 235A, 235D, 235R, 235W, 235P, 235S, 235N, 235Q, 235T, 235H, 235Y, 2351, 235V, 235F, 236E, 239D, 239E, 239N, 239Q, 239F, 239T, 239H, 239Y, 2401, 240A, 240T, 240M, 241W, 241 L, 241Y, 241E, 241R. 243W, 243L 243Y, 243R, 243Q, 244H, 245A, 247V, 247G, 2621, 262A, 262T, 262E, 2631, 263A, 263T, 263M, 264L, 2641, 264W, 264T, 264R, 264F, 264M, 264Y, 264E, 265G, 265N, 265Q, 265Y, 265F, 265V, 2651, 265L, 265H, 265T, 2661, 266A, 266T, 266M, 267Q, 267L, 269H, 269Y, 269F, 269R, 296E, 296Q, 296D, 296N, 296S, 296T, 296L, 2961, 296H, 269G, 297S, 297D, 297E, 298H, 298I, 298T, 298F, 2991, 299L, 299A, 299S, 299V, 299H, 299F, 299E, 313F, 325Q, 325L, 3251, 325D, 325E, 325A, 325T, 325V, 325H, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 3301, 330F, 330R, 330H, 332D, 332S, 332W, 332F, 332E, 332N, 332Q, 332T, 332H, 332Y, and 332A as numbered by the EU index as set forth in Kabat. Optionally, the Fc domain or FcRn binding fragment thereof may comprise additional and/or alternative non-naturally occurring amino acid residues known to one skilled in the art (see, e.g., U.S. Pat. Nos. 5,624,821; 6,277,375; 6,737,056; PCT Patent Publications WO 01/58957; WO 02/06919; WO 04/016750; WO 04/029207; WO 04/035752 and WO 05/040217, the contents of which are incorporated by reference herein in their entirety).

Additional Fc domain alterations that may be incorporated into the variant Fc domains or FcRn binding fragments also include without limitation those disclosed in Ghetie et al., 1997, Nat. Biotech. 15:637-40; Duncan et al, 1988, Nature 332:563-564; Lund et al., 1991, J. Immunol., 147:2657-2662; Lund et al, 1992, Mol. Immunol., 29:53-59; Alegre et al, 1994, Transplantation 57:1537-1543; Hutchins et al., 1995, Proc Natl. Acad Sci USA, 92:11980-11984; Jefferis et al, 1995, Immunol Lett., 44:111-117; Lund et al., 1995, Faseb J., 9:115-119; Jefferis et al, 1996, Immunol Lett., 54:101-104; Lund et al, 1996, J. Immunol., 157:4963-4969; Armour et al., 1999, Eur J Immunol. 29:2613-2624; Idusogie et al, 2000, J. Immunol., 164:4178-4184; Reddy et al, 2000, J. Immunol., 164:1925-1933; Xu et al., 2000, Cell Immunol., 200:16-26; Idusogie et al, 2001, J. Immunol., 166:2571-2575; Shields et al., 2001, J Biol. Chem., 276:6591-6604; Jefferis et al, 2002, Immunol Lett., 82:57-65; Presta et al., 2002, Biochem Soc Trans., 30:487-490); U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 6,821,505; 6,277,375; U.S. Patent Publication Nos. 2004/0002587 and PCT Publications WO 94/29351; WO 99/58572; WO 00/42072; WO 02/060919; WO 04/029207; WO 04/099249; WO 04/063351, the contents of which are incorporated by reference herein in their entirety.

As described herein, the variant Fc domains or FcRn binding fragments thereof incorporated into the anti-IgE antibodies of the present invention can aid in the clearance of pathogenic IgG autoantibodies from the body. This effect is mediated by the higher-affinity binding of the anti-IgE antibodies to the FcRn receptor as effected by the variant Fc domain(s) or FcRn binding fragments thereof. It is believed that pathogenic IgG antibodies observed in autoimmune diseases are either the pathogenic triggers for these diseases or contribute to disease progression and mediate disease through the inappropriate activation of cellular Fc receptors. Aggregated autoantibodies and/or autoantibodies complexed with self-antigens (immune complexes) bind to activating Fc receptors, causing numerous autoimmune diseases (which occur in part because of immunologically mediated inflammation against self-tissues) (see e.g., Clarkson et al., NEJM 314(9), 1236-1239 (2013)); US20040010124A1; US20040047862A1; and US2004/0265321A1, incorporated herein by reference in their entirety).

Accordingly, to treat antibody-mediated disorders (e.g. autoimmune diseases), it would be advantageous to both remove the deleterious autoantibodies and to block the interaction of the immune complexes of these antibodies with activating Fc receptors (e.g., Fcγ receptors, such as CD16a). Accordingly, in certain embodiments, the variant Fc domain or variant Fc region of the anti-IgE antibody exhibits increased binding to CD16a (e.g., human CD16a). This is particularly advantageous in that it allows the anti-IgE antibody to additionally antagonize the immune complex-induced inflammatory response of autoantibodies being targeted for removal by FcRn inhibition. Any art recognized means of increasing affinity for CD16a (e.g., human CD16a) can be employed. In certain embodiments, the anti-IgE antibody comprises a variant Fc domain or variant Fc-region comprising an N-linked glycan (e.g., at EU position 297). In this case it is possible to increase the binding affinity of the anti-IgE antibody for CD16a by altering the glycan structure. Alterations of the N-linked glycan of Fc regions are well known in the art. For example, afucosylated N-linked glycans or N-glycans having a bisecting GlcNac structure have been shown to exhibit increased affinity for CD16a. Accordingly, in certain embodiments, the N-linked glycan is afucosylated. Afucosylation can be achieved using any art recognized means. For example, an anti-IgE antibody can be expressed in cells lacking fucosyl transferase, such that fucose is not added to the N-linked glycan at EU position 297 of the variant Fc domain or variant Fc region (see e.g., U.S. Pat. No. 8,067,232, the contents of which is incorporated by reference herein in its entirety). In certain embodiments, the N-linked glycan has a bisecting GlcNac structure. The bisecting GlcNac structure can be achieved using any art recognized means. For example, an anti-IgE antibody can be expressed in cells expressing beta1-4-N-acetylglucosaminyltransferase III (GnTIII), such that bisecting GlcNac is added to the N-linked glycan at EU position 297 of the variant Fc domain or variant Fc region (see e.g., U.S. Pat. No. 8,021,856, the contents of which is incorporated by reference herein in its entirety). Additionally or alternatively, alterations of the N-linked glycan structure can also be achieved by enzymatic means in vitro.

To enhance the manufacturability of the IgE antibodies of the present invention disclosed herein, it is preferable that the variant Fc domains or variant Fc regions do not comprise any non-disulphide bonded cysteine residues. Accordingly, in certain embodiments the variant Fc domains or variant Fc regions do not comprise a free cysteine residue.

In certain embodiments, the variant Fc domain or variant Fc region has altered (e.g., increased or decreased) binding affinity for an additional Fc receptor. The variant Fc domain or variant Fc region can have altered (e.g., increased or decreased) binding affinity for one or more of Fcγ receptors e.g., FcγRI (CD64), FcγRIIA (CD32), FcγRIIB (CD32), FcγRIIIA (CD16a), and FcγRIIIB (CD16b). Any art recognized means of altering the affinity for an additional Fc receptor can be employed.

(iii) Antibodies that Bind IgE

The anti-IgE antibodies of the present invention may adopt the format of any suitable antibody displaying immunoreactivity for IgE, provided that the antibody comprises at least one variant Fc domain or FcRn binding fragment as described above. In this regard, the term “antibody” should be construed broadly so as to encompass bivalent tetrameric antibodies, including humanized and germlined variants thereof, and also modified antibodies having a non-native immunoglobulin structure.

The anti-IgE antibodies of the invention may comprise, in addition to the variant Fc domain or FcRn binding fragment thereof described above, any antigen-binding fragment or region. In certain embodiments, said antigen-binding fragment or region comprises or consists of a VH-VL domain pairing, a scFv fragment, a Fab, a Fab′, a F(ab′)2. In certain embodiments, the anti-IgE antibody is a bivalent IgG having a variant Fc region or FcRn binding fragment as defined herein. In certain embodiments, the anti-IgE antibody is a monovalent IgG having a variant Fc domain or FcRn binding fragment as defined herein. Monovalent anti-IgE antibodies may be advantageous in that they may not have the ability to cross-link FcεRI receptors.

The antibodies described herein are intended for human therapeutic use and therefore, will typically be of the IgA, IgD, IgE, IgG, IgM type, often of the IgG type, in which case they can belong to any of the four sub-classes IgG1, IgG2a and b, IgG3 or IgG4. In preferred embodiments, the anti-IgE antibodies of the invention are IgG antibodies, optionally IgG1 antibodies. The antibodies may be monoclonal, polyclonal, multispecific (e.g. bispecific antibodies) antibodies, provided that they exhibit the appropriate immunological specificity for their target. Monoclonal antibodies are preferred since they are highly specific, being directed against a single antigenic site.

The anti-IgE antibodies described herein may exhibit high human homology. Such antibody molecules having high human homology may include antibodies comprising VH and VL domains of native non-human antibodies which exhibit sufficiently high % sequence identity to human germline sequences. In certain embodiments, the antibody molecules are humanised or germlined variants of non-human antibodies.

The anti-IgE antibodies described herein preferably inhibit the binding of IgE to its receptor, FcεRI. In certain embodiments, the anti-IgE antibodies inhibit binding of IgE to both FcεRI and FcεRII. The anti-IgE antibodies may bind to an epitope located within the CH3 domain of the IgE heavy chain. The anti-IgE antibodies described herein preferably do not bind to IgE that is already associated with FcεRI i.e. membrane-localised IgE. In preferred embodiments, the anti-IgE antibodies of the invention are not anaphylactic.

### (iv) PH-Dependent Antibodies

Any of the anti-IgE antibodies described herein may exhibit pH-dependent antigen binding i.e. pH-dependent binding to IgE.

Antibodies that have bound antigen are taken up into cells and trafficked to the endosomal-lysosomal degradation pathway. Antibodies that are able to dissociate from their antigen in the early endosome can be recycled back to the cell surface. Antibodies that bind with high affinity to their antigen in the endosomal compartments are typically trafficked to the lysosomes for degradation. It has been shown previously that if an antibody has pH-dependent antigen binding activity, such that it has a lower binding affinity for its antigen at early endosomal pH as compared with plasma pH, the antibody will recycle to the cell surface more efficiently. This can extend the antibody plasma half-life and allow the same antibody to bind to multiple antigens. For this reason, it is advantageous for the anti-IgE antibodies described herein to exhibit pH-dependent antigen binding. pH-dependent anti-IgE antibodies in accordance with the present invention have the potential to eliminate serum IgE autoantibodies by binding to these autoantibodies in the circulation and internalising the IgE autoantibodies. The IgE autoantibodies may be released in the acidic endosomal compartment and trafficked to the lysosomes for degradation. The free anti-IgE antibodies of the invention may be recycled to the cell surface such that they can bind and internalise further IgE autoantibodies.

The anti-IgE antibodies of the invention may possess intrinsic pH-dependent antigen binding activity i.e. they may have been selected for this property. Alternatively or in addition, the anti-IgE antibodies described herein may be engineered so as to exhibit pH-dependent target binding. Methods of engineering pH-dependent antigen binding activity in antibody molecules are described in, for example, EP2275443, which is incorporated herein by reference. Methods of engineering pH-dependent antigen binding in antibody molecules are also described in WO2018/206748, which is incorporated herein by reference. The antibodies described herein may be modified by any technique so as to achieve pH-dependent binding. For example, the antibodies may be modified in accordance with the methods described in EP2275443 or WO2018/206748 such that they exhibit pH-dependent antigen binding.

For pH-dependent embodiments of the anti-IgE antibodies described herein, the antigen-binding activity is lower at endosomal pH as compared to the antigen-binding activity at plasma pH. The endosomal pH is typically acidic pH whereas the plasma pH is typically neutral pH. Accordingly, the antibodies described herein, may exhibit pH-dependent antigen binding such that their antigen-binding activity is lower at acidic pH as compared to the antigen-binding activity at neutral pH. Endosomal pH or “acidic pH” may be pH of from about pH 4.0 to about pH 6.5, preferably from about pH 5.5 to about pH 6.5, preferably from about pH 5.5 to about pH 6.0, preferably pH 5.5, pH 5.6, pH 5.7 or pH 5.8. Plasma pH or “neutral pH” may be pH of from about pH 6.9 to about pH 8.0, preferably from about pH 7.0 to about pH 8.0, preferably from about pH 7.0 to about pH 7.4, preferably pH 7.0 or pH 7.4.

In certain embodiments, the anti-IgE antibodies exhibit pH-dependent binding such that the antigen-binding activity at pH 5.8 is lower as compared with the antigen-binding activity at pH 7.4. The pH-dependent anti-IgE antibodies may be characterised in that the dissociation constant (KD) for the antibody-antigen interaction at acidic pH or pH 5.8 is higher than the dissociation constant (KD) for the antibody-antigen interaction at neutral pH or at pH 7.4. In certain embodiments, the anti-IgE antibodies exhibit pH-dependent binding such that the ratio of KD for the antigen at pH 5.8 and KD for the antigen at pH 7.4 (KD(pH5.8)/KD(pH7.4)) is 2 or more, 4 or more, 6 or more, 8 or more, 10 or more, 12 or more.

The pH-dependent antigen-binding activity of an antibody molecule may be engineered by modifying an antibody molecule so as to impair the antigen-binding ability at acidic pH and/or increase the antigen-binding ability at neutral pH. For example, the antibody molecule may be modified by substituting at least one amino acid of the antibody molecule with histidine, or by inserting at least one histidine into the antibody molecule. Such histidine mutation (substitution or insertion) sites are not particularly limited, and any site is acceptable as long as the antigen-binding activity at endosomal pH (for example pH 5.8) is lower than that at plasma pH (for example pH 7.4) as compared to before the mutation or insertion.

In certain embodiments, the anti-IgE antibodies may be engineered so as to exhibit pH-dependent antigen binding by the introduction of one or more substitutions into the variable domains. In preferred embodiments, the anti-IgE antibodies are engineered so as to exhibit pH-dependent antigen binding by introducing one or more substitutions into one or more CDRs of the antibody. The substitutions may introduce one or more His residues into one or more sites of the variable domains, preferably the heavy chain and/or light chain CDRs so as to confer pH-dependent antigen binding.

For embodiments of the invention wherein the antibody comprises three heavy chain CDR sequences and three light chain CDR sequences, the six CDRs combined may consist of a total of 1-10 His substitutions, optionally 1-5 His substitutions, optionally 1, 2, 3, 4, 5, 6, 7, 8, 9 or 10 His substitutions. The anti-IgE antibodies may be engineered in accordance with the methods described in WO2018/206748, incorporated herein by reference. Non-histidine substitutions may also be incorporated into variable domains, particularly the CDRs, of the pH-dependent antibodies described herein.

In preferred embodiments, the exemplary anti-IgE antibodies having the particular CDR, VH and/or VL domain sequences recited herein are engineered such that they exhibit pH-dependent antigen binding. For example, the CDR sequences of the exemplary anti-IgE antibodies described herein may be modified by the introduction of one or more Histidine substitutions so as to produce antibodies exhibiting pH-dependent antigen binding.

### (v) Camelid-Derived Anti-IgE Antibodies

The anti-IgE antibodies of the present invention may be camelid-derived. Camelid-derived antibodies may be heavy-chain only antibodies i.e. VHH antibodies or may be conventional heterotetrameric antibodies. In preferred embodiments, the anti-IgE antibodies of the invention are derived from camelid heterotetrameric antibodies.

For example, the antibody molecules may be selected from immune libraries obtained by a method comprising the step of immunizing a camelid with IgE, preferably human IgE. The camelid may be immunized with IgE protein or a polypeptide fragment thereof, or with an mRNA molecule or cDNA molecule expressing the protein or polypeptide fragment thereof. Methods for producing antibodies in camelid species and selecting antibodies against preferred targets from camelid immune libraries are described in, for example, International patent application no. WO2010/001251, incorporated herein by reference.

In certain embodiments, the antibody molecules may be camelid-derived in that they comprise at least one hypervariable loop or complementarity determining region obtained from a VH domain or a VL domain of a species in the family Camelidae. In particular, the antibody molecule may comprise VH and/or VL domains, or CDRs thereof, obtained by active immunisation of outbred camelids, e.g. llamas, with IgE.

The term “obtained from” in this context implies a structural relationship, in the sense that the HVs or CDRs of the antibody molecule embody an amino acid sequence (or minor variants thereof) which was originally encoded by a Camelidae immunoglobulin gene. However, this does not necessarily imply a particular relationship in terms of the production process used to prepare the antibody molecule.

Camelid-derived antibody molecules may be derived from any camelid species, including inter alia, llama, dromedary, alpaca, vicuna, guanaco or camel.

Antibody molecules comprising camelid-derived VH and VL domains, or CDRs thereof, are typically recombinantly expressed polypeptides, and may be chimeric polypeptides. The term “chimeric polypeptide” refers to an artificial (non-naturally occurring) polypeptide which is created by juxtaposition of two or more peptide fragments which do not otherwise occur contiguously.

Included within this definition are “species” chimeric polypeptides created by juxtaposition of peptide fragments encoded by two or more species, e.g. camelid and human.

In certain embodiments, the entire VH domain and/or the entire VL domain may be obtained from a species in the family Camelidae. The camelid-derived VH domain and/or the camelid-derived VL domain may then be subject to protein engineering, in which one or more amino acid substitutions, insertions or deletions are introduced into the camelid amino acid sequence. These engineered changes preferably include amino acid substitutions relative to the camelid sequence. Such changes include “humanisation” or “germlining” wherein one or more amino acid residues in a camelid-encoded VH or VL domain are replaced with equivalent residues from a homologous human-encoded VH or VL domain.

Isolated camelid VH and VL domains obtained by active immunisation of a camelid (e.g. llama) can be used as a basis for engineering antibody molecules in accordance with the invention. Starting from intact camelid VH and VL domains, it is possible to engineer one or more amino acid substitutions, insertions or deletions which depart from the starting camelid sequence. In certain embodiments, such substitutions, insertions or deletions may be present in the framework regions of the VH domain and/or the VL domain.

In other embodiments, there are provided “chimeric” antibody molecules comprising camelid-derived VH and VL domains (or engineered variants thereof) and one or more constant domains from a non-camelid antibody, for example human-encoded constant domains (or engineered variants thereof). In such embodiments it is preferred that both the VH domain and the VL domain are obtained from the same species of camelid, for example both VH and VL may be from Lama glama or both VH and VL may be from Lama pacos (prior to introduction of engineered amino acid sequence variation). In such embodiments both the VH and the VL domain may be derived from a single animal, particularly a single animal which has been actively immunised with the antigen of interest.

As an alternative to engineering changes in the primary amino acid sequence of Camelidae VH and/or VL domains, individual camelid-derived hypervariable loops or CDRs, or combinations thereof, can be isolated from camelid VH/VL domains and transferred to an alternative (i.e. non-Camelidae) framework, e.g. a human VH/VL framework, by CDR grafting.

In non-limiting embodiments, the anti-IgE antibody molecules of the invention may comprise CH1 domains and/or CL domains (from the heavy chain and light chain, respectively), the amino acid sequence of which is fully or substantially human. For antibody molecules intended for human therapeutic use, it is typical for the entire constant region of the antibody, or at least a part thereof, to have fully or substantially human amino acid sequence. As described herein, the variant Fc domains and/or variant Fc regions of the anti-IgE antibodies of the invention may be variant human Fc domains and/or variant human Fc regions. The CDRs or antigen-binding domains of camelid-derived IgE antibodies, including humanized and germlined variants thereof, may be combined with any of the variant human Fc domains or variant human Fc regions as described in sections (i) and (ii) above.

One or more or any combination of the CH1 domain, hinge region, CH2 domain, CH3 domain and CL domain (and CH4 domain if present) may be fully or substantially human with respect to its amino acid sequence. The CH1 domain, hinge region, CH2 domain, CH3 domain and/or CL domain (and/or CH4 domain if present) may be derived from a human antibody, preferably a human IgG antibody, more preferably a human IgG1 antibody of subtype IgG1, IgG2, IgG3 or IgG4. As described herein, the variant Fc domains and variant Fc regions of the anti-IgE antibodies of the invention may be variant human IgG Fc domains or variant human IgG Fc regions, for example variant human IgG1, IgG2, IgG3 or IgG4 Fc domains or Fc regions. The CDRs or antigen-binding domains of camelid-derived IgE antibodies, including humanized and germlined variants thereof, may be combined with any of the variant human Fc IgG domains or variant human IgG Fc regions as described in sections (i) and (ii) above.

Advantageously, the CH1 domain, hinge region, CH2 domain, CH3 domain and CL domain (and CH4 domain if present) may all have substantially human amino acid sequence. In the context of the constant region of a humanised or chimeric antibody, or an antibody fragment, the term “substantially human” refers to an amino acid sequence identity of at least 90%, or at least 92%, or at least 95%, or at least 97%, or at least 99% with a human constant region. The term “human amino acid sequence” in this context refers to an amino acid sequence which is encoded by a human immunoglobulin gene, which includes germline, rearranged and somatically mutated genes.

### (vi) Exemplary Camelid-Derived Anti-IgE Antibodies

In certain embodiments, the anti-IgE antibodies of the invention are selected from antibodies comprising a combination of variable heavy chain CDR3 (HCDR3), variable heavy chain CDR2 (HCDR2) and variable heavy chain CDR1 (HCDR1), variable light chain CDR3 (LCDR3), variable light chain CDR2 (LCDR2) and variable light chain CDR1 (LCDR1) selected from the following:
(i) HCDR3 comprising SEQ ID NO: 11; HCDR2 comprising SEQ ID NO: 10; HCDR1 comprising SEQ ID NO: 9; LCDR3 comprising SEQ ID NO: 56; LCDR2 comprising SEQ ID NO: 55; and LCDR1 comprising SEQ ID NO: 54;
(ii) HCDR3 comprising SEQ ID NO: 14; HCDR2 comprising SEQ ID NO: 13; HCDR1 comprising SEQ ID NO: 12; LCDR3 comprising SEQ ID NO: 58; LCDR2 comprising SEQ ID NO: 55; and LCDR1 comprising SEQ ID NO: 57;
(iii) HCDR3 comprising SEQ ID NO: 17; HCDR2 comprising SEQ ID NO: 16; HCDR1 comprising SEQ ID NO: 15; LCDR3 comprising SEQ ID NO: 61; LCDR2 comprising SEQ ID NO: 60; and LCDR1 comprising SEQ ID NO: 59;
(iv) HCDR3 comprising SEQ ID NO: 19; HCDR2 comprising SEQ ID NO: 18; HCDR1 comprising SEQ ID NO: 12; LCDR3 comprising SEQ ID NO: 61; LCDR2 comprising SEQ ID NO: 60; and LCDR1 comprising SEQ ID NO: 59;
(v) HCDR3 comprising SEQ ID NO: 22; HCDR2 comprising SEQ ID NO: 21; HCDR1 comprising SEQ ID NO: 20; LCDR3 comprising SEQ ID NO: 63; LCDR2 comprising SEQ ID NO: 55; and LCDR1 comprising SEQ ID NO: 62;
(vi) HCDR3 comprising SEQ ID NO: 24; HCDR2 comprising SEQ ID NO: 23; HCDR1 comprising SEQ ID NO: 20; LCDR3 comprising SEQ ID NO: 66; LCDR2 comprising SEQ ID NO: 65; and LCDR1 comprising SEQ ID NO: 64;
(vii) HCDR3 comprising SEQ ID NO: 27; HCDR2 comprising SEQ ID NO: 26; HCDR1 comprising SEQ ID NO: 25; LCDR3 comprising SEQ ID NO: 66; LCDR2 comprising SEQ ID NO: 67; and LCDR1 comprising SEQ ID NO: 54;
(viii) HCDR3 comprising SEQ ID NO: 22; HCDR2 comprising SEQ ID NO: 21; HCDR1 comprising SEQ ID NO: 20; LCDR3 comprising SEQ ID NO: 56; LCDR2 comprising SEQ ID NO: 69; and LCDR1 comprising SEQ ID NO: 68;
(ix) HCDR3 comprising SEQ ID NO: 30; HCDR2 comprising SEQ ID NO: 29; HCDR1 comprising SEQ ID NO: 28; LCDR3 comprising SEQ ID NO: 72; LCDR2 comprising SEQ ID NO: 71; and LCDR1 comprising SEQ ID NO: 70;
(x) HCDR3 comprising SEQ ID NO: 33; HCDR2 comprising SEQ ID NO: 32; HCDR1 comprising SEQ ID NO: 31; LCDR3 comprising SEQ ID NO: 56; LCDR2 comprising SEQ ID NO: 55; and LCDR1 comprising SEQ ID NO: 54;
(xi) HCDR3 comprising SEQ ID NO: 22; HCDR2 comprising SEQ ID NO: 23; HCDR1 comprising SEQ ID NO: 34; LCDR3 comprising SEQ ID NO: 63; LCDR2 comprising SEQ ID NO: 55; and LCDR1 comprising SEQ ID NO: 62;
(xii) HCDR3 comprising SEQ ID NO: 37; HCDR2 comprising SEQ ID NO: 36; HCDR1 comprising SEQ ID NO: 35; LCDR3 comprising SEQ ID NO: 75; LCDR2 comprising SEQ ID NO: 74; and LCDR1 comprising SEQ ID NO: 73;
(xiii) HCDR3 comprising SEQ ID NO: 38; HCDR2 comprising SEQ ID NO: 21; HCDR1 comprising SEQ ID NO: 20; LCDR3 comprising SEQ ID NO: 63; LCDR2 comprising SEQ ID NO: 55; and LCDR1 comprising SEQ ID NO: 62;
(xiv) HCDR3 comprising SEQ ID NO: 40; HCDR2 comprising SEQ ID NO: 39; HCDR1 comprising SEQ ID NO: 12; LCDR3 comprising SEQ ID NO: 78; LCDR2 comprising SEQ ID NO: 77; and LCDR1 comprising SEQ ID NO: 76;
(xv) HCDR3 comprising SEQ ID NO: 43; HCDR2 comprising SEQ ID NO: 42; HCDR1 comprising SEQ ID NO: 41; LCDR3 comprising SEQ ID NO: 81; LCDR2 comprising SEQ ID NO: 80; and LCDR1 comprising SEQ ID NO: 79;
(xvi) HCDR3 comprising SEQ ID NO: 14; HCDR2 comprising SEQ ID NO: 13; HCDR1 comprising SEQ ID NO: 12; LCDR3 comprising SEQ ID NO: 56; LCDR2 comprising SEQ ID NO: 55; and LCDR1 comprising SEQ ID NO: 82;
(xvii) HCDR3 comprising SEQ ID NO: 45; HCDR2 comprising SEQ ID NO: 44; HCDR1 comprising SEQ ID NO: 12; LCDR3 comprising SEQ ID NO: 66; LCDR2 comprising SEQ ID NO: 55; and LCDR1 comprising SEQ ID NO: 54;
(xviii) HCDR3 comprising SEQ ID NO: 48; HCDR2 comprising SEQ ID NO: 47; HCDR1 comprising SEQ ID NO: 46; LCDR3 comprising SEQ ID NO: 85; LCDR2 comprising SEQ ID NO: 84; and LCDR1 comprising SEQ ID NO: 83;
(xix) HCDR3 comprising SEQ ID NO: 50; HCDR2 comprising SEQ ID NO: 49; HCDR1 comprising SEQ ID NO: 12; LCDR3 comprising SEQ ID NO: 88; LCDR2 comprising SEQ ID NO: 87; and LCDR1 comprising SEQ ID NO: 86; and
(xx) HCDR3 comprising SEQ ID NO: 53; HCDR2 comprising SEQ ID NO: 52; HCDR1 comprising SEQ ID NO: 51; LCDR3 comprising SEQ ID NO: 91; LCDR2 comprising SEQ ID NO: 90; and LCDR1 comprising SEQ ID NO: 89.

In certain embodiments, the anti-IgE antibodies of the invention are selected from antibodies comprising a combination of variable heavy chain CDR3 (HCDR3), variable heavy chain CDR2 (HCDR2) and variable heavy chain CDR1 (HCDR1), variable light chain CDR3 (LCDR3), variable light chain CDR2 (LCDR2) and variable light chain CDR1 (LCDR1) selected from the following:
(i) HCDR3 comprising SEQ ID NO: 22; HCDR2 comprising SEQ ID NO: 21; HCDR1 comprising SEQ ID NO: 132; LCDR3 comprising SEQ ID NO: 56; LCDR2 comprising SEQ ID NO: 69; and LCDR1 comprising SEQ ID NO: 68;
(ii) HCDR3 comprising SEQ ID NO: 22; HCDR2 comprising SEQ ID NO: 21; HCDR1 comprising SEQ ID NO: 20; LCDR3 comprising SEQ ID NO: 56; LCDR2 comprising SEQ ID NO: 69; and LCDR1 comprising SEQ ID NO: 135;
(iii) HCDR3 comprising SEQ ID NO: 22; HCDR2 comprising SEQ ID NO: 21; HCDR1 comprising SEQ ID NO: 132; LCDR3 comprising SEQ ID NO: 56; LCDR2 comprising SEQ ID NO: 69; and LCDR1 comprising SEQ ID NO: 135;
(iv) HCDR3 comprising SEQ ID NO: 24; HCDR2 comprising SEQ ID NO: 23; HCDR1 comprising SEQ ID NO: 133; LCDR3 comprising SEQ ID NO: 66; LCDR2 comprising SEQ ID NO: 65; and LCDR1 comprising SEQ ID NO: 64;
(v) HCDR3 comprising SEQ ID NO: 24; HCDR2 comprising SEQ ID NO: 23; HCDR1 comprising SEQ ID NO: 20; LCDR3 comprising SEQ ID NO: 66; LCDR2 comprising SEQ ID NO: 65; and LCDR1 comprising SEQ ID NO: 64;
(vi) HCDR3 comprising SEQ ID NO: 19; HCDR2 comprising SEQ ID NO: 18; HCDR1 comprising SEQ ID NO: 134; LCDR3 comprising SEQ ID NO: 61; LCDR2 comprising SEQ ID NO: 60; and LCDR1 comprising SEQ ID NO: 59; and
(vii) HCDR3 comprising SEQ ID NO: 19; HCDR2 comprising SEQ ID NO: 18; HCDR1 comprising SEQ ID NO: 12; LCDR3 comprising SEQ ID NO: 136; LCDR2 comprising SEQ ID NO: 60; and LCDR1 comprising SEQ ID NO: 59.

In preferred embodiments, the anti-IgE antibodies of the invention comprise:
a variable heavy chain CDR3 comprising or consisting of SEQ ID NO: 22 [[GTSYSGSYYYTDPFFGS];
a variable heavy chain CDR2 comprising or consisting of SEQ ID NO: 21 [SIYHDGSHTYYADFVKG];
a variable heavy chain CDR1 comprising or consisting of SEQ ID NO: 132 [SYVMH];
a variable light chain CDR3 comprising or consisting of SEQ ID NO: 56 [QSADSSGNPV];
a variable light chain CDR2 comprising or consisting of SEQ ID NO: 69 [DDDRRPS]; and
a variable light chain CDR1 comprising or consisting of SEQ ID NO: 135 [QGDRLGSRYIH].

In preferred embodiments, the anti-IgE antibodies of the invention comprise:
a variable heavy chain CDR3 comprising SEQ ID NO: 22 [GTSYSGSYYYTDPFFGS];
a variable heavy chain CDR2 comprising SEQ ID NO: 21 [SIYHDGSHTYYADFVKG];
a variable heavy chain CDR1 comprising SEQ ID NO: 20 [SYVMS];
a variable light chain CDR3 comprising SEQ ID NO: 56 [QSADSSGNPV];
a variable light chain CDR2 comprising SEQ ID NO: 69 [DDDRRPS]; and a variable light chain CDR1 comprising SEQ ID NO: 135 [QGDRLGSRYIH].

In certain embodiments, the anti-IgE antibodies are selected from antibodies comprising a variable heavy chain domain (VH) and a variable light chain domain (VL) selected from the following:
(i) a VH domain comprising or consisting of the amino acid sequence of SEQ ID NO: 92 or an amino acid sequence having at least 80%, 90%, 95%, 98% 99% identity thereto, and a VL domain comprising or consisting of the amino acid sequence of SEQ ID NO: 93 or an amino acid sequence having at least 80%, 90%, 95%, 98% 99% identity thereto;
(ii) a VH domain comprising or consisting of the amino acid sequence of SEQ ID NO: 94 or an amino acid sequence having at least 80%, 90%, 95%, 98% 99% identity thereto, and a VL domain comprising or consisting of the amino acid sequence of SEQ ID NO: 95 or an amino acid sequence having at least 80%, 90%, 95%, 98% 99% identity thereto;
(iii) a VH domain comprising or consisting of the amino acid sequence of SEQ ID NO: 96 or an amino acid sequence having at least 80%, 90%, 95%, 98% 99% identity thereto, and a VL domain comprising or consisting of the amino acid sequence of SEQ ID NO: 97 or an amino acid sequence having at least 80%, 90%, 95%, 98% 99% identity thereto;
(iv) a VH domain comprising or consisting of the amino acid sequence of SEQ ID NO: 98 or an amino acid sequence having at least 80%, 90%, 95%, 98% 99% identity thereto, and a VL domain comprising or consisting of the amino acid sequence of SEQ ID NO: 99 or an amino acid sequence having at least 80%, 90%, 95%, 98% 99% identity thereto;
(v) a VH domain comprising or consisting of the amino acid sequence of SEQ ID NO: 100 or an amino acid sequence having at least 80%, 90%, 95%, 98% 99% identity thereto, and a VL domain comprising or consisting of the amino acid sequence of SEQ ID NO: 101 or an amino acid sequence having at least 80%, 90%, 95%, 98% 99% identity thereto;
(vi) a VH domain comprising or consisting of the amino acid sequence of SEQ ID NO: 102 or an amino acid sequence having at least 80%, 90%, 95%, 98% 99% identity thereto, and a VL domain comprising or consisting of the amino acid sequence of SEQ ID NO: 103 or an amino acid sequence having at least 80%, 90%, 95%, 98% 99% identity thereto;
(vii) a VH domain comprising or consisting of the amino acid sequence of SEQ ID NO: 104 or an amino acid sequence having at least 80%, 90%, 95%, 98% 99% identity thereto, and a VL domain comprising or consisting of the amino acid sequence of SEQ ID NO: 105 or an amino acid sequence having at least 80%, 90%, 95%, 98% 99% identity thereto;
(viii) a VH domain comprising or consisting of the amino acid sequence of SEQ ID NO: 106 or an amino acid sequence having at least 80%, 90%, 95%, 98% 99% identity thereto, and a VL domain comprising or consisting of the amino acid sequence of SEQ ID NO: 107 or an amino acid sequence having at least 80%, 90%, 95%, 98% 99% identity thereto;
(ix) a VH domain comprising or consisting of the amino acid sequence of SEQ ID NO: 108 or an amino acid sequence having at least 80%, 90%, 95%, 98% 99% identity thereto, and a VL domain comprising or consisting of the amino acid sequence of SEQ ID NO: 109 or an amino acid sequence having at least 80%, 90%, 95%, 98% 99% identity thereto;
(x) a VH domain comprising or consisting of the amino acid sequence of SEQ ID NO: 110 or an amino acid sequence having at least 80%, 90%, 95%, 98% 99% identity thereto, and a VL domain comprising or consisting of the amino acid sequence of SEQ ID NO: 111 or an amino acid sequence having at least 80%, 90%, 95%, 98% 99% identity thereto;
(xi) a VH domain comprising or consisting of the amino acid sequence of SEQ ID NO: 112 or an amino acid sequence having at least 80%, 90%, 95%, 98% 99% identity thereto, and a VL domain comprising or consisting of the amino acid sequence of SEQ ID NO: 113 or an amino acid sequence having at least 80%, 90%, 95%, 98% 99% identity thereto;
(xii) a VH domain comprising or consisting of the amino acid sequence of SEQ ID NO: 114 or an amino acid sequence having at least 80%, 90%, 95%, 98% 99% identity thereto, and a VL domain comprising or consisting of the amino acid sequence of SEQ ID NO: 115 or an amino acid sequence having at least 80%, 90%, 95%, 98% 99% identity thereto;
(xiii) a VH domain comprising or consisting of the amino acid sequence of SEQ ID NO: 116 or an amino acid sequence having at least 80%, 90%, 95%, 98% 99% identity thereto, and a VL domain comprising or consisting of the amino acid sequence of SEQ ID NO: 117 or an amino acid sequence having at least 80%, 90%, 95%, 98% 99% identity thereto;
(xiv) a VH domain comprising or consisting of the amino acid sequence of SEQ ID NO: 118 or an amino acid sequence having at least 80%, 90%, 95%, 98% 99% identity thereto, and a VL domain comprising or consisting of the amino acid sequence of SEQ ID NO: 119 or an amino acid sequence having at least 80%, 90%, 95%, 98% 99% identity thereto;
(xv) a VH domain comprising or consisting of the amino acid sequence of SEQ ID NO: 120 or an amino acid sequence having at least 80%, 90%, 95%, 98% 99% identity thereto, and a VL domain comprising or consisting of the amino acid sequence of SEQ ID NO: 121 or an amino acid sequence having at least 80%, 90%, 95%, 98% 99% identity thereto;
(xvi) a VH domain comprising or consisting of the amino acid sequence of SEQ ID NO: 122 or an amino acid sequence having at least 80%, 90%, 95%, 98% 99% identity thereto, and a VL domain comprising or consisting of the amino acid sequence of SEQ ID NO: 123 or an amino acid sequence having at least 80%, 90%, 95%, 98% 99% identity thereto;
(xvii) a VH domain comprising or consisting of the amino acid sequence of SEQ ID NO: 124 or an amino acid sequence having at least 80%, 90%, 95%, 98% 99% identity thereto, and a VL domain comprising or consisting of the amino acid sequence of SEQ ID NO: 125 or an amino acid sequence having at least 80%, 90%, 95%, 98% 99% identity thereto;
(xviii) a VH domain comprising or consisting of the amino acid sequence of SEQ ID NO: 126 or an amino acid sequence having at least 80%, 90%, 95%, 98% 99% identity thereto, and a VL domain comprising or consisting of the amino acid sequence of SEQ ID NO: 127 or an amino acid sequence having at least 80%, 90%, 95%, 98% 99% identity thereto;
(xix) a VH domain comprising or consisting of the amino acid sequence of SEQ ID NO: 128 or an amino acid sequence having at least 80%, 90%, 95%, 98% 99% identity thereto, and a VL domain comprising or consisting of the amino acid sequence of SEQ ID NO: 129 or an amino acid sequence having at least 80%, 90%, 95%, 98% 99% identity thereto; and
(xx) a VH domain comprising or consisting of the amino acid sequence of SEQ ID NO: 130 or an amino acid sequence having at least 80%, 90%, 95%, 98% 99% identity thereto, and a VL domain comprising or consisting of the amino acid sequence of SEQ ID NO: 131 or an amino acid sequence having at least 80%, 90%, 95%, 98% 99% identity thereto.

In certain embodiments, the anti-IgE antibodies are selected from antibodies comprising a variable heavy chain domain (VH) and a variable light chain domain (VL) selected from the following:
(i) a VH domain comprising or consisting of the amino acid sequence of SEQ ID NO: 137 or an amino acid sequence having at least 80%, 90%, 95%, 98% 99% identity thereto, and a VL domain comprising or consisting of the amino acid sequence of SEQ ID NO: 107 or an amino acid sequence having at least 80%, 90%, 95%, 98% 99% identity thereto;
(ii) a VH domain comprising or consisting of the amino acid sequence of SEQ ID NO: 106 or an amino acid sequence having at least 80%, 90%, 95%, 98% 99% identity thereto, and a VL domain comprising or consisting of the amino acid sequence of SEQ ID NO: 138 or an amino acid sequence having at least 80%, 90%, 95%, 98% 99% identity thereto;
(iii) a VH domain comprising or consisting of the amino acid sequence of SEQ ID NO: 137 or an amino acid sequence having at least 80%, 90%, 95%, 98% 99% identity thereto, and a VL domain comprising or consisting of the amino acid sequence of SEQ ID NO: 138 or an amino acid sequence having at least 80%, 90%, 95%, 98% 99% identity thereto;
(iv) a VH domain comprising or consisting of the amino acid sequence of SEQ ID NO: 139 or an amino acid sequence having at least 80%, 90%, 95%, 98% 99% identity thereto, and a VL domain comprising or consisting of the amino acid sequence of SEQ ID NO: 103 or an amino acid sequence having at least 80%, 90%, 95%, 98% 99% identity thereto;
(v) a VH domain comprising or consisting of the amino acid sequence of SEQ ID NO: 102 or an amino acid sequence having at least 80%, 90%, 95%, 98% 99% identity thereto, and a VL domain comprising or consisting of the amino acid sequence of SEQ ID NO: 140 or an amino acid sequence having at least 80%, 90%, 95%, 98% 99% identity thereto;
(vi) a VH domain comprising or consisting of the amino acid sequence of SEQ ID NO: 139 or an amino acid sequence having at least 80%, 90%, 95%, 98% 99% identity thereto, and a VL domain comprising or consisting of the amino acid sequence of SEQ ID NO: 140 or an amino acid sequence having at least 80%, 90%, 95%, 98% 99% identity thereto;
(vii) a VH domain comprising or consisting of the amino acid sequence of SEQ ID NO: 141 or an amino acid sequence having at least 80%, 90%, 95%, 98% 99% identity thereto, and a VL domain comprising or consisting of the amino acid sequence of SEQ ID NO: 99 or an amino acid sequence having at least 80%, 90%, 95%, 98% 99% identity thereto; and
(viii) a VH domain comprising or consisting of the amino acid sequence of SEQ ID NO: 98 or an amino acid sequence having at least 80%, 90%, 95%, 98% 99% identity thereto, and a VL domain comprising or consisting of the amino acid sequence of SEQ ID NO: 142 or an amino acid sequence having at least 80%, 90%, 95%, 98% 99% identity thereto.

In preferred embodiments, the anti-IgE antibodies comprise or consist of a variable heavy chain domain (VH) comprising or consisting of the amino acid sequence of SEQ ID NO: 137 or an amino acid sequence having at least 80%, 90%, 95%, 98% 99% identity thereto, and a variable light chain domain (VL) comprising or consisting of the amino acid sequence of SEQ ID NO: 138 or an amino acid sequence having at least 80%, 90%, 95%, 98% 99% identity thereto.

In preferred embodiments, the anti-IgE antibodies comprise or consist of a variable heavy chain domain (VH) comprising or consisting of the amino acid sequence of SEQ ID NO: 173 or an amino acid sequence having at least 80%, 90%, 95%, 98% 99% identity thereto, and a variable light chain domain (VL) comprising or consisting of the amino acid sequence of SEQ ID NO: 174 or an amino acid sequence having at least 80%, 90%, 95%, 98% 99% identity thereto.

In preferred embodiments, the anti-IgE antibodies comprise or consist of a variable heavy chain domain (VH) comprising or consisting of the amino acid sequence of SEQ ID NO: 173 and a variable light chain domain (VL) comprising or consisting of the amino acid sequence of SEQ ID NO: 174.

In preferred embodiments, the anti-IgE antibodies comprise or consist of a variable heavy chain domain (VH) comprising or consisting of the amino acid sequence of SEQ ID NO: 106 or an amino acid sequence having at least 80%, 90%, 95%, 98% 99% identity thereto, and a variable light chain domain (VL) comprising or consisting of the amino acid sequence of SEQ ID NO: 138 or an amino acid sequence having at least 80%, 90%, 95%, 98% 99% identity thereto.

In preferred embodiments, the anti-IgE antibodies comprise or consist of a variable heavy chain domain (VH) comprising or consisting of the amino acid sequence of SEQ ID NO: 215 or an amino acid sequence having at least 80%, 90%, 95%, 98% 99% identity thereto, and a variable light chain domain (VL) comprising or consisting of the amino acid sequence of SEQ ID NO: 174 or an amino acid sequence having at least 80%, 90%, 95%, 98% 99% identity thereto.

In preferred embodiments, the anti-IgE antibodies comprise or consist of a variable heavy chain domain (VH) comprising or consisting of the amino acid sequence of SEQ ID NO: 215 or an amino acid sequence having at least 80%, 90%, 95%, 98% 99% identity thereto and a variable light chain domain (VL) comprising or consisting of the amino acid sequence of SEQ ID NO: 174.

For embodiments wherein the domains of the antibodies or antigen binding fragments are defined by a particular percentage sequence identity to a reference sequence, the VH and/or VL domains may retain identical CDR sequences to those present in the reference sequence such that the variation is present only within the framework regions.

The exemplary camelid-derived anti-IgE antibodies having any of the specific CDR, VH and/or VL domains recited above may comprise any of the variant Fc domains or FcRn binding fragments thereof according to the embodiments described in sections (i) and (ii) above. The exemplary camelid-derived anti-IgE antibodies having any of the specific CDR, VH and/or VL domains recited above may comprise any of the variant Fc regions or FcRn binding fragments thereof according to the embodiments described in sections (i) and (ii) above.

In certain embodiments, the exemplary camelid-derived anti-IgE antibodies described herein comprise a variant IgG Fc domain or FcRn binding fragment thereof, preferably a variant IgG1 domain or FcRn binding fragment thereof. In certain embodiments, the exemplary camelid-derived anti-IgE antibodies described herein comprise a variant human IgG Fc domain or FcRn binding fragment thereof, preferably a variant human IgG1 Fc domain or FcRn binding fragment thereof.

In certain embodiments, the exemplary camelid-derived anti-IgE antibodies described herein comprise a variant human IgG Fc domain or FcRn binding fragment thereof comprising the amino acids Y, T, E, K, F and Y at EU positions 252, 254, 256, 433, 434 and 436, respectively. In certain embodiments, the exemplary camelid-derived anti-IgE antibodies described herein comprise a variant human IgG1 Fc domain or FcRn binding fragment thereof comprising the amino acids Y, T, E, K, F and Y at EU positions 252, 254, 256, 433, 434 and 436, respectively. In certain embodiments, the exemplary camelid-derived anti-IgE antibodies described herein comprise a variant human IgG Fc region comprising or consisting of two identical variant human IgG Fc domains, wherein each variant Fc domain comprises the amino acids Y, T, E, K, F and Y at EU positions 252, 254, 256, 433, 434 and 436, respectively. In certain embodiments, the exemplary camelid-derived anti-IgE antibodies described herein comprise a variant human IgG1 Fc region comprising or consisting of two identical variant human IgG1 Fc domains, wherein each variant Fc domain comprises the amino acids Y, T, E, K, F and Y at EU positions 252, 254, 256, 433, 434 and 436, respectively.

In certain embodiments, the exemplary camelid-derived anti-IgE antibodies described herein comprise a variant Fc domain comprising or consisting of the amino acid sequence set forth in any one of SEQ ID NOs: 1, 2 or 3. In certain embodiments, the exemplary camelid-derived anti-IgE antibodies described herein comprise a variant Fc region consisting of two variant Fc domains wherein each variant Fc domain comprises or consists of the amino acid sequence set forth in any one of SEQ ID NOs: 1, 2 or 3. In certain embodiments, the exemplary camelid-derived anti-IgE antibodies described herein comprise a variant Fc domain comprising or consisting of the amino acid sequence set forth in any one of SEQ ID NOs: 5, 6 or 7. In certain embodiments, the exemplary camelid-derived anti-IgE antibodies described herein comprise a variant Fc region consisting of two variant Fc domains wherein each variant Fc domain comprises or consists of the amino acid sequence set forth in any one of SEQ ID NOs: 5, 6 or 7. In certain embodiments, the exemplary camelid-derived anti-IgE antibodies described herein comprise a heavy chain constant region comprising or consisting of the amino acid sequence set forth in SEQ ID NO: 4. In certain embodiments, the exemplary camelid-derived anti-IgE antibodies described herein comprise a heavy chain constant region comprising or consisting of the amino acid sequence set forth in SEQ ID NO: 8.

The exemplary camelid-derived anti-IgE antibodies described herein may exhibit pH-dependent antigen binding. In certain embodiments, the anti-IgE antibodies may be engineered so as to exhibit pH-dependent antigen binding by the introduction of one or more substitutions into the variable domains. In preferred embodiments, the anti-IgE antibodies are engineered so as to exhibit pH-dependent antigen binding by introducing one or more substitutions into one or more CDRs of the antibody. The substitutions may introduce one or more His residues into one or more sites of the variable domains, preferably the heavy chain and/or light chain CDRs so as to confer pH-dependent antigen binding. The six heavy chain and light chain CDRs combined may consist of a total of 1-10 His substitutions, optionally 1-5 His substitutions, optionally 1, 2, 3, 4, 5, 6, 7, 8, 9 or 10 His substitutions. The anti-IgE antibodies may be engineered in accordance with the methods described in WO2018/206748. Non-histidine substitutions may also be incorporated into variable domains, particularly the CDRs, of the pH-dependent antibodies described herein.

(vii) Exemplary Anti-IgE Antibodies

As described elsewhere herein, antibodies that bind to IgE are known in the art. The anti-IgE antibodies of the present invention may comprise the CDR, VH and/or VL domain amino acid sequences of any anti-IgE antibody known to exhibit binding specificity for IgE, preferably human IgE.

Exemplary antibodies known to bind IgE include but are not limited to omalizumab and ligelizumab. The anti-IgE antibodies of the invention may comprise CDR, VH and/or VL amino acid sequences derived from omalizumab or ligelizumab.

Therefore, in certain embodiments, the anti-IgE antibodies are selected from antibodies comprising a combination of variable heavy chain CDR3 (HCDR3), variable heavy chain CDR2 (HCDR2) and variable heavy chain CDR1 (HCDR1), variable light chain CDR3 (LCDR3), variable light chain CDR2 (LCDR2) and variable light chain CDR1 (LCDR1) selected from the following:
(i) HCDR3 comprising SEQ ID NO: 145; HCDR2 comprising SEQ ID NO: 144; HCDR1 comprising SEQ ID NO: 143; LCDR3 comprising SEQ ID NO: 149; LCDR2 comprising SEQ ID NO: 148; and LCDR1 comprising SEQ ID NO: 147; and
(ii) HCDR3 comprising SEQ ID NO: 153; HCDR2 comprising SEQ ID NO: 152; HCDR1 comprising SEQ ID NO: 151; LCDR3 comprising SEQ ID NO: 157; LCDR2 comprising SEQ ID NO: 156; and LCDR1 comprising SEQ ID NO: 155;

In certain embodiments, the anti-IgE antibodies are selected from antibodies comprising a variable heavy chain domain (VH) and a variable light chain domain (VL) selected from the following:
(i) a VH domain comprising or consisting of the amino acid sequence of SEQ ID NO: 146 or an amino acid sequence having at least 80%, 90%, 95%, 98% 99% identity thereto, and a VL domain comprising or consisting of the amino acid sequence of SEQ ID NO: 150 or an amino acid sequence having at least 80%, 90%, 95%, 98% 99% identity thereto; and
(ii) a VH domain comprising or consisting of the amino acid sequence of SEQ ID NO: 154 or an amino acid sequence having at least 80%, 90%, 95%, 98% 99% identity thereto, and a VL domain comprising or consisting of the amino acid sequence of SEQ ID NO: 158 or an amino acid sequence having at least 80%, 90%, 95%, 98% 99% identity thereto.

For embodiments wherein the domains of the antibodies or antigen binding fragments are defined by a particular percentage sequence identity to a reference sequence, the VH and/or VL domains may retain identical CDR sequences to those present in the reference sequence such that the variation is present only within the framework regions.

In certain embodiments, the anti-IgE antibodies comprise a variable heavy chain domain (VH) comprising or consisting of the amino acid sequence of SEQ ID NO: 146 and a VL domain comprising or consisting of the amino acid sequence of SEQ ID NO: 150.

In certain embodiments, the anti-IgE antibodies comprise a variable heavy chain domain (VH) comprising or consisting of the amino acid sequence of SEQ ID NO: 154 and a VL domain comprising or consisting of the amino acid sequence of SEQ ID NO: 158.

The anti-IgE antibodies having the CDR, VH and/or VL amino acid sequences recited above may be engineered so as to be pH-dependent, as described in section (iii) above. The exemplary anti-IgE antibodies described herein may be engineered so as to exhibit pH-dependent antigen binding by the introduction of one or more substitutions into the variable domains. In preferred embodiments, the anti-IgE antibodies are engineered so as to exhibit pH-dependent antigen binding by introducing one or more substitutions into one or more CDRs of the antibody. The substitutions may introduce one or more His residues into one or more sites of the variable domains, preferably the heavy chain and/or light chain CDRs so as to confer pH-dependent antigen binding. The six heavy chain and light chain CDRs combined may consist of a total of 1-10 His substitutions, optionally 1-5 His substitutions, optionally 1, 2, 3, 4, 5, 6, 7, 8, 9 or 10 His substitutions. The anti-IgE antibodies may be engineered in accordance with the methods described in WO2018/206748. Non-histidine substitutions may also be incorporated into variable domains, particularly the CDRs, of the pH-dependent antibodies described herein.

Exemplary pH-dependent anti-IgE antibodies in accordance with the invention are described below with reference to specific CDR, VH and/or VL sequences.

In certain embodiments, pH-dependent anti-IgE antibodies of the invention comprise:
a variable heavy chain CDR3 comprising or consisting of SEQ ID NO: 197 [ATHYFGHWHFAV];
a variable heavy chain CDR2 comprising or consisting of SEQ ID NO: 198 [SIHYDHSTNYNPSVKG];
a variable heavy chain CDR1 comprising or consisting of SEQ ID NO: 195 [SGHRWE];
a variable light chain CDR3 comprising or consisting of SEQ ID NO: 201 [QQNAEDPYT];
a variable light chain CDR2 comprising or consisting of SEQ ID NO: 200 [WGSYLRS]; and
a variable light chain CDR1 comprising or consisting of SEQ ID NO: 203 [RASQSVDYDGDHYMN].

In certain embodiments, pH-dependent anti-IgE antibodies of the invention comprise:
a variable heavy chain CDR3 comprising or consisting of SEQ ID NO: 199 [ATHYFGHHHFAV];
a variable heavy chain CDR2 comprising or consisting of SEQ ID NO: 196 [SIHYDGSTNYNPSVKG];
a variable heavy chain CDR1 comprising or consisting of SEQ ID NO: 195 [SGHRWE];
a variable light chain CDR3 comprising or consisting of SEQ ID NO: 201 [QQNAEDPYT];
a variable light chain CDR2 comprising or consisting of SEQ ID NO: 200 [WGSYLRS]; and
a variable light chain CDR1 comprising or consisting of SEQ ID NO: 147 [RASQSVDYDGDSYMN].

In certain embodiments, pH-dependent anti-IgE antibodies of the invention comprise:
a variable heavy chain CDR3 comprising or consisting of SEQ ID NO: 180 [FSHFSGSNHDYFDY];
a variable heavy chain CDR2 comprising or consisting of SEQ ID NO: 152 [EIDPGTFTTNYNEKFKA];
a variable heavy chain CDR1 comprising or consisting of SEQ ID NO: 179 [WYHLE];
a variable light chain CDR3 comprising or consisting of SEQ ID NO: 157 [QQSWSWPTT];
a variable light chain CDR2 comprising or consisting of SEQ ID NO: 156 [YASESIS]; and
a variable light chain CDR1 comprising or consisting of SEQ ID NO: 155 [RASQSIGTNIH].

In certain embodiments, the anti-IgE antibodies comprise or consist of a variable heavy chain domain (VH) comprising or consisting of the amino acid sequence of SEQ ID NO: 206 or an amino acid sequence having at least 80%, 90%, 95%, 98% 99% identity thereto, and a variable light chain domain (VL) comprising or consisting of the amino acid sequence of SEQ ID NO: 211 or an amino acid sequence having at least 80%, 90%, 95%, 98% 99% identity thereto.

In preferred embodiment, the anti-IgE antibodies comprise or consist of a variable heavy chain domain (VH) comprising or consisting of the amino acid sequence of SEQ ID NO: 206, and a variable light chain domain (VL) comprising or consisting of the amino acid sequence of SEQ ID NO: 211.

In certain embodiments, the anti-IgE antibodies comprise or consist of a variable heavy chain domain (VH) comprising or consisting of the amino acid sequence of SEQ ID NO: 207 or an amino acid sequence having at least 80%, 90%, 95%, 98% 99% identity thereto, and a variable light chain domain (VL) comprising or consisting of the amino acid sequence of SEQ ID NO: 209 or an amino acid sequence having at least 80%, 90%, 95%, 98% 99% identity thereto.

In preferred embodiment, the anti-IgE antibodies comprise or consist of a variable heavy chain domain (VH) comprising or consisting of the amino acid sequence of SEQ ID NO: 207, and a variable light chain domain (VL) comprising or consisting of the amino acid sequence of SEQ ID NO: 209.

In certain embodiments, the anti-IgE antibodies comprise or consist of a variable heavy chain domain (VH) comprising or consisting of the amino acid sequence of SEQ ID NO: 186 or an amino acid sequence having at least 80%, 90%, 95%, 98% 99% identity thereto, and a variable light chain domain (VL) comprising or consisting of the amino acid sequence of SEQ ID NO: 158 or an amino acid sequence having at least 80%, 90%, 95%, 98% 99% identity thereto.

In preferred embodiment, the anti-IgE antibodies comprise or consist of a variable heavy chain domain (VH) comprising or consisting of the amino acid sequence of SEQ ID NO: 186, and a variable light chain domain (VL) comprising or consisting of the amino acid sequence of SEQ ID NO: 158.

The exemplary anti-IgE antibodies having any of the specific CDR, VH and/or VL domains recited above may comprise any of the variant Fc domains or FcRn binding fragments thereof according to the embodiments described in sections (i) and (ii) above. The exemplary anti-IgE antibodies having any of the specific CDR, VH and/or VL domains recited above may comprise any of the variant Fc regions or FcRn binding fragments thereof according to the embodiments described in sections (i) and (ii) above.

In certain embodiments, the exemplary anti-IgE antibodies described herein comprise a variant IgG Fc domain or FcRn binding fragment thereof, preferably a variant IgG1 domain or FcRn binding fragment thereof. In certain embodiments, the exemplary anti-IgE antibodies described herein comprise a variant human IgG Fc domain or FcRn binding fragment thereof, preferably a variant human IgG1 domain or FcRn binding fragment thereof.

In certain embodiments, the exemplary anti-IgE antibodies described herein comprise a variant human IgG Fc domain or FcRn binding fragment thereof comprising the amino acids Y, T, E, K, F and Y at EU positions 252, 254, 256, 433, 434 and 436, respectively. In certain embodiments, the exemplary anti-IgE antibodies described herein comprise a variant human IgG1 Fc domain or FcRn binding fragment thereof comprising the amino acids Y, T, E, K, F and Y at EU positions 252, 254, 256, 433, 434 and 436, respectively. In certain embodiments, the exemplary anti-IgE antibodies described herein comprise a variant human IgG Fc region comprising or consisting of two identical variant human IgG Fc domains, wherein each variant Fc domain comprises the amino acids Y, T, E, K, F and Y at EU positions 252, 254, 256, 433, 434 and 436, respectively. In certain embodiments, the exemplary anti-IgE antibodies described herein comprise a variant human IgG1 Fc region comprising or consisting of two identical variant human IgG1 Fc domains, wherein each variant Fc domain comprises the amino acids Y, T, E, K, F and Y at EU positions 252, 254, 256, 433, 434 and 436, respectively.

In certain embodiments, the exemplary anti-IgE antibodies described herein comprise a variant Fc domain comprising or consisting of the amino acid sequence set forth in any one of SEQ ID NOs: 1, 2 or 3. In certain embodiments, the exemplary anti-IgE antibodies described herein comprise a variant Fc region consisting of two variant Fc domains wherein each variant Fc domain comprises or consists of the amino acid sequence set forth in any one of SEQ ID NOs: 1, 2 or 3. In certain embodiments, the exemplary anti-IgE antibodies described herein comprise a variant Fc domain comprising or consisting of the amino acid sequence set forth in any one of SEQ ID NOs: 5, 6 or 7. In certain embodiments, the exemplary anti-IgE antibodies described herein comprise a variant Fc region consisting of two variant Fc domains wherein each variant Fc domain comprises or consists of the amino acid sequence set forth in any one of SEQ ID NOs: 5, 6 or 7. In certain embodiments, the exemplary anti-IgE antibodies described herein comprise a heavy chain constant region comprising or consisting of the amino acid sequence set forth in SEQ ID NO: 4. In certain embodiments, the exemplary anti-IgE antibodies described herein comprise a heavy chain constant region comprising or consisting of the amino acid sequence set forth in SEQ ID NO: 8.

### B. Polynucleotides Encoding Anti-IgE Antibodies

The invention also provides polynucleotide molecules encoding the anti-IgE antibodies of the invention or fragments thereof, also expression vectors containing said nucleotide sequences of the invention operably linked to regulatory sequences which permit expression of the antibodies or fragments thereof in a host cell or cell-free expression system, and a host cell or cell-free expression system containing this expression vector.

Polynucleotide molecules encoding the antibodies of the invention include, for example, recombinant DNA molecules. The terms “nucleic acid”, “polynucleotide” or a “polynucleotide molecule” as used herein interchangeably and refer to any DNA or RNA molecule, either single- or double-stranded and, if single-stranded, the molecule of its complementary sequence. In discussing nucleic acid molecules, a sequence or structure of a particular nucleic acid molecule may be described herein according to the normal convention of providing the sequence in the 5′ to 3′ direction. In some embodiments of the invention, nucleic acids or polynucleotides are “isolated.” This term, when applied to a nucleic acid molecule, refers to a nucleic acid molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or non-human host organism. When applied to RNA, the term “isolated polynucleotide” refers primarily to an RNA molecule encoded by an isolated DNA molecule as defined above. Alternatively, the term may refer to an RNA molecule that has been purified/separated from other nucleic acids with which it would be associated in its natural state (i.e., in cells or tissues). An isolated polynucleotide (either DNA or RNA) may further represent a molecule produced directly by biological or synthetic means and separated from other components present during its production.

For recombinant production of an antibody according to the invention, a recombinant polynucleotide encoding it may be prepared (using standard molecular biology techniques) and inserted into a replicable vector for expression in a chosen host cell, or a cell-free expression system. Suitable host cells may be prokaryote, yeast, or higher eukaryote cells, specifically mammalian cells. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen. Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); mouse myeloma cells SP2/0-AG14 (ATCC CRL 1581; ATCC CRL 8287) or NS0 (HPA culture collections no. 85110503); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2), as well as DSM's PERC-6 cell line. Expression vectors suitable for use in each of these host cells are also generally known in the art.

It should be noted that the term “host cell” generally refers to a cultured cell line. Whole human beings into which an expression vector encoding an antibody according to the invention has been introduced are explicitly excluded from the definition of a “host cell”.

### C. Antibody Production

In a further aspect, the invention also provides a method of producing anti-IgE antibodies of the invention which comprises culturing a host cell (or cell free expression system) containing polynucleotide (e.g. an expression vector) encoding the antibody under conditions which permit expression of the antibody, and recovering the expressed antibody. This recombinant expression process can be used for large scale production of anti-IgE antibodies according to the invention, including monoclonal antibodies intended for human therapeutic use. Suitable vectors, cell lines and production processes for large scale manufacture of recombinant antibodies suitable for in vivo therapeutic use are generally available in the art and will be well known to the skilled person.

### D. Pharmaceutical Compositions

The scope of the invention includes pharmaceutical compositions, containing one or a combination of anti-IgE antibodies of the invention formulated with one or more pharmaceutically acceptable carriers or excipients. Such compositions may include one or a combination of (e.g., two or more different) anti-IgE antibodies. Techniques for formulating monoclonal antibodies for human therapeutic use are well known in the art and are reviewed, for example, in Wang et al., Journal of Pharmaceutical Sciences, Vol. 96, pp 1-26, 2007, the contents of which are incorporated herein in their entirety.

Pharmaceutically acceptable excipients that may be used to formulate the compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

In certain embodiments, the pharmaceutical compositions are formulated for administration to a subject via any suitable route of administration including but not limited to intramuscular, intravenous, intradermal, intraperitoneal injection, subcutaneous, epidural, nasal, oral, rectal, topical, inhalational, buccal (e.g., sublingual), and transdermal administration. In preferred embodiments, the composition is formulated for intravenous or subcutaneous administration.

### E. Methods of Treatment

The anti-IgE antibodies and pharmaceutical compositions as described herein are intended for use in methods of treatment. The present invention thus provides anti-IgE antibodies in accordance with the first aspect of the invention or pharmaceutical compositions comprising the same for use as medicaments.

Further provided are methods of treating an antibody-mediated disorder in a subject, the methods comprising administering to a patient in need thereof a therapeutically effective amount of an anti-IgE antibody in accordance with the first aspect of the invention or a pharmaceutical composition comprising the same. The invention also provides anti-IgE antibodies in accordance with the first aspect of the invention or pharmaceutical compositions comprising the same for use in the treatment of an antibody-mediated disorder in a subject in need thereof. The subject is preferably human. All embodiments described above in relation to the anti-IgE antibodies and pharmaceutical compositions of the invention are equally applicable to the methods described herein.

In certain embodiments, the antibody-mediated disorder treated in accordance with the methods described herein is an IgE-mediated disorder. In certain embodiments, the antibody-mediated disorder is an autoimmune disorder. Autoimmune disorders or diseases that may be treated in accordance with the methods described herein include but are not limited to allogenic islet graft rejection, alopecia areata, amyloidosis, ankylosing spondylitis, antiphospholipid syndrome, autoimmune Addison's disease, Alzheimer's disease, antineutrophil cytoplasmic autoantibodies (ANCA), autoimmunocytopenia, autoimmune diseases of the adrenal gland, autoimmune hemolytic anemia, autoimmune hepatitis, autoimmune myocarditis, autoimmune neutropenia, autoimmune oophoritis and orchitis, autoimmune thrombocytopenia, autoimmune urticaria, Behcet's disease, bullous pemphigoid, cardiomyopathy, Castleman's syndrome, celiac spruce-dermatitis, chronic fatigue immune disfunction syndrome, chronic inflammatory demyelinating polyneuropathy (CIDP), chronic inducible urticaria, chronic spontaneous urticaria, Churg-Strauss syndrome, cicatrical pemphigoid, CREST syndrome, cold agglutinin disease, Crohn's disease, dermatomyositis, discoid lupus, essential mixed cryoglobulinemia, factor VIII deficiency, fibromyalgia-fibromyositis, glomerulonephritis, Grave's disease, Guillain-Barre Syndrome, Goodpasture's syndrome, graft-versus-host disease (GVHD), Hashimoto's thyroiditis, hemophilia A, idiopathic pulmonary fibrosis, idiopathic thrombocytopenia purpura (ITP), IgA neuropathy, IgM polyneuropathies, immune mediated thrombocytopenia, juvenile arthritis, Kawasaki's disease, lichen plantus, systemic lupus erythematosis, lupus nephritis, Meniere's disease, mixed connective tissue disease, mycosis fungoides, multiple sclerosis, type 1 diabetes mellitus, Multifocal motor neuropathy (MMN), myasthenia gravis, bullous pemphigoid, pemphigus vulgaris, pemphigus foliaceus, pernicious anemia, polyarteritis nodosa, polychrondritis, polyglandular syndromes, polymyalgia rheumatica, polymyositis and dermatomyositis, polyneuritis, primary agammaglobinulinemia, primary biliary cirrhosis, psoriasis, psoriatic arthritis, Reynauld's phenomenon, Reiter's syndrome, rheumatoid arthritis, sarcoidosis, scleroderma, Sharp syndrome, Sjorgen's syndrome, solid organ transplant rejection, stiff-man syndrome, systemic lupus erythematosus, takayasu arteritis, toxic epidermal necrolysis (TEN), Stevens Johnson syndrome (SJS), temporal arteristis/giant cell arteritis, thrombotic thrombocytopenia purpura, thrombocytopenia purpura, ulcerative colitis, uveitis, dermatitis herpetiformis vasculitis, anti-neutrophil cytoplasmic antibody-associated vasculitides, vitiligo, and Wegener's granulomatosis.

In preferred embodiments, the methods described herein are for the treatment of chronic spontaneous urticaria or bullous pemphigoid. As explained elsewhere herein, these disorders are characterised by the presence of both autoreactive IgE antibodies and/or autoreactive IgG antibodies. The anti-IgE antibodies described herein are thus particularly suited to the treatment of these two autoimmune disorders since the anti-IgE antibodies of the invention can target both forms of autoreactive antibody thereby depleting both IgE and IgG autoantibody levels in the CSU or BP patient. Additional indications involving both IgE and/or IgG autoantibodies include systemic lupus erythematosus, lupus nephritis, autoimmune uveitis, allergic bronchopulmonary aspergillosis, Churg-Strauss syndrome, Wegener's granulomatosis, and thyroid autoimmune diseases such as Grave's disease and Hashimoto's thyroiditis.

The methods described herein may include administration of further therapeutic agents.

For embodiments wherein the methods are for treating chronic spontaneous urticaria, the methods may comprise the administration of one or more further therapeutic agents selected from anti-histamines, cyclosporine, dapsone, hydroxychloroquine, sulfasalazine, colchicine, methotrexate, IVIG, corticosteroids, H2 receptor antagonists or leukotriene antagonists. For embodiments wherein the methods are for treating bullous pemphigoid, the methods may comprise the administration of one or more further therapeutic agents selected from a corticosteroid, rituximab, or immunosuppressants such as azathioprine, mycophenolate, dapsone, methotrexate, chlorambucil and cyclophosphamide.

Patients or subjects treated in accordance with the methods described herein may already be receiving treatment or may have failed on a previous treatment. For example, patients or subjects treated in accordance with the methods described herein may be receiving or have already received treatments such as corticosteroids, immunosuppressants, IViG, anti-histamines and/or Omalizumab

### INCORPORATION BY REFERENCE

Various publications are cited in the foregoing description and throughout the following examples, each of which is incorporated by reference herein in its entirety.

### EXAMPLES

The invention will be further understood with reference to the following non-limiting examples.

### Example 1. Production of Anti-IgE Antibodies in Llama

### A. Immunization of Llamas

Four llamas were immunized with recombinant human immunoglobulin E (hIgE) (protein L purified IgE from Abcam; cat #ab65866) by intramuscular injection in the neck after mixing with Incomplete Freund's Adjuvant. The immunization scheme is summarized in Table 6.

TABLE 6
Summary of immunization schedule and tissue collection

| | | | Amount of | |
|---|---|---|---|---|
| Week | Date | Day | antigen | Tissue collection |
| 0 | 1 Nov. 2012 | | | Preimmune serum |
| 1 | 1 Nov. 2012 | 1 | 100 μg | |
| 2 | 8 Nov. 2012 | 8 | 100 μg | |
| 3 | 15 Nov. 2012 | 15 | 50 μg | |
| 4 | 22 Nov. 2012 | 22 | 50 μg | |
| 5 | 29 Dec. 2012 | 28 | 50 μg | |
| 6 | 6 Nov. 2012 | 34 | 50 μg | |
| 7 | 10 Nov. 2012 | 38 | | 400 ml immune blood |
| | | | | 10 ml immune blood (plasma) |
| 12(*) | 17 Jan. 2013 | | 50 μg | |
| 13(*) | 24 Jan. 2013 | | 50 μg | |
| 14(*) | 29 Jan. 2013 | | | 400 ml immune blood |
| | | | | immune serum |

(*)Llama Adelio and Shanio were boosted with 2 injections of hIgE.

Four to five days after the last immunization, 400 mL of blood from the immunized llamas was collected to isolate the PBMC and allow RNA extraction. In order to determine the immune response of the immunized llamas, an enzyme-linked immunosorbent assay (ELISA) set-up was used.

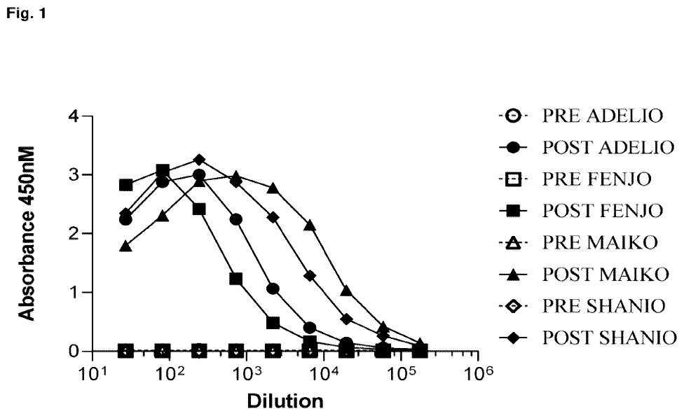

To carry out the ELISA, a Maxisorp plate was coated with 1 μg/ml of hIgE O/N at 4° C. Plates were washed with PBS-Tween and blocked for 2 hours with PBS+1% casein. Serial dilutions of llama serum pre- and post-immunization were added to the wells of the plate and incubated for 1h. Llama Immunoglobulin (Ig) bound to coated hIgE was detected with a mouse anti-llama VH specific antibody (27E10). Detection was realized with an anti-mouse IgG-HRP (DAMPO). Finally, after the addition of TMB, the reaction was stopped with 0.5M H₂SO₄ and absorbance was measured at 450 nm (Tecan Sunrise, Magellan software). All immunized llamas showed a specific immune response against hIgE (see FIG. 1).

### B. Library Construction (Fab)

Fab libraries were constructed as follows: mRNA was purified from PBMCs isolated from the blood of the immunized llamas using the Rneasy Midi kit from Qiagen. RNA integrity was verified via the Experion StdSens Analysis Kit. The mRNA was reverse transcribed with random hexamer primers to obtain cDNA. For construction of heavy and light chain libraries, a two-step PCR was used. First, non-tagged primers were used directly on the cDNA to amplify the VH-CH1, VL-CL and Vk-Ck. The PCR product was then purified and used in a second PCR with tagged primers to amplify the VH-CH, VL and Vk. The light chains (Vλ-Cλ or Vκ-Cκ) were re-cloned in the heavy chain (VH-CH) library derived from the same llama, to form the Fab library.

TABLE 7
Size of the libraries generated (CFU)

| | | | | Fab library | Fab library |
|---|---|---|---|---|---|
| | | | | VH-CH/ | VH-CH/ |
| | Vκ-Cκ | Vλ-Cλ | VH-CH1 | Vκ-Cκ | Vλ-Cλ |
| ADELIO | 3.0 × 10⁹ | 6 × 10⁸ | 1.2 × 10⁸ | 3.6 × 10⁹ | 8.0 × 10⁸ |
| FENJO | 8.8 × 10⁸ | 1.1 × 10⁹ | 1.2 × 10⁹ | 1.6 × 10⁹ | 8.8 × 10⁸ |
| MAIKO | 5.6 × 10⁸ | 1.1 × 10⁹ | 1.2 × 10⁹ | 7.6 × 10⁸ | 1.2 × 10⁹ |
| SHANIO | 2.2 × 10⁸ | 3.0 × 10⁹ | 1.5 × 10⁹ | 3.0 × 10⁹ | 3.2 × 10⁹ |

Enrichment of phages expressing specific hIgE Fab fragments were performed by three rounds of selection on immobilized hIgE. Two different selection methods were used differing only in the type of elution after phage selection.

The initial selection of the appropriate Fab clones specific for hIgE was carried out by a bio-panning approach. Briefly, hIgE was immobilized on Maxisorp ELISA plate, then the Fab phage library (Input), in TBS pH7.4, was added. Unbound phages were removed via multiple washing steps. Finally, the bound phages were eluted with Trypsin or with TBS pH 5.5. E. coli were infected with the eluted material in order to amplify the selected phages. This process resulted in the enrichment of the phage population expressing Fab with high affinity to hIgE. At the end of the round of selection, the number of eluted phages was estimated by titration of infected E. coli, spotted (from 10-1 to 10-6) on Petri dishes containing solid LB medium with ampicillin and glucose. The first round of selection of the Lambda and Kappa library from both llamas resulted in a minor enrichment of specific phages to hIgE. The second and third rounds of selection resulted in an enrichment of phages expressing Fab with probably a higher affinity for hIgE. Two different selection campaigns were performed:
Campaign 1: all third rounds of selection were done with hIgE purchased at Abcam (cat #ab65866). Clone starting with 1-9 were obtained in this campaign.
Campaign 2: first round of selection was done with Abcam hIgE. Second and third rounds of selection were done with hIgE purchased at Kerafast: (cat #EX0011). Clone starting with 10-20 were obtained in this campaign.

Tables 3-5 shows the coating amount used for different rounds of selections. Single clone generation resulted in the creation of Master plates. From these Master plates, periplasmic master plates (PMP) were produced. The antibody fragments in Fab format can be secreted into the periplasmic space of E. coli bacteria by induction with IPTG. For this purpose, single clones from the Master plates were first amplified in 96 well format (deep well), and production of the Fab was induced by an overnight incubation with IPTG. The next day, the bacteria were lysed by two cycles of freeze/thaw (−80° C. and −20° C.). After centrifugation, the supernatant (periplasmic extract) was collected and transferred in a separate 96 well plate in order to test their binding capacity (ELISA and Biacore).

TABLE 8
First round selection (R1)

| | Phage | hIgE coating | |
|---|---|---|---|
| | (μl) | (μg/ml) | elution |
| R1a | 10 | 50 | Trypsin |
| R1b | 10 | 5 | Trypsin |
| R1c | 10 | 50 | pH5.5 |
| R1d | 10 | 5 | pH5.5 |

TABLE 9
Second round of selection (R2)

| | Phage | hIgE coating | |
|---|---|---|---|
| | (μl) | (μg/ml) | elution |
| R2a | 5 | 20 | Trypsin |
| R2b | 5 | 2 | Trypsin |
| R2c | 5 | 20 | pH 5.5 |
| R2d | 5 | 2 | pH 5.5 |

TABLE 10
Third round of selection (R3)

| | Phage | Coating | |
|---|---|---|---|
| | (μl) | (μg/ml) | elution |
| R3a | 1 | 2 | Trypsin |
| R3b | 1 | 0.2 | Trypsin |
| R3c | 1 | 2 | pH 5.5 |
| R3d | 1 | 0.2 | pH 5.5 |

### C. Screening of the Fab Periplasmic Extracts by ELISA

In order to test the binding capacity of the Fab to hIgE, an ELISA binding assay was established. Briefly, a Maxisorp plate was coated with hIgE (1 μg/mL), then blocked with PBS 1% Casein, before being incubated with the periplasmic extract (dilution 1/4 in PBS) containing the Fab-Myc. Detection of the binders was carried out using an anti-Myc-HRP antibody. Absorbance was measured at 450 nm (reference at 620 nm) with Tecan instrument.

### D. Screening of the Fab Periplasmic Extracts by Competition ELISA

To identify Fab blocking the IgE-FcεRIα interaction, a competition ELISA binding assay was established. Briefly, a Maxisorp plate was coated with 1 μg/ml of soluble FcεRIα (R&D system, cat #6678-FC), then blocked with PBS 1% Casein. Biotinylated hIgE was pre-incubated with the periplasmic extract (dilution 1/4 in PBS) before being added to the FcεRIα coated well. hIgE binding was detected using streptavidin-HRP reagent. Absorbance was measured at 450 nm (reference at 620 nm) with Tecan instrument.

E. Screening of the pH-Dependent Binding Fab Periplasmic Extracts by SPR

Binding capacity to hIgE of was analyzed on Biacore T3000 at pH 7.4 and pH 5.5. For this purpose, a CM5 Chip was coated with hIgE at 2000 RU. Periplasmic extract (dilution 1/10 in HBSEP pH7.4 buffer or HBSEP pH5.5) were injected to the Chip coated with hIgE. Raw data were analyzed via BIA evaluation software with a blank subtraction.

The CDR, VH and VL sequences of pH-dependent IgE binding clones are shown in Tables 11-13 below.

TABLE 11
Heavy chain CDR sequences of Fabs binding to IgE

| Fab clone | CDR1 | SEQ ID NO. | CDR2 | SEQ ID NO. | CDR3 | SEQ ID NO. |
|---|---|---|---|---|---|---|
| 3D6 | SYYMT | 9 | SIYSDGSNTYYADSVKG | 10 | DLKARYSGSYHDEGYDY | 11 |
| 16E4 | SYYMS | 12 | SIYSDGSYAYYADSVKG | 13 | DLKARYSGTYHDEGYDY | 14 |
| 3A1 | NYAMS | 15 | AISWNGGSTYYAESMKG | 16 | DLLVAARGGMDY | 17 |
| 3D1 | SYYMS | 12 | SIYSDGRGSKTFYADSVKG | 18 | DLLVAARGSM | 19 |
| 13E4 | SYVMS | 20 | SIYHDGSHTYYADFVKG | 21 | GTSYSGSYYYTDPFFGS | 22 |
| 18B9 | SYVMS | 20 | SIYSDGSHTYYADSVKG | 23 | NLEHYSGSYYYTDPRYDY | 24 |
| 20D5 | SYVMT | 25 | SIYSDGSHTYYADSVKD | 26 | DAEYYSGSYYYTDTKYDY | 27 |
| 18E2 | SYVMS | 20 | SIYHDGSHTYYADFVKG | 21 | GTSYSGSYYYTDPFFGS | 22 |
| 14F10 | DYDMS | 28 | IISWNGGSTDYAESMKG | 29 | HSVGRNGYDY | 30 |
| 15C3 | NYYMS | 31 | SIYSDGGYTYYADSVKG | 32 | DLKPRNSGTYHDEGYDD | 33 |
| 15D12 | TYVMS | 34 | SIYSDGSHTYYADSVKG | 23 | GTSYSGSYYYTDPFFGS | 22 |
| 17A10 | TSYYAWN | 35 | VIAYDGSTDYSPSLKS | 36 | DYRINSDYAGGYDY | 37 |
| 17G12 | SYVMS | 20 | SIYHDGSHTYYADFVKG | 21 | GTSYSASYYYTDPFFGS | 38 |
| 17H2 | SYYMS | 12 | SISSDGSNPYYADSVKG | 39 | DTLTGASYSDSLYDY | 40 |
| 19H2 | SYAMS | 41 | SIYSYSSNTYYADSVKG | 42 | TTLSRLTYSDYRYDY | 43 |
| 20A1 | SYYMS | 12 | SIYSDGSYAYYADSVKG | 13 | DLKARYSGTYHDEGYDY | 14 |
| 20D2 | SYYMS | 12 | SIYSDDSNTDYADSVKG | 44 | ATGTVGYYSDYFYDY | 45 |
| 20G5 | DYAMS | 46 | GISWKGGIIYYAESMEG | 47 | ALGTVASGQYDY | 48 |
| 21A1 | SYYMS | 12 | SISSDGSNTYYADSVKG | 49 | DDNSGSDYEFGYDY | 50 |
| 4D8 | SSYYDWT | 51 | VIHYDGSTYYSPSLKS | 52 | SYSSSPWDYDY | 53 |

TABLE 12
Light chain CDR sequences of Fabs binding to IgE

| Fab clone | CDR1 | SEQ ID NO. | CDR2 | SEQ ID NO. | CDR3 | SEQ ID NO. |
|---|---|---|---|---|---|---|
| 3D6 | QGGSLGSSYAH | 54 | DDDSRPS | 55 | QSADSSGNPV | 56 |
| 16E4 | QGGSLGATYAY | 57 | DDDSRPS | 55 | QSAYSNGNAV | 58 |
| 3A1 | QGGTLGSYGAH | 59 | GDNSRPS | 60 | QSFDYSGNAV | 61 |
| 3D1 | QGGTLGSYGAH | 59 | GDNSRPS | 60 | QSFDYSGNAV | 61 |
| 13E4 | QGGSLGSNYAY | 62 | DDDSRPS | 55 | QSADSNGNAV | 63 |
| 18B9 | QGGSLGSSYVH | 64 | DGDSRPS | 65 | QSADSSGNAV | 66 |
| 20D5 | QGGSLGSSYAH | 54 | ADDSRPS | 67 | QSADSSGNAV | 66 |
| 18E2 | QGDRLGSRYIY | 68 | DDDRRPS | 69 | QSADSSGNPV | 56 |
| 14F10 | QGGSLGTSYAY | 70 | DDDNRPS | 71 | QSEDTSSNFV | 72 |
| 15C3 | QGGSLGSSYAH | 54 | DDDSRPS | 55 | QSADSSGNPV | 56 |
| 15D12 | QGGSLGSNYAY | 62 | DDDSRPS | 55 | QSADSNGNAV | 63 |
| 17A10 | TGSSSNIGGGYYLS | 73 | NANNRAS | 74 | GCYDSSLSTPV | 75 |
| 17G12 | QGGSLGSNYAY | 62 | DDDSRPS | 55 | QSADSNGNAV | 63 |
| 17H2 | QGGSLGGSYAH | 76 | DDTSRPS | 77 | QSSYSSGNPV | 78 |
| 19H2 | QGDNLGNNYVQ | 79 | DDNRRPS | 80 | QASDSSGNAV | 81 |
| 20A1 | QGGNLGSSYAH | 82 | DDDSRPS | 55 | QSADSSGNPV | 56 |
| 20D2 | QGGSLGSSYAH | 54 | DDDSRPS | 55 | QSADSSGNAV | 66 |
| 20G5 | AGTSNDVGYGNYVS | 83 | DVNKRAS | 84 | ASYRTNNNVV | 85 |
| 21A1 | QGDNFGSYYAS | 86 | KDSERPS | 87 | LSYDNNGAPV | 88 |
| 4D8 | AGTSSDIGGYNSVS | 89 | EVNKRAS | 90 | ASYRNSNNVV | 91 |

TABLE 13
VH and VL sequences of Fabs binding to IgE

| | | SEQ ID | | SEQ ID |
|---|---|---|---|---|
| Fab clone | VH | NO. | VL | NO. |
| 3D6 | QVQLVESGGGLVQPGGSLRLSCAASGFTFSS | 92 | QSALTQPSALSVTLGQTAKITCQGGSLGSSYAHWY | 93 |
| | YYMTWVRQAPGKGLEWVSSIYSDGSNTYYA | | QQKPGQAPVLVIYDDDSRPSGIPERFSGSSSGGRA | |
| | DSVKGRFTISRDNAKNTLHLQMNSLKSEDTA | | TLTISGAQAEDEGDYYCQSADSSGNPVFGGGTKLT | |
| | VYYCAKDLKARYSGSYHDEGYDYWGQGTQV | | VL | |
| | TVSS | | | |
| 16E4 | EVQLVESGGGLVQPGGSLRLSCAASGFTFSS | 94 | SSALTQPSALSVTLGQSAKITCQGGSLGATYAYWY | 95 |
| | YYMSWVRQAPGKGLEWVSSIYSDGSYAYYA | | QQKPGQAPVLVIYDDDSRPSGIPERFSGSSSGGRA | |
| | DSVKGRFTISRDNAKNTLYLQMNSLKSEDTAV | | TLTISGAQAEDEGDYYCQSAYSNGNAVFGGGTHLT | |
| | YYCAKDLKARYSGTYHDEGYDYWGQGTQVT | | VL | |
| | VSS | | | |
| 3A1 | EVQVQESGGGLVQPGGSLRLSCAASGFTFD | 96 | SSALTQPSAVSVSLEQTARITCQGGTLGSYGAHWY | 97 |
| | NYAMSWVRQAPGKGLEWVSAISWNGGSTYY | | QQKPGQAPVLLIYGDNSRPSGIPERFSGTRSGGTA | |
| | AESMKGRFTISRDNAKNMLYLQMNSLKSEDT | | TLTISGAQAEDEADYYCQSFDYSGNAVFGGGTHLT | |
| | AVYYCAKDLLVAARGGMDYWGKGTLVTVSS | | VL | |
| 3D1 | QLQVVESGGGLVQPGGSLRLSCAASGFTFSS | 98 | NFMLTQPSAVSVSLEQTARITCQGGTLGSYGAHWY | 99 |
| | YYMSWVRQAPGKGLEWVSSIYSDGRGSKTF | | QQKPGQAPVLLIYGDNSRPSGIPERFSGTRSGGTA | |
| | YADSVKGRFTISRDNAKNTLYLQMNSLKSEDT | | TLTISGAQAEDEADYYCQSFDYSGNAVFGGGTHLT | |
| | AVYFCAKDLLVAARGSMDYWGQGTQVTVSS | | VL | |
| 13E4 | ELQLVESGGGLVQPGGSLRLSCAASGFTFSS | 100 | HSAVTQPSALSVTLGQTAKITCQGGSLGSNYAYWY | 101 |
| | YVMSWVRQAPGKGLEWVSSIYHDGSHTYYA | | QQKPGQAPVLVIYDDDSRPSGIPERFSGSSSGGTA | |
| | DFVKGRFTISRDNAKNTLYLQMNSLKSEDTAV | | TLTISGAQAEDEGDYYCQSADSNGNAVFGGGTHLT | |
| | YYCASGTSYSGSYYYTDPFFGSWGQGTQVT | | VL | |
| | VSS | | | |
| 18B9 | QVQLVESGGGLVQPGGSLRLSCAASGFTFSS | 102 | SSALTQPSALSVTLGQTAKITCQGGSLGSSYVHWY | 103 |
| | YVMSWVRQAPGKGLEWVSSIYSDGSHTYYA | | QQKPGQAPVLVIYDGDSRPSGIPERFSGSSSGGTA | |
| | DSVKGRFTISRDNAKNTLYLQMNSLKSEDTAV | | TLTISGAQAEDEDDYYCQSADSSGNAVFGGGTHLT | |
| | YYCAKNLEHYSGSYYYTDPRYDYWGQGTQV | | VL | |
| | TVSS | | | |
| 20D5 | QLQLVESGGGLVQPGGSLRLSCAASGFAFSS | 104 | SSALTQPSALSVTLGQTAKITCQGGSLGSSYAHWY | 105 |
| | YVMTWVRQAPGKGLEWVSSIYSDGSHTYYA | | QQKPGQAPVLVIYADDSRPSGIPERFSGSSSGGTA | |
| | DSVKDRFTISRDNAKNTLFLQMNSLKSEDTAV | | TLTISGAQAEDEGDYYCQSADSSGNAVFGGGTHLT | |
| | YYCAKDAEYYSGSYYYTDTKYDYWGQGTQV | | VL | |
| | TVSS | | | |
| 18E2 | QLQLVESGGGLVQPGGSLRLSCAASGFTFSS | 106 | NFMLTQPSALSVTLGQTARITCQGDRLGSRYIYWY | 107 |
| | YVMSWVRQAPGKGLEWVSSIYHDGSHTYYA | | QQKPPQAPVLVIHDDDRRPSGIPERFSGSSSGGTA | |
| | DFVKGRFTISRDNAKNTLYLQMNSLKSEDTAV | | TLTISGAQAEDDGDYYCQSADSSGNPVFGGGTHLT | |
| | YYCASGTSYSGSYYYTDPFFGSWGQGTQVT | | VL | |
| | VSS | | | |
| 14F10 | EVQVQESGGGLVQPGGSLRLSCAASGFTFD | 108 | NFMLTQPSALSVTLGQTAKITCQGGSLGTSYAYWY | 109 |
| | DYDMSWVRQAPGKGLEWVSIISWNGGSTDY | | QQKAGQAPVVVIYDDDNRPSGIPERFSGSSSGGTA | |
| | AESMKGRFTISRDNAKNTLYLQMNSLKSEDT | | TLTISGAQAEDEGDYYCQSEDTSSNFVFGGGTHLT | |
| | AVYFCAKHSVGRNGYDYWGQGTQVTVSS | | VL | |
| 15C3 | ELQLVESGGGLVRPGGSLRLSCAASGFTFSN | 110 | SSELTQASALSVTLGQTAKITCQGGSLGSSYAHWY | 111 |
| | YYMSWVRQAPGKGLEWLSSIYSDGGYTYYA | | QQKPGQAPVLVIYDDDSRPSGIPERFSGSSSGGRA | |
| | DSVKGRFTISRDNAKNTLYLQMNSLKSEDTAV | | TLTISGAQAEDEGDYYCQSADSSGNPVFGGGTKLT | |
| | YYCAKDLKPRNSGTYHDEGYDDWGQGTQVT | | VL | |
| | VSS | | | |
| 15D12 | EVQLVESGGGLVQPGGSLRLSCAASGFTFST | 112 | HSAVTQPSALSVTLGQTAKITCQGGSLGSNYAYWY | 113 |
| | YVMSWVRQAPGKGLEWVSSIYSDGSHTYYA | | QQKPGQAPVLVIYDDDSRPSGIPERFSGSSSGGTA | |
| | DSVKGRFTISRDNAKNTLYLQMNSLKSEDTA | | TLTISGAQAEDEGDYYCQSADSNGNAVFGGGTHLT | |
| | MYYCTTGTSYSGSYYYTDPFFGSWGQGTQVI | | VM | |
| | VSS | | | |
| 17A10 | QVQVQESGPGLVKPSQTLSLTCTVSGGSITT | 114 | QPVLNQLSSMSGSPGQTVTITCTGSSSNIGGGYYL | 115 |
| | SYYAWNWIRQPPGKGLEWMGVIAYDGSTDY | | SWYQQLPGTAPKLLIYNANNRASGVPNRFSGSKTG | |
| | SPSLKSRTSISRDTSKNQFSLQLSSVTPEDTA | | SLASLTITGLQAEDEADYYCGCYDSSLSTPVFGGGT | |
| | VYYCARDYRINSDYAGGYDYWGQGTQVTVS | | KLIVL | |
| | S | | | |
| 17G12 | EVQLVESGGGLVQPGGSLRLSCATSGFTFSS | 116 | SYELTQPSALSVTLGQTAKITCQGGSLGSNYAYWY | 117 |
| | YVMSWVRQAPGKGLEWVSSIYHDGSHTYYA | | QQKPGQAPVLVIYDDDSRPSGIPERFSGSSSGGTA | |
| | DFVKGRFTISRDNAKNTLYLQMNSLKSEDTAV | | TLTISGAQAEDEGDYYCQSADSNGNAVFGGGTHLT | |
| | YYCASGTSYSASYYYTDPFFGSWGQGTQVT | | VL | |
| | VSS | | | |
| 17H2 | QVQVEESGGGLVQPGGSLRLSCAASGFTFS | 118 | SSALTQPSALSVTLGQTADITCQGGSLGGSYAHWY | 119 |
| | SYYMSWVRQAPGKGLEWVSSISSDGSNPYY | | QQKPGQAPMLVIYDDTSRPSGIPERFSGSSSGDRV | |
| | ADSVKGRFTISRDNAKNTLYLQMNSLKSEDTA | | TLTISGAQAEDGGDYYCQSSYSSGNPVFGGGTKLT | |
| | VYYCAKDTLTGASYSDSLYDYWGQGTQVTV | | VL | |
| | SS | | | |
| 19H2 | QVQLVESGGGLVQPGGSLRLSCAASGFTFSS | 120 | SYELTQPSALSVTLRQTAKITCQGDNLGNNYVQWY | 121 |
| | YAMSWVRQAPGKGLEMVSSIYSYSSNTYYAD | | QQKPGQAPELVIYDDNRRPSGIPERFSGSSSGGTA | |
| | SVKGRFTISRDNAKNTLYLQMNSLKSEDTAVY | | TLTISGAQADDEGDYYCQASDSSGNAVVGGGTHLII | |
| | YCAKTTLSRLTYSDYRYDYWGQGTQVTVSS | | L | |
| 20A1 | QLQVVESGGGLVQPGGSLRLSCAASGFTFSS | 122 | QSALTQPSALSVTLGQTAKITCQGGNLGSSYAHWY | 123 |
| | YYMSWVRQAPGKGLEWVSSIYSDGSYAYYA | | QQKPGQAPVLVIYDDDSRPSGIPERFSGSSSGGTA | |
| | DSVKGRFTISRDNAKNTLYLQMNSLKSEDTAV | | TLIISGAQAEDEGDYYCQSADSSGNPVFGGGTKLT | |
| | YYCAKDLKARYSGTYHDEGYDYWGQGTQVT | | VL | |
| | VSS | | | |
| 20D2 | EVQLVESGGGLVQPGGSLRLSCAASGFTFSS | 124 | NFMLTQPSALSVTLGQTAKITCQGGSLGSSYAHWY | 125 |
| | YYMSWVRQAPGKGLERVSSIYSDDSNTDYA | | QQKPGQAPVLVIYDDDSRPSGIPERFSGSSSGGTA | |
| | DSVKGRFTISRDNAKNTLYLQMNSLKSEDTAV | | TLTISGAQAEDEGDYYCQSADSSGNAVFGGGTHLT | |
| | YYCAKATGTVGYYSDYFYDYWGQGTQVTVS | | VL | |
| | S | | | |
| 20G5 | EVQLVESGGGLVQPGGSLRLSCAASGFTFDD | 126 | SSALTQPPSVSGSPGKTVTISCAGTSNDVGYGNYV | 127 |
| | YAMSWVRQAPGKGLEWVSGISWKGGIIYYAE | | SWYQQLPGMAPKLLIYDVNKRASGITDRFSGSKSG | |
| | SMEGRFTISRDNAKNTLYLQMNSLKSEDTAV | | NTASLTISGLQSEDEADYYCASYRTNNNVVFGGGT | |
| | YYCAKALGTVASGQYDYWGQGTQVTVSS | | KVTVL | |
| 21A1 | QLQLVESGGGLVQPGGSLRLSCAASGFTFSS | 128 | SYELTQPSAVSVSLGQTARITCQGDNFGSYYASWY | 129 |
| | YYMSWVRQAPGKGLEWVFSISSDGSNTYYA | | QQKSGQAPVRVIYKDSERPSGIPERFSGSSSGDTA | |
| | DSVKGRFTISRDNAKNTLYLQMNSLKSEDTAV | | TLTISGAQFEDEADYYCLSYDNNGAPVFGGGTKLT | |
| | YYCAKDDNSGSDYEFGYDYWGQGTQVTVSS | | VL | |
| 4D8 | ELQLVESGPGLVKPSQTLSLTCTVSGASITSS | 130 | QAVLTQPPSVSGTLGKTLTISCAGTSSDIGGYNSVS | 131 |
| | YYDWTWIRQPPGKGLEWMGVIHYDGSTYYS | | WYQQLPGTAPKLLIYEVNKRASGIPDRFSGSKSGN | |
| | PSLKSRTSISRDTSKNQFSLQLSSVTPEDTAV | | TASLSISGLQSEDEADYYCASYRNSNNVVFGGGTH | |
| | YYCTQSYSSSPWDYDYWGQGTQVTVSS | | LTVL | |

### Example 2. Further Characterization of Anti-IgE Fab Clones

### A. Sequencing and Reformatting of Fab Clones

The following 8 Fab clones were re-cloned into a human hIgG1 Fc for further characterization: 3D6, 16E4, 3A1, 3D1, 13E4, 18B9, 20D5 and 18E2.

For this purpose, the VH and the VL of each clone were PCR amplified using specific primers, isolated by electrophoresis, purified and digested with restriction enzymes (BsmBi). After digestion and clean-up, ligation of the DNA (VH or VL) was performed into BsmBi pre-digested vectors containing the constant domains of the human lambda or kappa light chain (pUPEX116.08 for Vκ, pUPEX116.09 for Vλ) or of the human IgG1 heavy chain (CH1-CH2-CH3, pUPEX116.07). The transformation of each of the ligated products was done into Top10 bacteria by heat shock and transfer on agarose plate with Ampicillin (resistance gene of the vectors). For each clone (HC and LC), four to eight colonies were picked and sent for sequencing. The clones that showed the proper insert were selected and amplified in order to purify the DNA sequence (MidiPrep).

The production of the 8 human IgG1 antibodies was carried out by transfection of HEK293E cells (using the Polyethylenimine (PEI) with a mix containing the heavy and light chain DNA expression vectors in a 1/1 ratio. After allowing cells to express for 6 days, human monoclonal antibodies were purified from the cell supernatant using the protein-A sepharose beads. Finally, SDS-PAGE analysis was carried out to assess the purity and the integrity of the antibodies.

### B. Characterization of Anti-hIgE Monoclonal Antibodies

ELISA and SPR with a T3000 Biacore were used to assess the binding properties of the anti-hIgE mAbs panel.

i. Binding ELISA

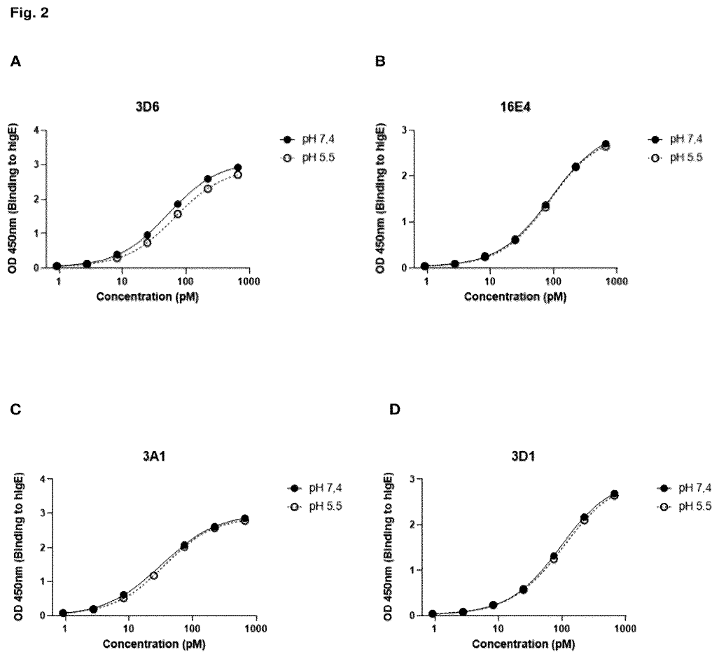

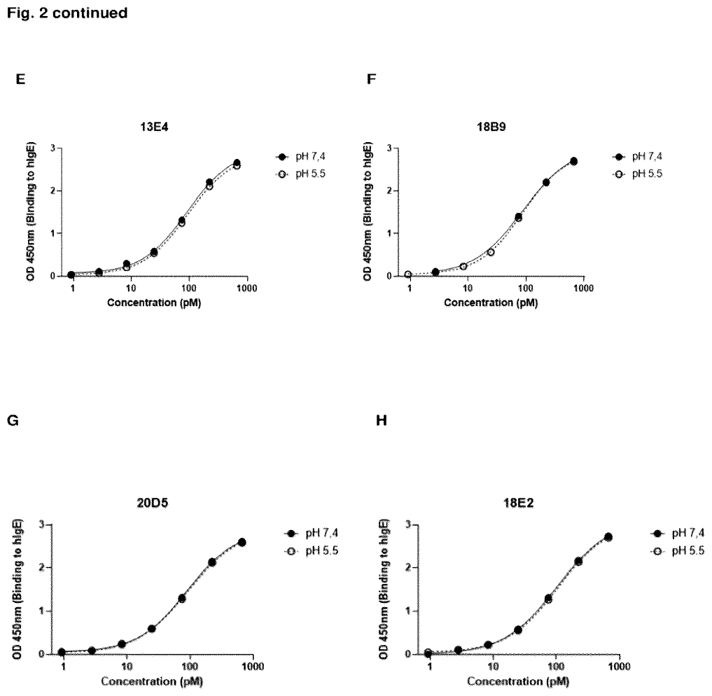

The sequence of hIgE was retrieved from the WGS database. DNA encoding the VH of Motavizumab antibody and constant heavy chain (Cε1-Cε4) of hIgE was synthesized and re-cloned into an expression vector. Together with the Motavizumab light chain, variable and constant human kappa, the IgE vector was transfected into CHO K1 cells, and recombinant Motavizumab human IgE (rMota-hIgE) was produced. hIgE was purified using MabSelect™ SuRe™. rMota-hIgE was used to assess the relative binding properties of the 8 anti-hIgE mAbs by ELISA. Briefly a Maxisorp plate was coated with recombinant human respiratory syncytial virus protein F (RSV-F) (0.5 μg/mL), then blocked with PBS with 3% BSA and 0.05% Tween. 1 μg/ml of rMota-hIgE was captured before being incubated with a serial dilution of the anti-hIgE mAbs. After several washing steps at pH 7.4 or pH 5.5, detection of the bound mAbs was carried out with an anti-human Fc-HRP antibody. Absorbance was measured at 450 nM (reference at 620 nm) with Tecan instrument. All re-cloned antibodies were able to bind human IgE (see FIG. 2).

ii. Competition ELISA

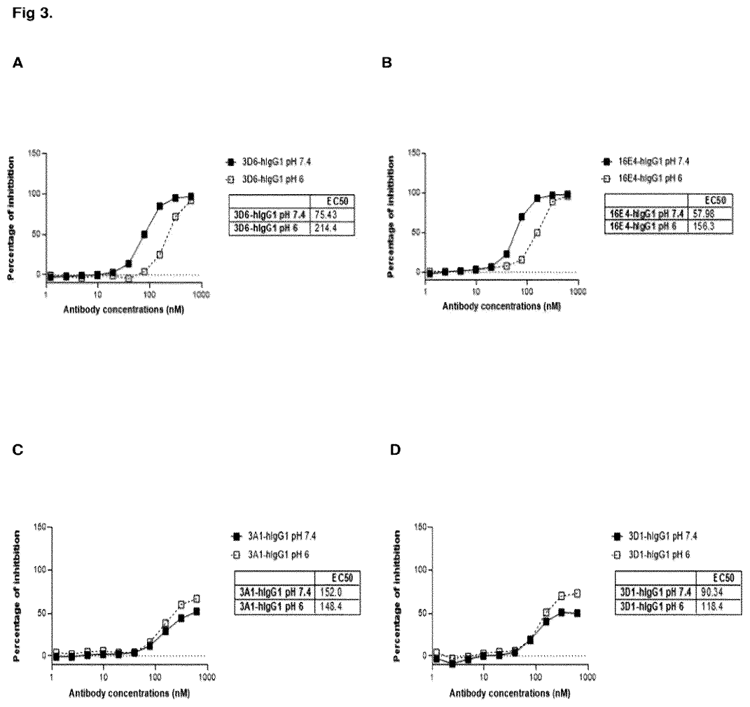

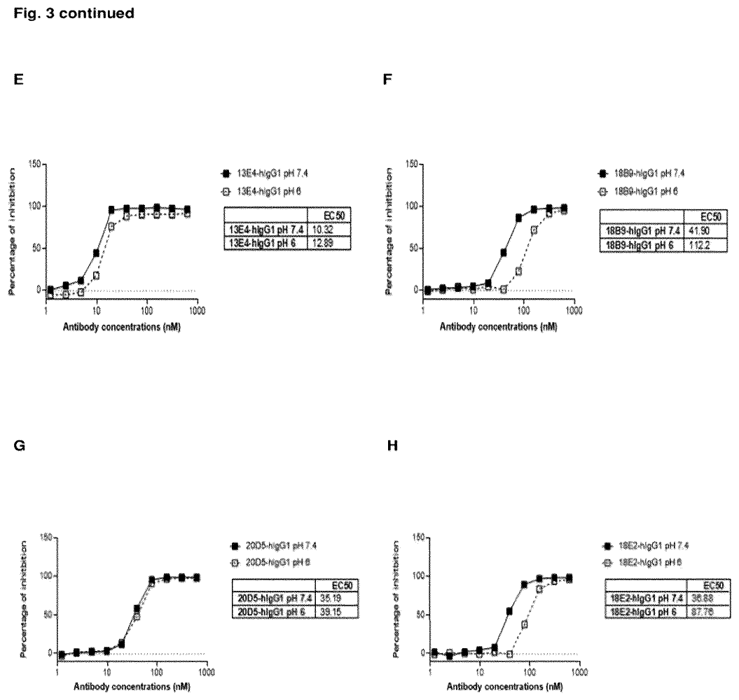

In the exact set-up to that used during the initial screening, the inhibition hIgE binding to hFcεRIα by 8 different anti-hIgE antibodies was analyzed by ELISA at pH 7.4 and pH 6. The raw data (OD values) were plotted on GraphPad Prism 7.01. The IC50 values of each compound were calculated with a non-linear regression (log(agonist) vs. response Variable slope (four parameters)). The results are shown in FIG. 3 and Table 14. The clone 13E4 showed the highest affinity to hIgE at pH 7.4. Three clones showed the highest pH-dependent differential affinity to hIgE: 3D6, 16E4 and 18B9.

TABLE 14
IC50 (nM) of inhibition of hIgE binding to FcεRI by ELISA

| | IC50 (nM) | IC50 (nM) | |
|---|---|---|---|
| Antibody | (pH 7.4) | (pH 6) | Ratio pH 6/7.4 |
| 3D6 | 75.43 | 214.4 | 2.8 |
| 16E4 | 57.98 | 156.3 | 2.7 |
| 3A1 | 152 | 148.4 | 1.0 |
| 3D1 | 90.34 | 118.4 | 1.3 |
| 13E4 | 10.32 | 12.89 | 1.2 |
| 18B9 | 41.9 | 112.2 | 2.7 |
| 20D5 | 35.19 | 39.15 | 1.1 |
| 18E2 | 36.88 | 87.76 | 2.4 |

iii. SPR Analysis: Competition of IgE Binding to FcεRI

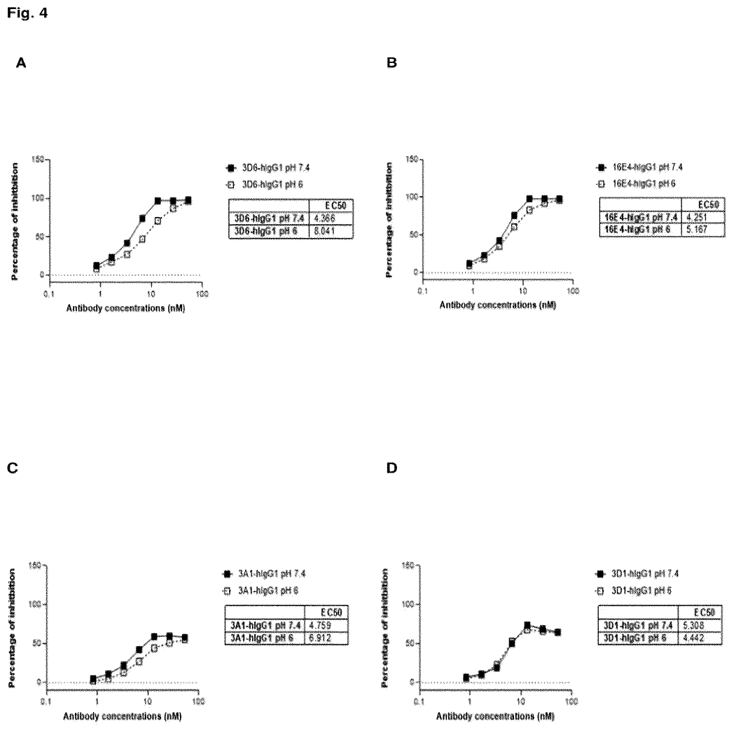

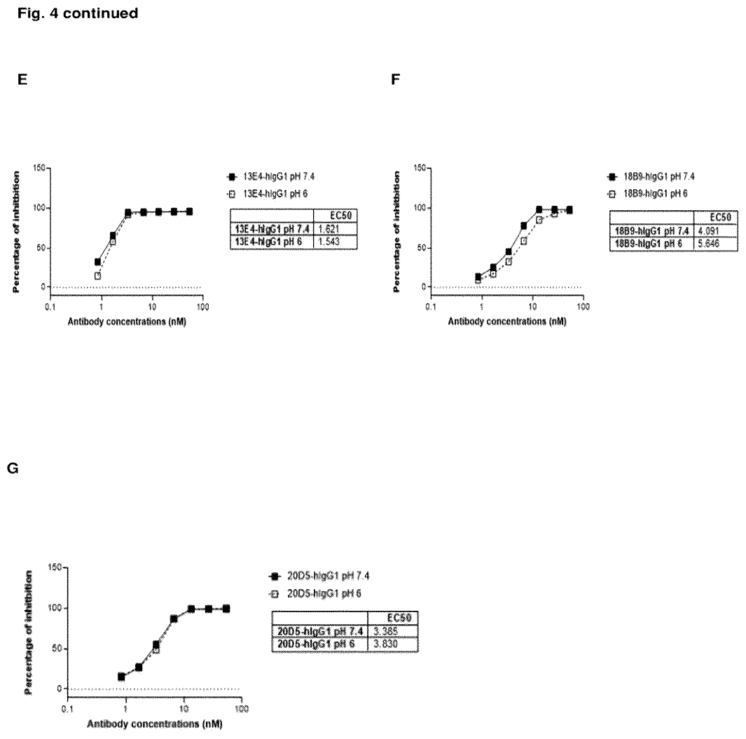

The binding capacity of the human anti-hIgE IgG1 mAbs was analyzed on Biacore T3000. For this purpose, a competition approach was set-up. A CM5 Chip was coated with hFcεRIa at 1500 RU. A fixed concentration of hIgE (1 μg/mL) was pre-incubated with serial concentrations of the human IgG1 antibody panel before being injected to the Chip coated with h hFcεRIa. The assay was performed in HBS-EP pH7.4 or HBS-EP pH 5.5. Raw data were analyzed via BIA evaluation software with a blank subtraction (2-1). The RU values were plotted on GraphPad Prism 7.01. The IC50 values of each compound were calculated with a non-linear regression (log(agonist) vs. response Variable slope (four parameters)). The results are shown in FIG. 4 and Table 15 below. As observed in competition ELISA, the antibody with the highest potency is the clone 13E4. In this approach, the clone 3D6 showed the highest pH dependency.

TABLE 15
IC50 (nM) of inhibition of hIgE binding to FcεRI by Biacore

| | IC50 (nM) | IC50 (nM) | |
|---|---|---|---|
| Antibody | (pH 7.4) | (pH 6) | Ratio pH 6/7.4 |
| 3D6 | 4.366 | 8.041 | 1.8 |
| 16E4 | 4.251 | 5.167 | 1.2 |
| 3A1 | 4.759 | 6.912 | 1.5 |
| 3D1 | 5.308 | 4.442 | 0.8 |
| 13E4 | 1.621 | 1.543 | 1.0 |
| 18B9 | 4.091 | 5.646 | 1.4 |
| 20D5 | 3.385 | 3.830 | 1.1 |
| 18E2 | NA | NA | |

### C. Identification of Clones Cross-Reactive to Cynomolgus Monkey IgE (cIgE)

The sequence of cIgE was retrieved from the WGS database. The sequence showed 85% identity on the full Fc (Cε1-Cε4). DNA encoding the VH of Motavizumab antibody and constant heavy chain (Cε1-Cε4) of cIgE was synthesized and re-cloned into an expression vector. The DNA encoding the VL of Motavizumab was cloned into an expression vector containing the Vkappa constant region. The plasmids were transfected into CHO K1 cells. cIgE was purified using MabSelect™ SuRe™. ELISA and SPR with a T3000 Biacore were used to assess the cross reactivity of the anti-hIgE mAbs panel.

i. Binding ELISA

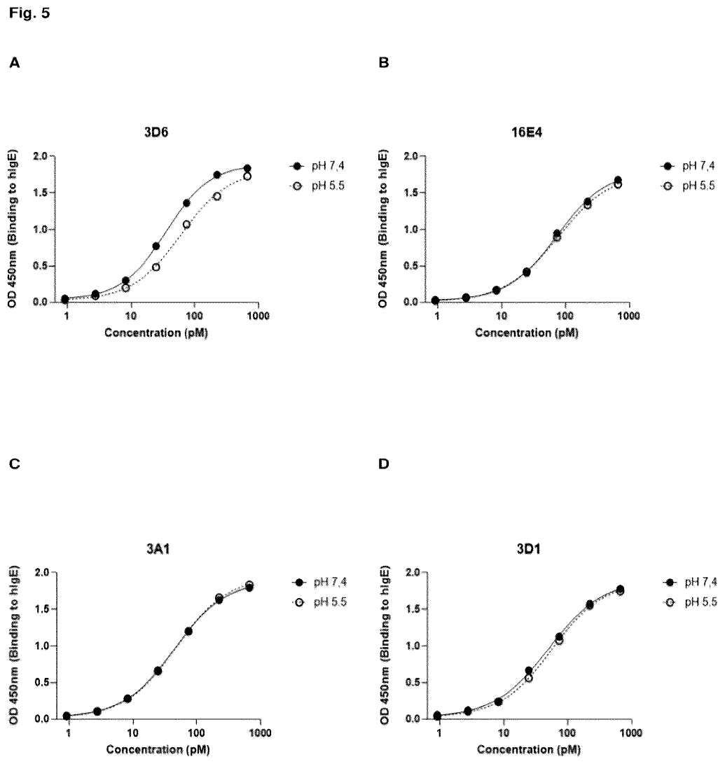

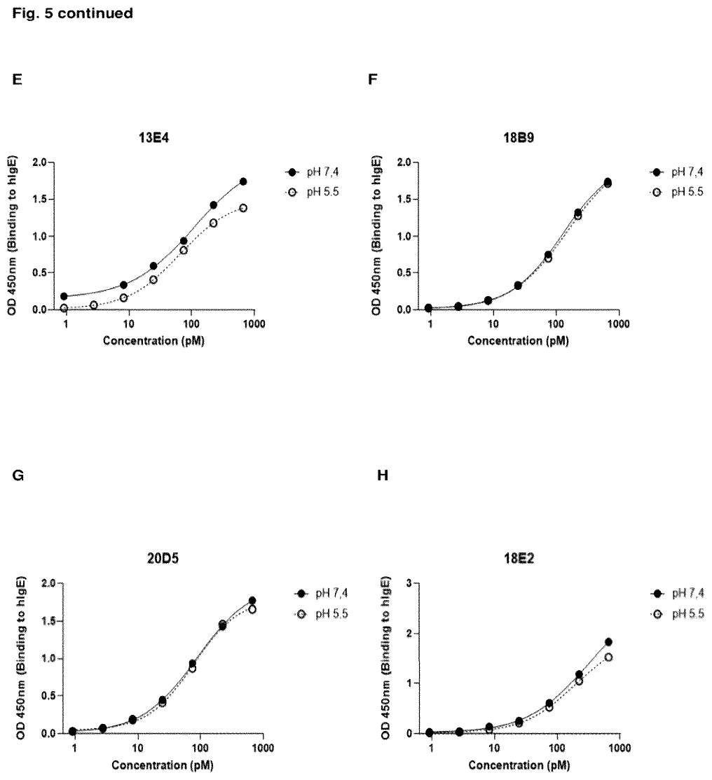

In a similar set-up to that used for hIgE, the relative binding properties of the 8 anti-hIgE mAbs were analyzed by ELISA. Briefly a Maxisorp plate was coated with RSV-F (0.5 μg/mL), then blocked with PBS with 3% BSA and 0.05% Tween. 1 μg/ml of rMota-cIgE was captured before being incubated with a serial dilution of the anti-hIgE mAbs. After several washing steps at pH 7.4 or pH 5.5, detection of the bound mAbs was done with an anti-human Fc-HRP antibody. Absorbance was measured at 450 nM (reference at 620 nm) with Tecan instrument. Finally, the raw data (OD values) were plotted on GraphPad Prism 7.01. The 8 clones are able to bind to cIgE with various affinities (see FIG. 5). The clones 3D6 and 13E4 have a pH dependent binding affinity to cIgE.

### Example 3. Identification of PH-Dependent Anti-IgE Antibodies Blocking IgE/FcεRIα Interaction by Histidine Engineering

### A. Engineering PH-Dependent IgE Binding

Three antibodies were selected in order to increase the pH dependency. Histidine mutations were introduced into the CDR sequences by rational selection of the position to mutate as described in WO2018/206748, incorporated herein by reference.

The CDR, VH and VL sequences of mutated clones are shown in Tables 16, 17 and 18 below.

TABLE 16
Heavy chain CDR sequences of antibodies produced in order to
increase the pH dependent affinity to hIgE

| Antibody | | | | SEQ ID | | SEQ ID |
|---|---|---|---|---|---|---|
| clone | CDR1 | SEQ ID NO. | CDR2 | NO. | CDR3 | NO. |
| 18E2 | SYVMS | 20 | SIYHDGSHTYYADFVKG | 21 | GTSYSGSYYYTDPFFGS | 22 |
| VH18E2_S35H | SYVMH | 132 | SIYHDGSHTYYADFVKG | 21 | GTSYSGSYYYTDPFFGS | 22 |
| 18B9 | SYVMS | 20 | SIYSDGSHTYYADSVKG | 23 | NLEHYSGSYYYTDPRYDY | 24 |
| VH18B9_S35H | SYVMH | 133 | SIYSDGSHTYYADSVKG | 23 | NLEHYSGSYYYTDPRYDY | 24 |
| 3D1 | SYYMS | 12 | SIYSDGRGSKTFYADSVKG | 18 | DLLVAARGSM | 19 |
| VH3D1_S35H | SYYMH | 134 | SIYSDGRGSKTFYADSVKG | 18 | DLLVAARGSM | 19 |

TABLE 17
Light chain CDR sequences of antibodies produced in order to
increase the pH dependent affinity to hIgE

| Antibody | | | | SEQ ID | | SEQ ID |
|---|---|---|---|---|---|---|
| clone | CDR1 | SEQ ID NO. | CDR2 | NO. | CDR3 | NO. |
| 18E2 | QGDRLGSRYIY | 68 | DDDRRPS | 69 | QSADSSGNPV | 56 |
| VL18E2_Y34H | QGDRLGSRYIH | 135 | DDDRRPS | 69 | QSADSSGNPV | 56 |
| 18B9 | QGGSLGSSYVH | 64 | DGDSRPS | 65 | QSADSSGNAV | 66 |
| VL18B9_Y49H | QGGSLGSSYVH | 64 | DGDSRPS | 65 | QSADSSGNAV | 66 |
| 3D1 | QGGTLGSYGAH | 59 | GDNSRPS | 60 | QSFDYSGNAV | 61 |
| VL3D1_Y49H | QGGTLGSYGAH | 59 | GDNSRPS | 60 | HSFDYSGNAV | 136 |
| VL3D1_Q89H | | | | | | |

TABLE 18
VH and VL sequences of antibodies selected
in order to increase the pH dependent affinity to hIgE

| Antibody | | | | |
|---|---|---|---|---|
| clone | VH | SEQ ID NO. | VL | SEQ ID NO. |
| 18E2 | QLQLVESGGGLVQPGGSLRLSCAA | 106 | NFMLTQPSALSVTLGQTARITCQGDRLG | 107 |
| | SGFTFSSYVMSWVRQAPGKGLEWV | | SRYIYWYQQKPPQAPVLVIHDDDRRPSGI | |
| | SSIYHDGSHTYYADFVKGRFTISRD | | PERFSGSSSGGTATLTISGAQAEDDGDY | |
| | NAKNTLYLQMNSLKSEDTAVYYCAS | | YCQSADSSGNPVFGGGTHLTVL | |
| | GTSYSGSYYYTDPFFGSWGQGTQV | | | |
| | TVSS | | | |
| VH18E2_S35H | QLQLVESGGGLVQPGGSLRLSCAA | 137 | NFMLTQPSALSVTLGQTARITCQGDRLG | 107 |
| | SGFTFSSYVMHWVRQAPGKGLEW | | SRYIYWYQQKPPQAPVLVIHDDDRRPSGI | |
| | VSSIYHDGSHTYYADFVKGRFTISRD | | PERFSGSSSGGTATLTISGAQAEDDGDY | |
| | NAKNTLYLQMNSLKSEDTAVYYCAS | | YCQSADSSGNPVFGGGTHLTVL | |
| | GTSYSGSYYYTDPFFGSWGQGTQV | | | |
| | TVSS | | | |
| VL18E2_Y34H | QLQLVESGGGLVQPGGSLRLSCAA | 106 | NFMLTQPSALSVTLGQTARITCQGDRLG | 138 |
| | SGFTFSSYVMSWVRQAPGKGLEWV | | SRYIHWYQQKPPQAPVLVIHDDDRRPSG | |
| | SSIYHDGSHTYYADFVKGRFTISRD | | IPERFSGSSSGGTATLTISGAQAEDDGDY | |
| | NAKNTLYLQMNSLKSEDTAVYYCAS | | YCQSADSSGNPVFGGGTHLTVL | |
| | GTSYSGSYYYTDPFFGSWGQGTQV | | | |
| | TVSS | | | |
| 18E2VH_S3 | QLQLVESGGGLVQPGGSLRLSCAA | 137 | NFMLTQPSALSVTLGQTARITCQGDRLG | 138 |
| 5H_VL_Y34H | SGFTFSSYVMHWVRQAPGKGLEW | | SRYIHWYQQKPPQAPVLVIHDDDRRPSG | |
| | VSSIYHDGSHTYYADFVKGRFTISRD | | IPERFSGSSSGGTATLTISGAQAEDDGDY | |
| | NAKNTLYLQMNSLKSEDTAVYYCAS | | YCQSADSSGNPVFGGGTHLTVL | |
| | GTSYSGSYYYTDPFFGSWGQGTQV | | | |
| | TVSS | | | |
| 18B9 | QVQLVESGGGLVQPGGSLRLSCAA | 102 | SSALTQPSALSVTLGQTAKITCQGGSLGS | 103 |
| | SGFTFSSYVMSWVRQAPGKGLEWV | | SYVHWYQQKPGQAPVLVIYDGDSRPSGI | |
| | SSIYSDGSHTYYADSVKGRFTISRD | | PERFSGSSSGGTATLTISGAQAEDEDDY | |
| | NAKNTLYLQMNSLKSEDTAVYYCAK | | YCQSADSSGNAVFGGGTHLTVL | |
| | NLEHYSGSYYYTDPRYDYWGQGTQ | | | |
| | VTVSS | | | |
| VH18B9_S35H | QVQLVESGGGLVQPGGSLRLSCAA | 139 | SSALTQPSALSVTLGQTAKITCQGGSLGS | 103 |
| | SGFTFSSYVMHWVRQAPGKGLEW | | SYVHWYQQKPGQAPVLVIYDGDSRPSGI | |
| | VSSIYSDGSHTYYADSVKGRFTISRD | | PERFSGSSSGGTATLTISGAQAEDEDDY | |
| | NAKNTLYLQMNSLKSEDTAVYYCAK | | YCQSADSSGNAVFGGGTHLTVL | |
| | NLEHYSGSYYYTDPRYDYWGQGTQ | | | |
| | VTVSS | | | |
| VL18B9_Y49H | QVQLVESGGGLVQPGGSLRLSCAA | 102 | SSALTQPSALSVTLGQTAKITCQGGSLGS | 140 |
| | SGFTFSSYVMSWVRQAPGKGLEWV | | SYVHWYQQKPGQAPVLVIHDGDSRPSGI | |
| | SSIYSDGSHTYYADSVKGRFTISRD | | PERFSGSSSGGTATLTISGAQAEDEDDY | |
| | NAKNTLYLQMNSLKSEDTAVYYCAK | | YCQSADSSGNAVFGGGTHLTVL | |
| | NLEHYSGSYYYTDPRYDYWGQGTQ | | | |
| | VTVSS | | | |
| 18B9_VH_S3 | QVQLVESGGGLVQPGGSLRLSCAA | 139 | SSALTQPSALSVTLGQTAKITCQGGSLGS | 140 |
| 5H_VL_Y49H | SGFTFSSYVMHWVRQAPGKGLEW | | SYVHWYQQKPGQAPVLVIHDGDSRPSGI | |
| | VSSIYSDGSHTYYADSVKGRFTISRD | | PERFSGSSSGGTATLTISGAQAEDEDDY | |
| | NAKNTLYLQMNSLKSEDTAVYYCAK | | YCQSADSSGNAVFGGGTHLTVL | |
| | NLEHYSGSYYYTDPRYDYWGQGTQ | | | |
| | VTVSS | | | |
| 3D1 | QLQVVESGGGLVQPGGSLRLSCAA | 98 | NFMLTQPSAVSVSLEQTARITCQGGTLG | 99 |
| | SGFTFSSYYMSWVRQAPGKGLEWV | | SYGAHWYQQKPGQAPVLLIYGDNSRPS | |
| | SSIYSDGRGSKTFYADSVKGRFTISR | | GIPERFSGTRSGGTATLTISGAQAEDEAD | |
| | DNAKNTLYLQMNSLKSEDTAVYFCA | | YYCQSFDYSGNAVFGGGTHLTVL | |
| | KDLLVAARGSMDYWGQGTQVTVSS | | | |
| VH3D1_S35H | QLQVVESGGGLVQPGGSLRLSCAA | 141 | NFMLTQPSAVSVSLEQTARITCQGGTLG | 99 |
| | SGFTFSSYYMHWVRQAPGKGLEW | | SYGAHWYQQKPGQAPVLLIYGDNSRPS | |
| | VSSIYSDGRGSKTFYADSVKGRFTIS | | GIPERFSGTRSGGTATLTISGAQAEDEAD | |
| | RDNAKNTLYLQMNSLKSEDTAVYFC | | YYCQSFDYSGNAVFGGGTHLTVL | |
| | AKDLLVAARGSMDYWGQGTQVTVS | | | |
| | S | | | |
| VL3D1_Y49H | QLQVVESGGGLVQPGGSLRLSCAA | 98 | NFMLTQPSAVSVSLEQTARITCQGGTLG | 142 |
| VL3D1_0389H | SGFTFSSYYMSWVRQAPGKGLEWV | | SYGAHWYQQKPGQAPVLLINGDNSRPS | |
| | SSIYSDGRGSKTFYADSVKGRFTISR | | GIPERFSGTRSGGTATLTISGAQAEDEAD | |
| | DNAKNTLYLQMNSLKSEDTAVYFCA | | YYCHSFDYSGNAVFGGGTHLTVL | |
| | KDLLVAARGSMDYWGQGTQVTVSS | | | |

SPR with a T3000 Biacore was used to assess the binding capacity of the mutated clones. The previously described competition approach was used. Raw data were analyzed via BIA evaluation software with a blank subtraction (2-1). The RU values were plotted on GraphPad Prism 7.01. The IC50 values of each compound, calculated with a non-linear regression (log(agonist) vs. response Variable slope (four parameters)). The results are shown in table 19 below.

TABLE 19
IC50 of pH dependent engineered anti-hIgE clones.

| | IC50 μg/ml | IC50 μg/ml |
|---|---|---|
| | (pH 7.4) | (pH 5.5) |
| 18E2 | 0.628 | 0.484 |
| VH18E2_S35H | 0.811 | 3.729 |
| VL18E2_Y34H | 1.102 | 5.183 |
| 18E2_VH_S35H_VL_Y34H | 1.562 | 9.856 |
| 18B9 | 0.662 | 1.073 |
| VH18B9_S35H | 21.670 | 22.280 |
| VL18B9_Y49H | 0.749 | 4.057 |
| 18B9_VH_S35H_VL_Y49H | 26.260 | 24.630 |
| 3D1 | 1.363 | 11.660 |
| VH3D1_S35H | 15.540 | 23.040 |
| VL3D1_Y49H | 4.410 | 15.170 |
| VL3D1_Q89H | | |

The results can be summarised as follows:
Mutation S35H does not affect IgE binding at pH 7.4 but increases pH dependency in clone 18E2. On the contrary, this mutation abrogates IgE binding of clones 18B9 and 3D1.
For clone 18E2, the best pH dependent binder is 18E2_VH_S35H_VL_Y34H with a ratio of 6.3 between IC50 at pH5.5 and pH 7.4. However, the affinity at pH 7.4 is reduced by 2.5-fold compare to the WT clone.
For clone 18B9, the best pH dependent binder is VL18B9_Y49H with a ratio of 5.4 between IC50 at pH5.5 and pH 7.4. The affinity at pH 7.4 is not affected.
The clone WT 3D1 showed the best pH dependent affinity with a ratio of 8.6 between IC50 at pH5.5 and pH 7.4. His mutation affects IgE binding at pH 7.4 and does not increase the ratio between IC50 at pH5.5 and pH 7.4 compared to the WT antibody.

### Example 4. Production and Characterization of Anti-hIgE-ABDEG Antibodies

A. Reformatting anti-IgE Fab into human IgG1 Fc-ABDEG™ human IgG1 Fc-LALA-ABDEG™

Three Fab clones: 13E4; 18E2_VH_S35H_VL_Y34H (18E2His2); and VL18B9_Y49H (18B9His), were re-cloned into a human hIgG1 Fc containing ABDEG™ mutations. For this purpose, DNA strings of the VH of each clone containing BsmBI restriction sites were ordered. After digestion and clean-up, ligation of the DNA was performed into BsmBI pre-digested vectors containing the constant domains of the human IgG1 heavy chain with ABDEG™ mutation (CH1-CH2-CH3, pUPEX32a), or human IgG1 heavy chain with LALA and ABDEG™ mutation (CH1-CH2-CH3, pUPEX94). The transformation of each of the ligated products was done into Top10 bacteria by heat shock and transfer of the transformed bacteria on agarose plate with Ampicillin (resistance gene of the vectors). Per clones (HC and LC), four to eight colonies were picked and sent for sequencing. The clones that showed the proper insert were selected and amplified in order to purify the DNA sequence (MidiPrep).

The production of the 3 human IgG1-ABDEG™ antibodies was done by transfection with a ratio of 1 heavy chain for 1 light chain incorporated in HEK293E cells via the Polyethylenimine (PEI). After 6 days, human monoclonal antibodies were purified from the cell supernatants using protein-A sepharose beads. Finally, SDS-PAGE analysis was done to assess the purity and the integrity of the antibodies (150 kDa).

### B. Characterization of Anti-IgE-ABDEG™ Antibodies

ELISA and SPR with a T3000 Biacore were used to assess the binding properties of the anti-hIgE-ABDEG™ mAbs.

i. IgE Binding ELISA

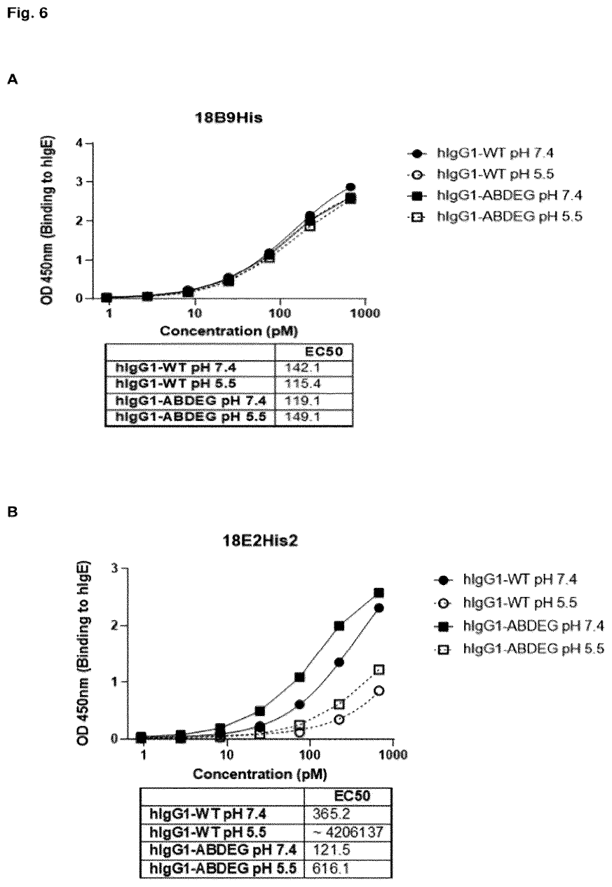

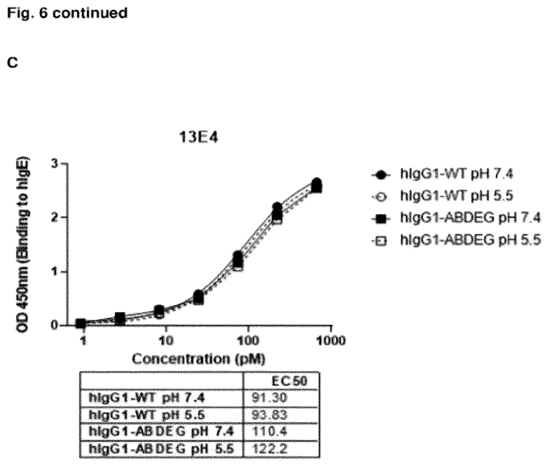

In the exact set-up to that used above, the relative binding properties of the 3 anti-hIgE-ABDEG™ antibodies were analyzed by ELISA. The raw data (OD values) were plotted on GraphPad Prism 7.01 (see FIG. 6).
All 3 clones were able to bind hIgE and compete with FcεRIA for binding to hIgE
Clone 13E4 had the highest affinity to hIgE.
Clone 18E2His2 showed the highest pH dependency.
   ii. IgE Competition ELISA

In the exact set-up to that used above, the inhibition hIgE binding to hFcεRIα by anti-hIgE-ABDEG™ antibodies was analyzed by ELISA at pH 7.4 and pH 6. The raw data (OD values) were plotted on GraphPad Prism 7.01. The IC50 values of each compound were calculated with a non-linear regression (log(agonist) vs. inhibition Variable slope (four parameters)). The results are shown in table 20 below.

TABLE 20
IC50 of IgE competition ELISA.
ABDEG ™ function does not affect IgE binding

| | IC50 (ng/ml) | IC50 (ng/ml) |
|---|---|---|
| | (pH 7.4) | (pH 6) |
| 18B9His-hIgG1-WT | 190 | 3017 |
| 18B9His-hIgG1-ABDEG | 130 | 2547 |
| 18E2His2-hIgG1-WT | 2482 | 77189 |
| 18E2His2-hIgG1-ABDEG | 1494 | 26592 |
| 13E4-hIgG1-WT | 6.815 | 11.42 |
| 13E4-hIgG1-ABDEG | 6.502 | 10.65 |

The 3 clones were able to inhibit IgE:FcεRIα interaction
Clone 13E4 was the most potent clone to inhibit IgE:FcεRIα interaction.
   iii. SPR Analysis for Competition of IgE:FcεRIα Interaction

As described above, the binding capacity of the human IgG1 mAbs anti-hIgE was analyzed on Biacore T3000 using a competition approach. The assay was performed in HBS-EP pH7.4 or HBS-EP pH 5.5. Raw data were analyzed via BIA evaluation software with a blank subtraction (2-1). The RU values were plotted on GraphPad Prism 7.01. The IC50 values of each compound, calculated with a non-linear regression (log(agonist) vs. response Variable slope (four parameters)). The results are shown in table 21 below. The results obtained confirmed data obtained with the competition ELISA.

TABLE 21
IC50 of IgE competition, SPR analysis

| | IC50 (μg/ml) | IC50 (μg/ml) |
|---|---|---|
| | (pH 7.4) | (pH 6) |
| 18B9His-hIgG1-WT | 2.7 | 58.5 |
| 18B9His-hIgG1-ABDEG | 2.9 | 52.9 |
| 18E2His2-hIgG1-WT | 6.0 | 6312 |
| 18E2His2-hIgG1-ABDEG | 5.8 | 7323 |
| 13E4-hIgG1-WT | 2.3 | 2.5 |
| 13E4-hIgG1-ABDEG | 3.0 | 3.2 |

The 3 clones were able to inhibit IgE:FcεRIα interaction. The most potent clone was the clone 13E4, whereas the clone with the highest binding pH-dependency was 18E2His2.

iv. FcRn Binding ELISA

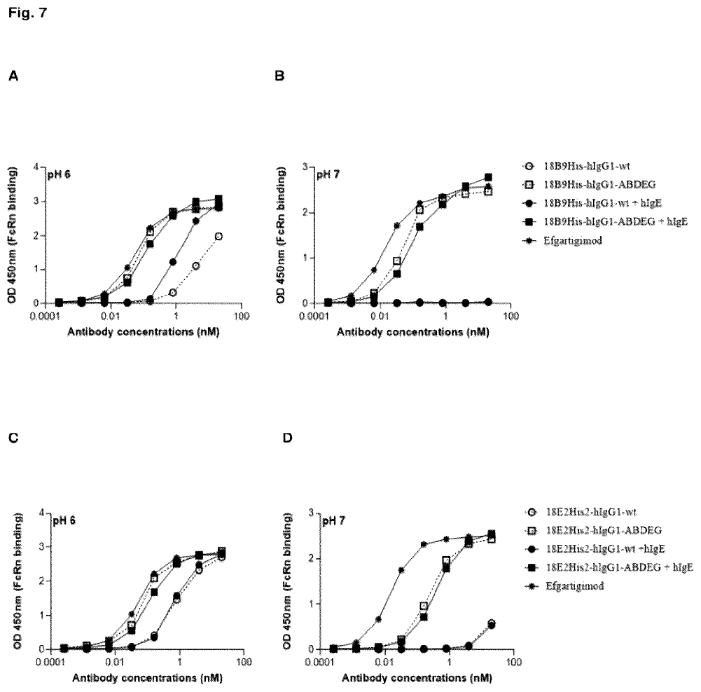

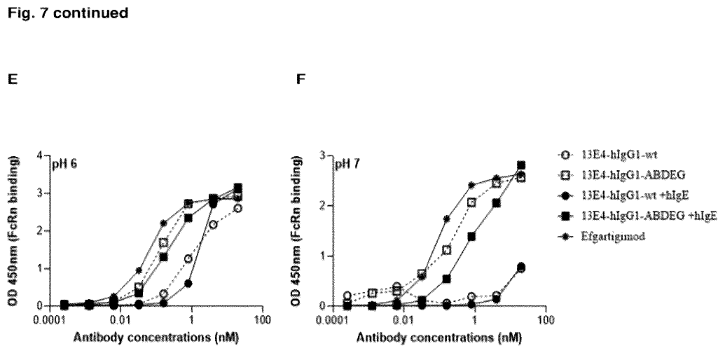

In order to test the binding capacity of the full antibodies equipped with ABDEG™ to FcRn, an ELISA binding assay was established. Briefly, a Maxisorp plate was coated with neutravidin (1 μg/mL, ThermoFisher Cat #31000), then was blocked with PBS1% Casein. Biotinylated human FcRn (0.5 μg/ml, ImmuniTrack, cat #ITF01) was added, before incubation with serial dilutions of anti-hIgG1-ABDEG antibodies pre-incubated or not with hIgE. Detection of binders was done with a Goat F(ab′)2 anti-Human IgG-Fc-HRP (1/20,000, Abcam cat #ab98595). The assay was performed at pH 6 and pH7. Absorbance was measured at 450 nm (reference at 620 nm) with Tecan instrument. The results are show in FIG. 7. Antibodies reformatted in human IgG1 Fc equipped with ABDEG™ mutation had higher affinity to FcRn at pH 6 and pH 7 than human IgG1 Fc WT.

v. IgG3 Competition ELISA

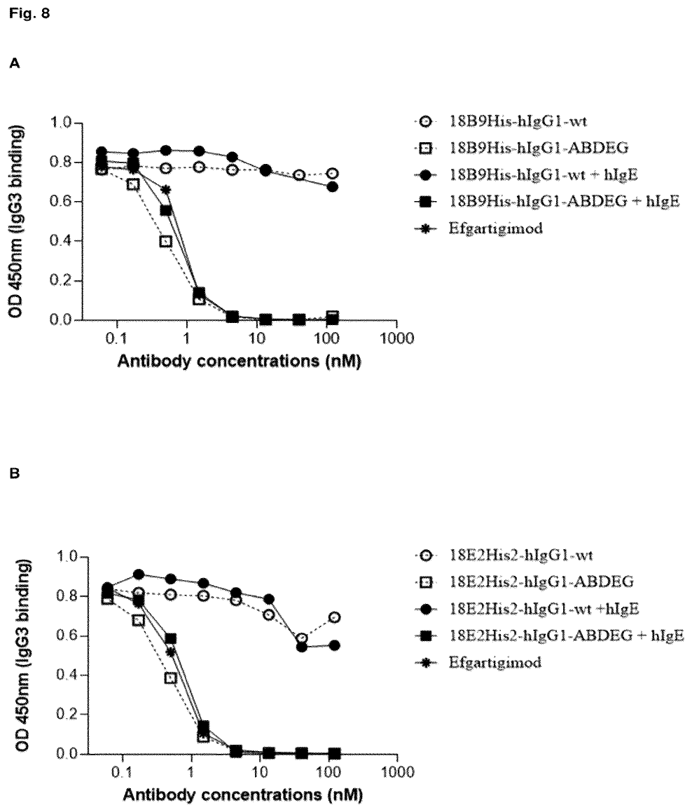

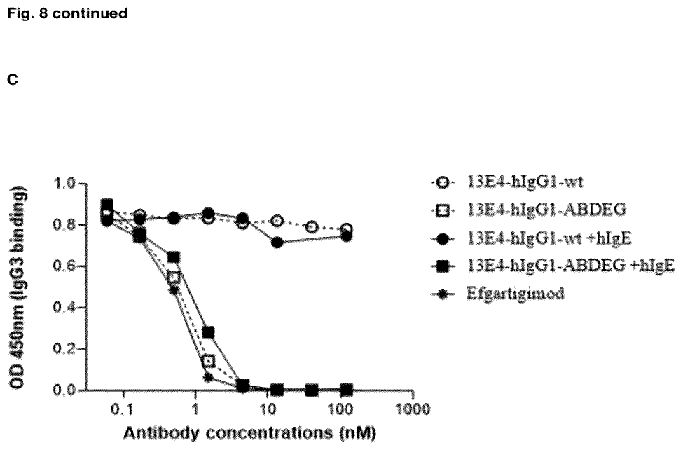

In order to test the functionality of ABDEG™ in full antibodies equipped with ABDEG™ in an in vitro assay, a competition ELISA binding assay was established. In short, a Maxisorp plate was coated with neutravidin (1 μg/mL, ThermoFisher Cat #31000), then was blocked with PBS1% Casein. A mix of biotinylated human FcRn (0.5 μg/ml, ImmuniTrack, cat #ITF01), recombinant hIgG3 (in house production) and serial dilutions of anti-hIgG1-ABDEG antibodies, pre-incubated or not with hIgE, was added to the plate. Detection of bound IgG3 was done with a mouse anti-human IgG3 (ThermoFisher Cat #MH1732) Goat F(ab′)2 anti-Human IgG-Fc-HRP (1/20,000, Abcam cat #ab98595). The assay was performed at pH 6. Absorbance was measured at 450 nm (reference at 620 nm) with Tecan instrument. The results are show in FIG. 8.

### Example 5. Inhibition of IgE Binding to FcεRI+ Cells

The ability of the anti-hIgE-ABDEG™ antibodies to inhibit IgE binding to hFcεRIα+ cells was analysed. Bone marrow cells were isolated from Tg hIgE/hFcεRIα mice. These cells were differentiated in vitro into mast cells in the presence of murine IL-3 for 30 days. The bone-marrow derived mast cells were incubated with human IgE in presence of serial dilutions anti-IgE-ABDEG™ mAbs. The residual hIgE binding was measured by flow cytometry. Median fluorescence intensity, calculated using FlowJo software, were plotted on GraphPad Prism 7.01.

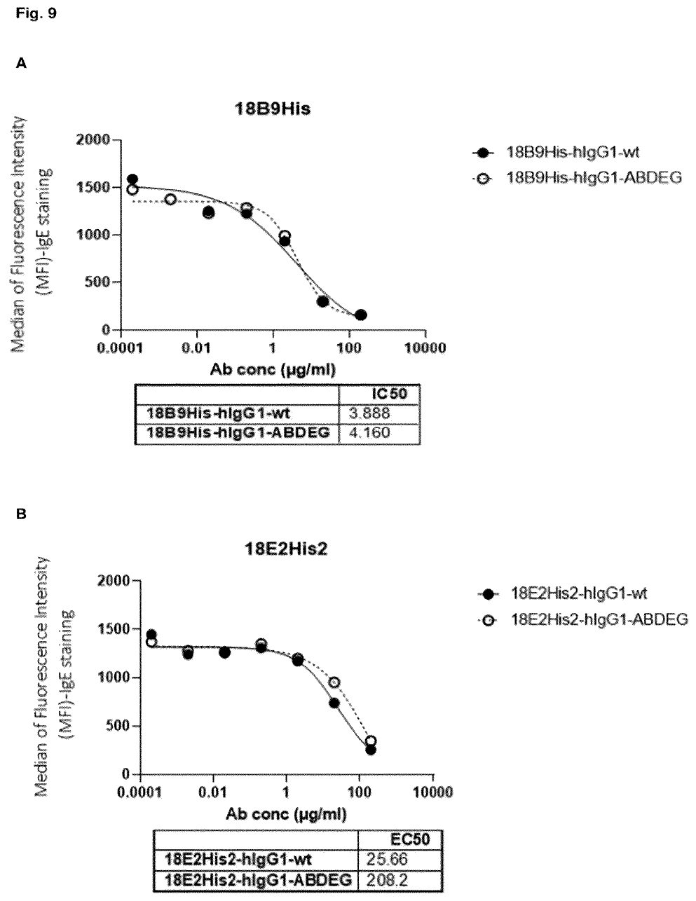

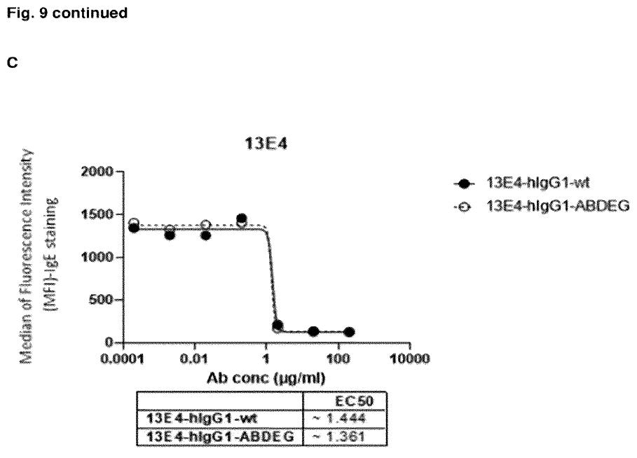

The IC50 values of each compound were calculated with a non-linear regression (log(agonist) vs. inhibition Variable slope (four parameters)). The results are shown in FIG. 9.
The 3 clones were able to inhibit hIgE binding to hFcεRIα+ cells.
Clone 13E4 displayed the highest potency.
ABDEG™ mutations in the Fc fragment do not affect the anti-IgE function of different clones.

### Example 6. Anti-IgE Antibodies Binding to IgE Pre-Bound on FcεRI+ Cells

### A. IgE Crosslinking ELISA

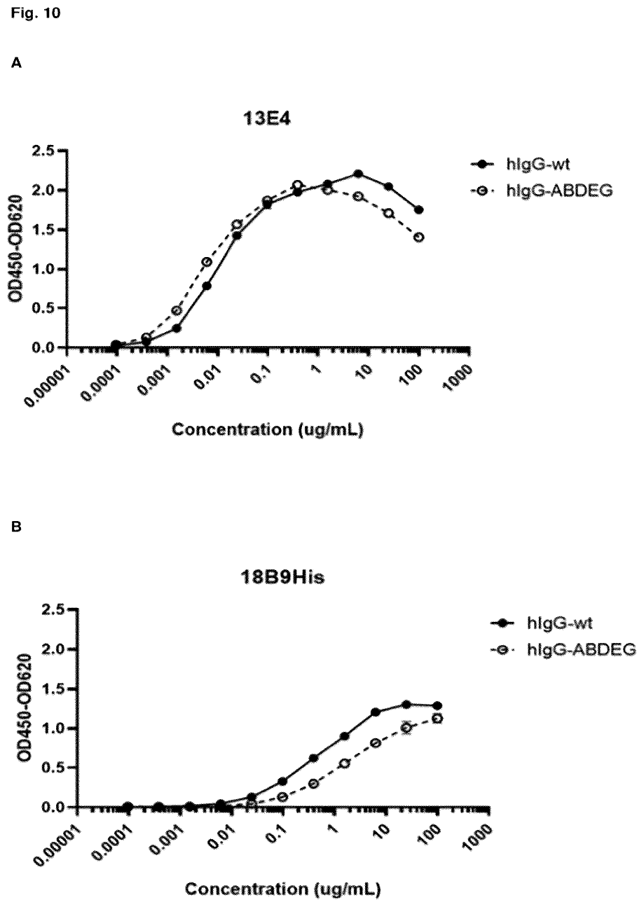

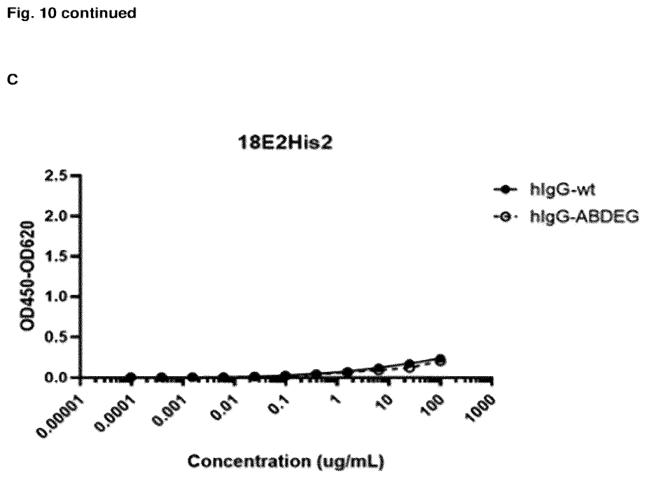

Antibody binding to human IgE associated with FcεRIα was analyzed by ELISA. Briefly a Maxisorp plate was coated with hFcεRIα (0.5 μg/mL), then blocked with PBS with 1% BSA and 0.05% Tween. 3 μg/ml of rMota-hIgE was captured before being incubated with a serial dilution of the anti-hIgE mAbs. After several washing steps, detection of the bound mAbs was done with an anti-human Fc-HRP antibody. Absorbance was measured at 450 nM (reference at 620 nm) with Tecan instrument. Finally, the raw data (OD values) were plotted on GraphPad Prism 7.01 (see FIG. 10).
The clones 13E4 and 18B9His were able to bind IgE associated to hFcεRIα+
The clone 18E2His2 does not bind IgE associated to hFcεRIα+

### B. Basophil Activation Test

Antibody binding to human IgE pre-bound on human basophils was analyzed by flow cytometry. Blood was obtained from a house dust-mite allergic donor. Basophil activation was measured according to FLOW CAST® Kit (BUHLMANN) in presence of anti-hIgE-ABDEG™ antibodies. The results were analyzed by flow cytometry and raw data were processed using FlowJo software. Basophil cells were identified as CCR3+ cells. Activated basophils were defined as CCR3+CD63+ cells. The percentage (%) of activated basophils is displayed in the table 22 below.
Clones 13E4 and 18B9His induce basophil activation
Clone 18E2His2 does not induce basophil activation

TABLE 22
Basophil activation test

| | % of activated basophils |
|---|---|
| Irrelevant antibody | 3 |
| 18B9His-hIgG1-WT | 32 |
| 18B9His-hIgG1-ABDEG | 29 |
| 18E2His2-hIgG1-WT | 5 |
| 18E2His2-hIgG1-ABDEG | 4 |
| 13E4-hIgG1-WT | 18 |
| 13E4-hIgG1-ABDEG | 17 |

### Example 7. Clearance of IgE and IgG in Non-Disease Model by Anti-IgE-ABDEG™ Antibodies

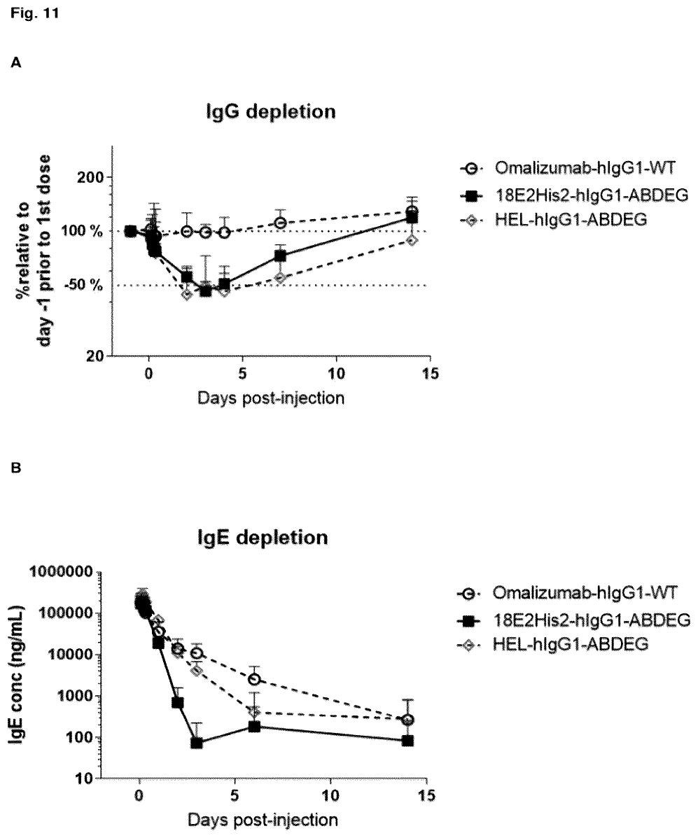

The ability of anti-hIgE-ABDEG™ antibodies to increase IgE and IgG clearance was analyzed in vivo in mice. rMota-hIgE was injected in C75BL6 mice 2h prior injection of anti-hIgE-ABDEG mAb. Blood was collected from mice and hIgE and murine IgG levels were measured by ELISA (see FIG. 11).
A non-IgE binding clone equipped with ABDEG™ mutation (HEL-hIgG1-ABDEG) induced IgG but not IgE depletion
Omalizumab was unable to induce IgE or IgG depletion
The clone 18E2His2-hIgG1-ABDEG induced IgG depletion and IgE depletion.

### Example 8 Germlining of Llama Anti-IgE Fab Clones

Selected anti-IgE Fab clones from Examples 2 and 3 were subjected to germlining by grafting the llama CDR sequences into human framework sequences. The Fab clones that were germlined were: 13E4; 18E2_VH_35H_VL_Y34H (18E2His2); VL18E2_Y34H; VH18E2_S35H; and VL18B9_Y49H (18B9His). The VH and VL sequences of the germlined clones are shown in Table 23 below.

TABLE 23
VH and VL sequences of the germlined anti-IgE Fab clones

| | | SEQ | | SEQ |
|---|---|---|---|---|
| Antibody clone | VH | ID NO. | VL | ID NO. |
| 13E4_MG | EVQLLESGGGLVQPGGSL | 171 | SSELTQDPAVSVALGQTV | 172 |
| | RLSCAASGFTFSSYVMSW | | RITCQGGSLGSNYAYWYQ | |
| | VRQAPGKGLEWVSSIYHD | | QKPGQAPVLVIYDDDSRP | |
| | GSHTYYADFVKGRFTISR | | SGIPDRFSGSSSGNTASL | |
| | DNSKNTLYLQMNSLRAED | | TITGAQAEDEADYYCQSA | |
| | TAVYYCAKGTSYSGSYYY | | DSNGNAVFGGGTQLTVL | |
| | TDPFFGSWGQGTLVTVSS | | | |
| 18E2His2_MG | EVQLLESGGGLVQPGGSL | 173 | SSELTQDPAVSVALGQTV | 174 |
| | RLSCAASGFTFSSYVMHW | | RITCQGDRLGSRYIHWYQ | |
| | VRQAPGKGLEWVSSIYHD | | QKPGQAPVLVIYDDDRRP | |
| | GSHTYYADFVKGRFTISR | | SGIPDRFSGSSSGNTASL | |
| | DNSKNTLYLQMNSLRAED | | TITGAQAEDEADYYCQSA | |
| | TAVYYCAKGTSYSGSYYY | | DSSGNPVFGGGTQLTVL | |
| | TDPFFGSWGQGTLVTVSS | | | |
| 18E2_VL | QLLESGGGLVQPGGSLRL | 215 | SSELTQDPAVSVALGQTV | 174 |
| Y34H_MG | SCAASGFTFSSYVMSWVR | | RITCQGDRLGSRYIHWYQ | |
| | QAPGKGLEWVSSIYHDGS | | QKPGQAPVLVIYDDDRRP | |
| | HTYYADFVKGRFTISRDN | | SGIPDRFSGSSSGNTASL | |
| | SKNTLYLQMNSLRAEDTA | | TITGAQAEDEADYYCQSA | |
| | VYYCAKGTSYSGSYYYTD | | DSSGNPVFGGGTQLTVL | |
| | PFFGSWGQGTLVTVSS | | | |
| 18E2_H | EVQLLESGGGLVQPGGSL | 173 | SSELTQDPAVSVALGQTV | 216 |
| S35H_MG | RLSCAASGFTFSSYVMHW | | RITCQGDRLGSRYIYWYQ | |
| | VRQAPGKGLEWVSSIYHD | | QKPGQAPVLVIYDDDRRP | |
| | GSHTYYADFVKGRFTISR | | SGIPDRFSGSSSGNTASL | |
| | DNSKNTLYLQMNSLRAED | | TITGAQAEDEADYYCQSA | |
| | TAVYYCAKGTSYSGSYYY | | DSSGNPVFGGGTQLTVL | |
| | TDPFFGSWGQGTLVTVSS | | | |
| 18B9His_MG | EVQLLESGGGLVQPGGSL | 175 | SSELTQDPAVSVALGQTV | 176 |
| | RLSCAASGFTFSSYVMSW | | RITCQGGSLGSSYVHWYQ | |
| | VRQAPGKGLEWVSSIYSD | | QKPGQAPVLVIYDGDSRP | |
| | GSHTYYADSVKGRFTISR | | SGIPDRFSGSSSGNTASL | |
| | DNSKNTLYLQMNSLRAED | | TITGAQAEDEADYYCQSA | |
| | TAVYYCAKNLEHYSGSYY | | DSSGNAVFGGGTQLTVL | |
| | YTDPRYDYWGQGTLVTVS | | | |
| | S | | | |

MG denotes germlined variant

### Competition ELISA

In the exact set-up to that used during the initial screening, the inhibition of hIgE binding to hFcαRIα by the 5 different anti-hIgE germlined clones was analyzed by ELISA at pH 7.4 and pH 6. The raw data (OD values) were plotted on GraphPad Prism 7.01. The IC50 values of each antibody were calculated with a non-linear regression (log(agonist) vs. response Variable slope (four parameters)). The results are shown in Table 24 below. All clones were still able to bind to IgE. As previously observed, clone 13E4 showed the highest affinity to hIgE at pH 7.4. The clone 18E2VHS35HVLY34HMG (germlined 18E2His2) showed the highest pH-dependent differential affinity to hIgE.

TABLE 24
IC50 (ng/ml) of inhibition of hIgE binding to FcεRI by ELISA

| | IC50 (ng/ml) | IC50 (ng/ml) |
|---|---|---|
| Germlined clone | (pH 7.4) | (pH 6) |
| 13E4_MG | 24.21 | 63.31 |
| 18E2_VHS35H_VLY34H_MG | 3911 | ~11280286 |
| 18E2_VLY34H_MG | 705.6 | 61380 |
| 18E2_VHS35H_MG | 65.68 | 671.3 |
| VL18B9_Y49H_MG | 176.5 | 1739 |

MG denotes germlined variant

### Example 9 Engineering PH-Dependent Variants of the Anti-IgE Antibody CL-2C

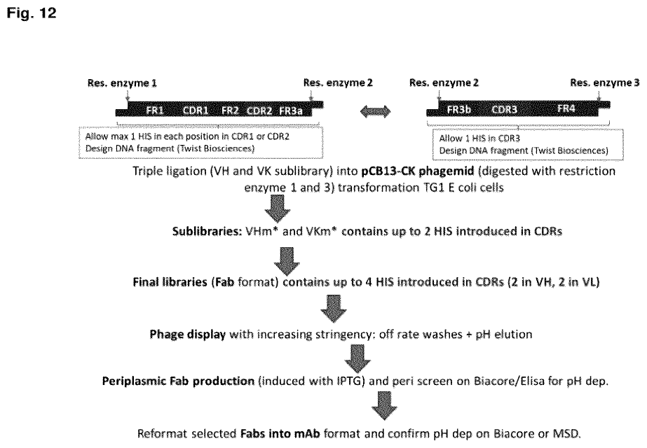

pH-dependent variants of the anti-IgE Fab of clone CL-2C were engineered according to the method depicted schematically in FIG. 12. The different stages of the method are described in more detail below.

### A. Design of Gene Fragments

Protein sequences for the VH and VL (Vκ) domains of clone CL-2C are described in US Patent U.S. Pat. No. 7,531,169, incorporated herein by reference. Starting from these VH and VL domains, histidine mutations were introduced at each position in the CDR regions (VH and VL) according to the approach depicted in the first step of the schematic shown in FIG. 12. The Kabat numbering scheme was used to number the amino acid residues of the variable domains. Gene fragments were designed with the desired mutations in the CDRs of the Vκ and VH variable regions together with suitable cloning sites. Framework region 3 (FR3) was divided into FR3a and FR3b with a cloning site in-between (as shown in FIG. 12).

### B. Library Construction

As a first step towards library construction, a Vκ mutant (Vκm) sub-library was constructed by cloning of the BsmBI-digested Vκ gene fragments into the ApaLI/XhoI pCB13-CK phagemid vector. Starting from this Vκ sub-library, final Fab libraries were generated using two approaches. (A and B explained below)

In accordance with approach A, triple ligation was carried out by cloning of the two SfiI/BsmBI(NheI compatible)-digested mutant VH (VHm) gene fragments into the SfiI/NheI-digested Vκm sub-library (contains the hCH1). In accordance with approach B, ligation was carried out by combined cloning of the SfiI/BsmBI-digested VHm-gene fragments with hCH1 NheI/NotI extracted from pCB13-CK into the SfiI/NotI-digested Vκm sub-library through a 4 points ligation. Ligation was followed by transformation into TG1 E. coli electrocompetent cells. Final Fab libraries contained up to 4 His mutations in the CDRs (0-1 in HCDR1 or HCDR2 and 0-1 in HCDR3, 0-1 in LCDR1 or LCDR2, 0-1 in LCDR3). Sequence analysis was performed on 32 random clones out of the final Vκ (using M13R) and VH libraries (using PelB3) from both approaches A and B. Ligation was followed by transformation into TG1 E. coli electrocompetent cells. VHm or Vκm sub-libraries contained up to 2 His mutations (0-1 His in CDR1 or CDR2 and 0-1 His in CDR3). Control Fabs were generated by separate cloning of 1Vκlig01A (WT Vκ) and 1VHlig01A (WT VH). From approach A, 25 out of 32 Vκ sequenced clones (78%) showed correct V-Regions sequences. From approach B, this was the case for 24 out of the 32 clones (75%). From approach A, 17 out of 32 VH sequenced clones (53%) showed correct V-Regions sequences. From approach B, this was the case for 16 out of the 32 clones (50%).

### C. Selection

Fab phage display was performed using the Fab libraries from both approaches and selection was performed with increased stringency, combining off-rate washing (washing in the presence of soluble target) and pH elution. Eluted phages were used for infection of E. coli TG1 cells. Output of eluted phages from several selection rounds were plated to obtain single colonies. Individual clones were picked at random and six master plates were generated.

### D. Screening by IgE Binding ELISA

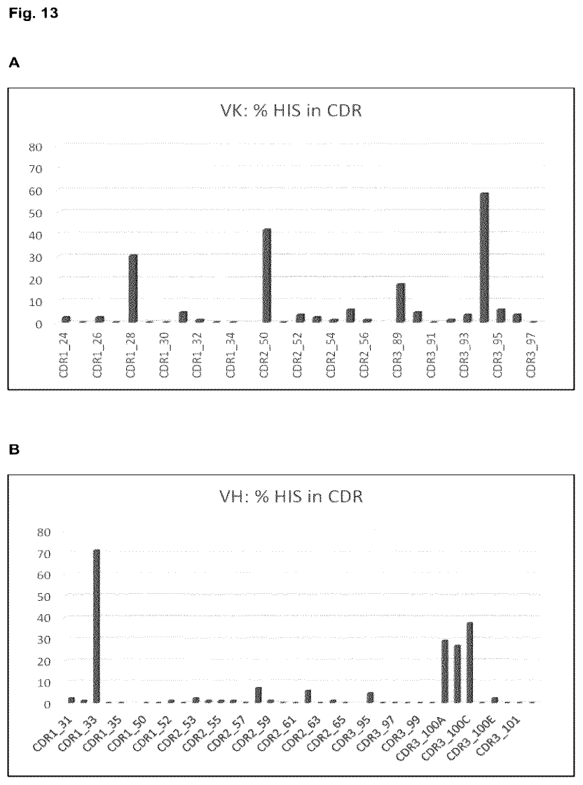

Periplasmic extracts (crude fraction containing the secreted monomeric Fabs called PERI) were produced from 1 ml E. coli cultures (induced with IPTG) derived from all generated master plates. A hIgE binding ELISA was carried out precisely in accordance with the protocol described above in Example 2. Sequencing of clones exhibiting pH-dependent binding to hIgE revealed positions in Vκ and VH enriched in His mutations. These results are depicted schematically in FIG. 13.

### E. Reformatting

The 8 Vκ and 5 VH strings shown in Table 25 below were re-cloned into a mammalian expression vector containing the human constant domain (human hIgG1) for further characterization.

TABLE 25

| | Selected V_{K} | Selected VH |
|---|---|---|
| HIS | VK28H94H | VH33H100CH |
| position | VK50H92D89H | VH33H100BH |
| | VK50H94H | VH33H100AH |
| | VK50H92D94H | VH58H100BH |
| | VK50H | |
| | VK94H | |
| | VKWT | VHWT |

DNA String fragments were designed and ordered from Geneart for each VH and VL and subsequently digested with restriction enzymes (BsmBi). After digestion and clean-up, ligation of the DNA (VH or VL) was performed into BsmBi pre-digested vectors containing the constant domains of the human kappa light chain (pUPEX116.08 for Vκ) or of the human IgG1-ABDEG™ heavy chain (CH1-CH2-CH3, pUPEX32a). The ligated products were transformed into Top10 bacteria by heat shock and transferred onto agarose plates with Ampicillin (resistance gene of the vectors). For each clone (HC and LC), four to eight colonies were picked and sent for sequencing. The clones that showed the proper insert were selected and amplified in order to purify the DNA sequence (by MidiPrep).

Production of 35 human IgG1 antibodies, resulting from combination of all Vκs with all VHs, was carried out by transfection of HEK293E cells (using the Polyethylenimine (PEI) with a mix containing the heavy and light chain DNA expression vectors in a 1/1 ratio). After allowing cells to express protein for 6 days, human monoclonal antibodies were purified from the cell supernatant using protein-A sepharose beads. Finally, SDS-PAGE analysis was carried out to assess the purity and the integrity of the antibodies.

### F. Characterization of PH-Engineered Anti-hIgE Antibodies

The hIgE binding properties of the engineered CL-2C antibody panel were assessed by SPR analysis (with a Biacore 3000) and by IgE binding ELISA, in accordance with the protocols described in Example 2.

i. SPR Analysis

The results of the SPR analysis are shown in Table 26 below. The ratio of K_{D} measured at pH 5.5 versus K_{D} measured at pH 7.4 was calculated for each of the CL-2C antibodies.

TABLE 26
Binding of pH-engineered
CL-2C antibodies to hIgE as measured by Biacore

| | | | KD Ratio |
|---|---|---|---|
| Antibody | V_{K} | VH | pH 5.5/pH 7.4 |
| CL-20 mAb 1 | V_{K}28H94H | VH58H100BH | 4.6 |
| CL-20 mAb 2 | | VH33H100CH | 8.5 |
| CL-2C mAb 3 | | VH33H100BH | 8.4 |
| CL-2C mAb 4 | | VH33H100AH | 3.3 |
| CL-2C mAb 5 | | VHWT | 0.3 |
| CL-2C mAb 6 | V_{K}50H92D89H | VH58H100BH | 6.1 |
| CL-2C mAb 7 | | VH33H100CH | 7.9 |
| CL-2C mAb 8 | | VH33H100BH | 2.8 |
| CL-2C mAb 9 | | VH33H100AH | 2.5 |
| CL-2C mAb 10 | | VHWT | 0.7 |
| CL-2C mAb 11 | V_{K}50H94H | VH58H100BH | 6.0 |
| CL-2C mAb 12 | | VH33H100CH | 3.9 |
| CL-2C mAb 13 | | VH33H100BH | 17.3 |
| CL-2C mAb 14 | | VH33H100AH | 3.2 |
| CL-2C mAb 15 | | VHWT | 1.1 |
| CL-2C mAb 16 | V_{K}50H92D94H | VH58H100BH | 4.7 |
| CL-2C mAb 17 | | VH33H100CH | 7.1 |
| CL-2C mAb 18 | | VH33H100BH | 0.2 |
| CL-2C mAb 19 | | VH33H100AH | 5.2 |
| CL-2C mAb 20 | | VHWT | 1.1 |
| CL-2C mAb 26 | V_{K}50H | VH58H100BH | 8.9 |
| CL-2C mAb 27 | | VH33H100CH | 7.4 |
| CL-2C mAb 28 | | VH33H100BH | 9.0 |
| CL-2C mAb 29 | | VH33H100AH | 3.8 |
| CL-2C mAb 30 | | VHWT | 1.0 |
| CL-2C mAb 31 | V_{K}94H | VH58H100BH | 5.5 |
| CL-2C mAb 32 | | VH33H100CH | 6.2 |
| CL-2C mAb 33 | | VH33H100BH | 7.0 |
| CL-2C mAb 34 | | VH33H100AH | 3.0 |
| CL-2C mAb 35 | | VHWT | 0.6 |
| CL-2C mAb 36 | V_{K}WT | VH58H100BH | 4.3 |
| CL-2C mAb 37 | | VH33H100CH | 11.1 |
| CL-2C mAb 38 | | VH33H100BH | 6.5 |
| CL-2C mAb 39 | | VH33H100AH | 4.6 |
| CL-2C mAb 40 | | VHWT | 0.6 |

Not all clones were found to be pH-dependent. By SPR analysis, CL-2C mAb3 showed the PGP-466.4M highest pH-dependency.

ii. IgE Binding ELISA

The results of the hIgE binding ELISA are shown in Table 27 below. The ratio of 00450 measured at pH 7.4 versus 00450 measured at pH 6 was calculated for each of the CL-2C antibodies.

TABLE 27
Binding of pH-engineered CL-2C
antibodies to hIgE as measured by ELISA

| | | | Ratio OD450 |
|---|---|---|---|
| Antibody | V_{K} | VH | pH 7.4/pH 6 |
| CL-2C mAb 1 | VK28H94H | VH58H100BH | 6.94 |
| CL-2C mAb 2 | | VH33H100CH | 7.11 |
| CL-2C mAb 3 | | VH33H100BH | 6.44 |
| CL-2C mAb 4 | | VH33H100AH | 5.26 |
| CL-2C mAb 5 | | VHWT | 0.73 |
| CL-2C mAb 6 | VK50H92D89H | VH58H100BH | 10.22 |
| CL-2C mAb 7 | | VH33H100CH | 1.43 |
| CL-2C mAb 8 | | VH33H100BH | 1.12 |
| CL-2C mAb 9 | | VH33H100AH | 1.45 |
| CL-2C mAb 10 | | VHWT | 2.77 |
| CL-2C mAb 11 | VK50H94H | VH58H100BH | 5.90 |
| CL-2C mAb 12 | | VH33H100CH | 1.54 |
| CL-2C mAb 13 | | VH33H100BH | 1.28 |
| CL-2C mAb 14 | | VH33H100AH | 1.53 |
| CL-2C mAb 15 | | VHWT | 4.07 |
| CL-2C mAb 16 | VK50H92D94H | VH58H100BH | 2.24 |
| CL-2C mAb 17 | | VH33H100CH | 1.03 |
| CL-2C mAb 18 | | VH33H100BH | 0.97 |
| CL-2C mAb 19 | | VH33H100AH | 1.04 |
| CL-2C mAb 20 | | VHWT | 5.46 |
| CL-2C mAb 26 | VK50H | VH58H100BH | 44.56 |
| CL-2C mAb 27 | | VH33H100CH | 19.40 |
| CL-2C mAb 28 | | VH33H100BH | 5.54 |
| CL-2C mAb 29 | | VH33H100AH | 15.28 |
| CL-2C mAb 30 | | VHWT | 13.97 |
| CL-2C mAb 31 | VK94H | VH58H100BH | 10.94 |
| CL-2C mAb 32 | | VH33H100CH | 4.13 |
| CL-2C mAb 33 | | VH33H100BH | 2.61 |
| CL-2C mAb 34 | | VH33H100AH | 5.50 |
| CL-2C mAb 35 | | VHWT | 0.69 |
| CL-2C mAb 36 | VKWT | VH58H100BH | 10.71 |
| CL-2C mAb 37 | | VH33H100CH | 10.34 |
| CL-2C mAb 38 | | VH33H100BH | 4.48 |
| CL-2C mAb 39 | | VH33H100AH | 7.59 |
| CL-2C mAb 40 | | VHWT | 1.00 |

Similar to the Biacore results, not all clones were found to be pH-dependent. By ELISA analysis, CL-2C mAb26 showed the highest pH-dependency.

The CDR, VH domain and VL domain sequences of the pH-engineered CL-2C antibody variants are shown in Tables 28, 29 and 30 below.

TABLE 28
Heavy chain CDR sequences of pH-engineered VH domains

| Antibody VH | | SEQ ID | | SEQ ID | | SEQ ID |
|---|---|---|---|---|---|---|
| domains | CDR1 | NO. | CDR2 | NO. | CDR3 | NO. |
| VH58H100BH | WYWLE | 151 | EIDPGTFTTHYNEKFKA | 177 | FSHFSGSHYDYFDY | 178 |
| VH33H100CH | WYHLE | 179 | EIDPGTFTTNYNEKFKA | 152 | FSHFSGSNHDYFDY | 180 |
| VH33H100BH | WYHLE | 179 | EIDPGTFTTNYNEKFKA | 152 | FSHFSGSHYDYFDY | 178 |
| VH33H100AH | WYHLE | 179 | EIDPGTFTTNYNEKFKA | 152 | FSHFSGHNYDYFDY | 181 |

TABLE 29
Light chain CDR sequences of pH-engineered VL domains

| | | SEQ ID | | SEQ ID | | SEQ ID |
|---|---|---|---|---|---|---|
| Antibody | CDR1 | NO. | CDR2 | NO. | CDR3 | NO. |
| VK28H94H | RASQHIGTNIH | 182 | YASESIS | 156 | QQSWSHPTT | 183 |
| VK50H92D89H | RASQSIGTNIH | 155 | HASESIS | 184 | HQSDSWPTT | 214 |
| VK5OH94H | RASQSIGTNIH | 155 | HASESIS | 184 | QQSWSHPTT | 183 |
| VK50H92D94H | RASQSIGTNIH | 155 | HASESIS | 184 | QQSDSHPTT | 213 |
| VK50H | RASQSIGTNIH | 155 | HASESIS | 184 | QQSWSWPTT | 183 |
| VK94H | RASQSIGTNIH | 155 | YASESIS | 156 | QQSWSHPTT | 183 |

TABLE 30
VH and VL domain sequences of pH-engineered antibodies

| | | SEQ | | | SEQ |
|---|---|---|---|---|---|
| | | ID | | | ID |
| VH | Sequence | NO. | VL | Sequence | NO. |
| VH58H | QVQLVQSGAEVMKPGSSVKVSCKASGYT | 185 | VK28H | EIVMTQSPATLSVSPGERATLSCRASQHIGTNIHWYQQKP | 189 |
| 100BH | FSWYWLEWVRQAPGHGLEWMGEIDPGT | | 94H | GQAPRLLIYYASESISGIPARFSGSGSGTEFTLTISSLQSE | |
| | FTTHYNEKFKARVTFTADTSTSTAYMELS | | | DFAVYYCQQSWSHPTTFGGGTKVEIK | |
| | SLRSEDTAVYYCARFSHFSGSHYDYFDY | | | | |
| | WGQGTLVTVSS | | | | |
| VH33H | QVQLVQSGAEVMKPGSSVKVSCKASGYT | 186 | VK50H | EIVMTQSPATLSVSPGERATLSCRASQSIGTNIHWYQQKP | 190 |
| 100CH | FSWYHLEWVRQAPGHGLEWMGEIDPGT | | 92D89H | GQAPRLLIYHASESISGIPARFSGSGSGTEFTLTISSLQSE | |
| | FTTNYNEKFKARVTFTADTSTSTAYMELS | | | DFAVYYCHQSDSWPTTFGGGTKVEIK | |
| | SLRSEDTAVYYCARFSHFSGSNHDYFDY | | | | |
| | WGQGTLVTVSS | | | | |
| VH33H | QVQLVQSGAEVMKPGSSVKVSCKASGYT | 187 | VK50H | EIVMTQSPATLSVSPGERATLSCRASQSIGTNIHWYQQKP | 191 |
| 100BH | FSWYHLEWVRQAPGHGLEWMGEIDPGT | | 94H | GQAPRLLIYHASESISGIPARFSGSGSGTEFTLTISSLQSE | |
| | FTTNYNEKFKARVTFTADTSTSTAYMELS | | | DFAVYYCQQSWSHPTTFGGGTKVEIK | |
| | SLRSEDTAVYYCARFSHFSGSHYDYFDY | | | | |
| | WGQGTLVTVSS | | | | |
| VH33H | QVQLVQSGAEVMKPGSSVKVSCKASGYT | 188 | VK50H | EIVMTQSPATLSVSPGERATLSCRASQSIGTNIHWYQQKP | 192 |
| 100AH | FSWYHLEWVRQAPGHGLEWMGEIDPGT | | 92D94H | GQAPRLLIYHASESISGIPARFSGSGSGTEFTLTISSLQSE | |
| | FTTNYNEKFKARVTFTADTSTSTAYMELS | | | DFAVYYCQQSDSHPTTFGGGTKVEIK | |
| | SLRSEDTAVYYCARFSHFSGHNYDYFDY | | | | |
| | WGQGTLVTVSS | | | | |
| | | | VK50H | EIVMTQSPATLSVSPGERATLSCRASQSIGTNIHWYQQKP | |
| | | | | GQAPRLLIYHASESISGIPARFSGSGSGTEFTLTISSLQSE | 193 |
| | | | | DFAVYYCQQSWSWPTTFGGGTKVEIK | |
| | | | VK94H | EIVMTQSPATLSVSPGERATLSCRASQSIGTNIHWYQQKP | 194 |
| | | | | GQAPRLLIYYASESISGIPARFSGSGSGTEFTLTISSLQSE | |
| | | | | DFAVYYCQQSWSHPTTFGGGTKVEIK | |

### Example 10 Engineering PH-Dependent Variants of the Anti-IgE Antibody Omalizumab

The omalizumab antibody was subjected to a process of affinity maturation prior to the generation of pH-dependent variants. These methods are described in detail below.

### Omalizumab Affinity Maturation

### A. Library Generation

Key residues in the CDR regions of the omalizumab VH and VL domains were subjected to mutagenesis. The numbering of the CDR amino acid residues was performed according to the Kabat numbering scheme. A maximum of six residues were mutated per CDR sub-library (giving a theoretical diversity of 6.40×10⁷. Library design was based on solvent exposed residues and/or high variability based on natural antibody sequences.

### B. Generation of Parental Fab

2 μg omalizumab VHWT cDNA was digested with NcoI/NheI and 2 μg of omalizumab VkWT was digested with ApalI/BswiI. Samples were separated in a 1% agarose gel and purified for further ligation with pCB13.ck4. 43 ng of omalizumab VHWT and omalizumab VkWT DNA fragments were ligated with 200 ng of pCB 13 Ck4 vector digested with NcoI/NheI and ApalI/BsiWI, respectively. Transformation of purified 10 μl ligation was performed with 25 μl ECC TG1 cells (Lucigen Cat nr 60502 2).

### C. Variant Fab Library Generation

Vk and VH gene variants were generated via a PCR and gene assembly protocol using eight overlapping oligonucleotides. Libraries were generated by ligation of NcoI/NheI-digested VH into NcoI/NheI-digested VLWT pCB13 and ApalI/BsiWI-digested VL into ApalI BsiWI-digested VHWTpCB13. Libraries were transformed into ECC TG1 cells (Lucigen Cat nr 60502 2).

### D. Selection

Fab phage display was performed using the Fab libraries generated as described above. Selection was carried out with increased stringency and off-rate washing (washing in the presence of soluble target). Eluted phages were used for infection of E. coli TG1 cells. Output of eluted phages from several selection rounds were plated to obtain single colonies. Individual clones were picked at random into master plates (MP).

E. Screening for hIgE Binding

Periplasmic extracts (crude fraction containing the secreted monomeric Fabs called PERI) were produced from 1 ml E. coli cultures (induced with IPTG) derived from all generated master plates. The binding of Fab periplasmic extract to hIgE was assessed by SPR analysis, as described in Example 2. The results are shown in Table 31 below.

TABLE 31
Binding of affinity-matured omalizumab antibodies to hIgE as measured by
Biacore

| | | Native | | Recombinant IgE | | | | |
|---|---|---|---|---|---|---|---|---|
| | | IgE (Abcam) | | (argenx) | | Off-rate improvement ratio | | |
| | | pH 7.4 | | pH 7.4 | | IgE | IgE | |
| VH ID | VL ID | kd (1/s) | R0 (RU) | kd (1/s) | R0 (RU) | (abcam) | (argenx) | Average |
| VH15 | VL3 | 3.50E−04 | 235.19 | 2.69E−04 | 284.68 | 14.83 | 18.03 | 16.43 |
| VH11 | VL3 | 4.12E−04 | 190.02 | 3.21E−04 | 231.8 | 12.6 | 15.11 | 13.85 |
| VH14 | VL3 | 4.75E−04 | 235.86 | 3.66E−04 | 290.2 | 10.93 | 13.25 | 12.09 |
| VH13 | VL3 | 4.46E−04 | 235.08 | 3.45E−04 | 290.48 | 11.64 | 14.06 | 12.85 |
| VH17 | VL3 | 7.99E−04 | 262.74 | 6.16E−04 | 321.98 | 6.5 | 7.87 | 7.18 |
| VH18 | VL3 | 6.47E−04 | 227.47 | 5.34E−04 | 274.71 | 8.02 | 9.08 | 8.55 |
| VH16 | VL3 | 6.79E−04 | 261.73 | 5.26E−04 | 317.15 | 7.64 | 9.22 | 8.43 |
| VH7 | VLWT | 1.46E−03 | 253.32 | 6.78E−04 | 260.04 | 3.55 | 7.15 | 5.35 |
| VH5 | VL4 | 2.14E−03 | 212.18 | 1.49E−03 | 268.21 | 2.43 | 3.26 | 2.84 |
| VH15 | VLWT | 2.01E−03 | 217.55 | 1.57E−03 | 268.12 | 2.58 | 3.09 | 2.84 |
| VH3 | VL3 | 4.81E−04 | 193.93 | 3.52E−04 | 229.27 | 10.79 | 13.78 | 12.28 |
| VH13 | VLWT | 2.47E−03 | 208.11 | 1.97E−03 | 255.78 | 2.1 | 2.46 | 2.28 |
| VH5 | VL3 | 5.58E−04 | 236.53 | 4.32E−04 | 284.16 | 9.3 | 11.23 | 10.26 |
| VH14 | VLWT | 2.64E−03 | 224.11 | 2.16E−03 | 272.85 | 1.97 | 2.25 | 2.11 |
| VHWT | VLWT | 5.19E−03 | 186.94 | 4.85E−03 | 204.67 | 1 | 1 | 1 |

One particular clone, VH15VL3, showed the highest affinity increase and was selected for further pH engineering.

### Omalizumab pH Engineering

A. Production of pH-Dependent Omalizumab Antibody Variants

In a similar approach to that described in Example 9 for CL-2C, histidine mutations were introduced in each position in the CDR regions of the VH and VL domains of the omalizumab parental antibody.

Using this approach, 2 mutations in the VH domain and 2 mutations in the VL domain were chosen based on their enrichment in pH-dependent clones:
VH: G55H in CDR2 and W100bH in CDR3
Vκ: D28H in CDR1 and S31H in CDR1

The identified histidine “hotspots” noted above were inserted into the affinity-matured variant of omalizumab—VH15VL3. The production of 16 human IgG1 antibodies, resulting from the combination of all Vκs with all VHs, was carried out as previously described.

### B. Characterization of PH-Engineered Omalizumab Antibody Variants

The hIgE binding properties of the engineered omalizumab antibody panel were assessed by SPR analysis (with a Biacore 3000), IgE binding ELISA and by IgE competition ELISA, according to the protocols described in Example 2.

i. SPR Analysis

The results of the SPR analysis are shown in Table 32 below.

TABLE 32
Binding of pH-engineered omalizumab antibodies to hIgE as measured by Biacore

| | | pH 6.0 | | | pH 7.4 | | | Ratio |
|---|---|---|---|---|---|---|---|---|
| | | | | KD | | | | pH |
| Antibody | | ka (1/Ms) | kd (s) | (nM) | ka (1/Ms) | kd (s) | KD (nM) | 7.4/pH 6.0 |
| OMA VH15 | VL3 | 2.81E+05 | 2.90E−04 | 1.03 | 1.30E+05 | 1.14E−08 | 0.0000874 | |
| | VL3D25H | 2.81E+05 | 7.36E−04 | 2.62 | 2.64E+05 | 4.41E−05 | 0.1675 | 15.6 |
| | VL3S31H | 2.61E+05 | 2.28E−03 | 8.93 | 3.10E+05 | 1.66E−04 | 0.534 | 16.7 |
| | VL3D28HS31H | 3.10E+05 | 3.98E−03 | 12.85 | 3.02E+05 | 3.34E−04 | 1.1 | 11.7 |
| OmaVH15G55H | VL3 | 2.91E+05 | 5.24E−04 | 1.805 | 3.23E+05 | 1.05E−04 | 0.3255 | 5.6 |
| | VL3D25H | 2.65E+05 | 1.17E−03 | 4.395 | 2.83E+05 | 2.06E−04 | 0.7275 | 6.0 |
| | VL3S31H | 3.29E+05 | 2.97E−03 | 9.07 | 3.01E+05 | 4.02E−04 | 1.335 | 6.8 |
| | VL3D28HS31H | 3.26E+05 | 5.27E−03 | 16.05 | 2.86E+05 | 7.22E−04 | 2.52 | 6.4 |
| Oma VH15W100b | VL3 | 2.94E+05 | 8.75E−03 | 29.75 | 3.51E+05 | 1.03E−03 | 2.93 | 10.2 |
| | VL3D25H | 2.32E+05 | 1.48E−02 | 64.45 | 2.13E+05 | 1.82E−03 | 8.59 | 7.5 |
| | VL3S31H | 2.06E+05 | 3.75E−02 | 190.5 | 3.98E+05 | 2.44E−03 | 6.125 | 31.1 |
| | VL3D28HS31H | 4.53E+05 | 8.95E−02 | 206 | 2.90E+05 | 2.51E−03 | 8.78 | 23.5 |
| OmaVH15G55HW100b | VL3 | 1.70E+05 | 2.54E−02 | 149.5 | 2.61E+05 | 2.26E−03 | 8.685 | 17.2 |
| | VL3D25H | 1.23E+05 | 6.48E−02 | 136.5 | 2.00E+05 | 3.03E−03 | 15.2 | 9.0 |
| | VL3S31H | 2.43E+05 | 9.86E−02 | 312 | 2.53E+05 | 3.91E−03 | 15.4 | 20.3 |
| | VL3D28HS31H | 0.00E+00 | 0.00E+00 | 0 | 1.09E+03 | 3.56E−03 | | |
| Control | 18E2VLHis_MG | 4.30E+05 | 6.15E−02 | 205 | 3.92E+05 | 2.23E−03 | 5.91 | 34.7 |
| | hIgG1-ABDEG | | | | | | | |
| | Omalizumab | 3.28E+05 | 3.54E−03 | 10.825 | 3.03E+05 | 1.35E−03 | 4.445 | 2.4 |
| | hIgG1-WT | | | | | | | |

The results revealed that not all clones are pH-dependent. OmaVH15W100b-VL3S31H showed the highest pH-dependency as measured by Biacore.

ii. IgE Binding ELISA

The results of the hIgE binding ELISA are shown in Table 33 below.

TABLE 33
Binding of pH-engineered
omalizumab antibodies to hIgE as measured by ELISA

| | | EC50 (ng/ml) | | Ratio EC50 | Ratio EC50 |
|---|---|---|---|---|---|
| Antibody | | pH 7.4 | pH 5.5 | pH 5.5/7.4 | pH 7.4/5.5 |
| OMA VH15 | VL3 | 20.2 | 21.74 | 1.1 | 0.93 |
| | VL3D28H | 16.28 | 16.42 | 1.0 | 0.99 |
| | VL3S31H | 20.04 | 17.86 | 0.9 | 1.12 |
| | VL3D28HS31H | 15.34 | 17.77 | 1.2 | 0.86 |
| Omalizumab | | 29.55 | 33.52 | 1.1 | 0.88 |
| OmaVH15G55H | VL3 | 25.51 | 22.02 | 0.9 | 1.16 |
| | VL3D28H | 26.43 | 25.07 | 0.9 | 1.05 |
| | VL3S31H | 15.3 | 22.96 | 1.5 | 0.67 |
| | VL3D28HS31H | 15.41 | 22.96 | 1.5 | 0.67 |
| Omalizumab | | 29.24 | 40.71 | 1.4 | 0.72 |
| 18E2VL | | 19.96 | 32.25 | 1.6 | 0.62 |
| Oma VH15W100b | VL3 | 17.42 | 32.65 | 1.9 | 0.53 |
| | VL3D28H | 15.04 | / | | |
| | VL3S31H | 15.25 | / | | |
| | VL3D28HS31H | 15.61 | / | | |
| Omalizumab | | 24.39 | 34.12 | 1.4 | 0.71 |
| CL-2C mAb37 | | 14.85 | 37.26 | 2.5 | 0.40 |
| OmaVH15G55HW100b | VL3 | 16.41 | 221 | 13.5 | 0.07 |
| | VL3D28H | 13.53 | 130 | 9.6 | 0.10 |
| | VL3S31H | 19.13 | / | | |
| | VL3D28HS31H | / | / | | |

| | | | | |
|---|---|---|---|---|
| 18E2VL | 16.34 | 26.88 | 1.6 | 0.61 |

Similar to the results seen with the SPR analysis, not all clones were pH-dependent. OmaVH15W100b-VL3D28H, OmaVH15W100b-VL3S31H, OmaVH15W100b-VL3D28HS31H, OmaVH15G55hW100b-VL3S31H and OmaVH15G55hW100b-VL3D28HS31H showed the highest pH dependency as measured by hIgE binding ELISA.

iii. IgE Competition ELISA

The results of the hIgE competition ELISA are shown in Table 34 below.

TABLE 34
Activity of pH-engineered omalizumab
antibodies to hIgE as measured by competition ELISA

| | | IC50 (nM) |
|---|---|---|
| OMA VH15 | VL3 | 0.08025 |
| | VL3D28H | 0.1366 |
| | VL3S31H | 0.2063 |
| | VL3D28HS31H | 0.7218 |
| OmaVH15G55H | VL3 | 0.07529 |
| | VL3D28H | 0.1869 |
| | VL3S31H | 0.4848 |
| | VL3D28HS31H | 2.629 |
| Oma VH15W100b | VL3 | 1.402 |
| | VL3D28H | 35.05 |
| | VL3S31H | / |
| | VL3D28HS31H | 585.1 |
| OmaVH15G55HW100b | VL3 | 23.23 |
| | VL3D28H | 171.6 |
| | VL3S31H | 446.5 |
| | VL3D28HS31H | 9.487 |

| | |
|---|---|
| Omalizumab | 1.133 |
| 18E2VL | 6.057 |
| CL-2C mAb37 | 0.4834 |

In this experiment, OMAVH155VL3 showed the best affinity. Histidine engineering was found to affect the capacity for OMAVH15VL3 to inhibit IgE binding to FcεRIα.

The CDR, VH and VL sequences of clone VH15VL3 and the pH-engineered variants thereof are shown in Tables 35, 36 and 37 below.

TABLE 35
Heavy chain CDR sequences of engineered VH domains

| | | | | SEQ ID | | SEQ ID |
|---|---|---|---|---|---|---|
| Antibody VH domains | CDR1 | SEQ ID NO. | CDR2 | NO. | CDR3 | NO. |
| OMA VH15 | SGHRWE | 195 | SIHYDGSTNYNPSVKG | 196 | ATHYFGHWHFAV | 197 |
| OmaVH15G55H | SGHRWE | 195 | SIHYDHSTNYNPSVKG | 198 | ATHYFGHWHFAV | 197 |
| Oma VH15W100b | SGHRWE | 195 | SIHYDGSTNYNPSVKG | 196 | ATHYFGHHHFAV | 199 |
| OmaVH15G55HW100b | SGHRWE | 195 | SIHYDHSTNYNPSVKG | 198 | ATHYFGHHHFAV | 199 |

TABLE 36
Light chain CDR sequences of engineered VL domains

| | | | | SEQ | | SEQ |
|---|---|---|---|---|---|---|
| | | | | ID | CDR3 | ID |
| Antibody | CDR1 | SEQ ID NO. | CDR2 | NO. | | NO. |
| VL3 | RASQSVDYDGDSYMN | 147 | WGSYLRS | 200 | QQNAEDPYT | 201 |
| VL3D28H | RASQSVDYHGDSYMN | 202 | WGSYLRS | 200 | QQNAEDPYT | 201 |
| VL3S31H | RASQSVDYDGDHYMN | 203 | WGSYLRS | 200 | QQNAEDPYT | 201 |
| VL3D28HS31H | RASQSVDYHGDHYMN | 204 | WGSYLRS | 200 | QQNAEDPYT | 201 |

TABLE 37
VH and VL domain sequences of engineered omalizumab antibodies

| | | SEQ ID | | | SEQ ID |
|---|---|---|---|---|---|
| VH | Sequence | NO. | VL | Sequence | NO. |
| OMA VH15 | EVQLVESGGGLVQPGGSLRLS | 205 | OMA VL3 | DIQLTQSPSSLSASVGDRVTITCRAS | 209 |
| | CAVSGYSITSGHRWEWIRQAP | | | QSVDYDGDSYMNWYQQKPGKAPK | |
| | GKGLEWVASIHYDGSTNYNPS | | | LLIEWGSYLRSGVPSRFSGSGSGTD | |
| | VKGRITISRDDSKNTFYLQMNS | | | FTLTISSLQPEDFATYYCQQNAEDP | |
| | LRAEDTAVYYCARATHYFGHW | | | YTFGQGTKVEIK | |
| | HFAVWGQGTLVTVSS | | | | |
| OmaVH15G55H | EVQLVESGGGLVQPGGSLRLS | 206 | OmaVL3D28H | DIQLTQSPSSLSASVGDRVTITCRAS | |
| | CAVSGYSITSGHRWEWIRQAP | | | QSVDYHGDSYMNWYQQKPGKAPK | 210 |
| | GKGLEWVASIHYDHSTNYNPS | | | LLIEWGSYLRSGVPSRFSGSGSGTD | |
| | VKGRITISRDDSKNTFYLQMNS | | | FTLTISSLQPEDFATYYCQQNAEDP | |
| | LRAEDTAVYYCARATHYFGHW | | | YTFGQGTKVEIK | |
| | HFAVWGQGTLVTVSS | | | | |
| Oma | EVQLVESGGGLVQPGGSLRLS | 207 | OmaVL3S31H | DIQLTQSPSSLSASVGDRVTITCRAS | |
| VH15W100b | CAVSGYSITSGHRWEWIRQAP | | | QSVDYDGDHYMNWYQQKPGKAPK | 211 |
| | GKGLEWVASIHYDGSTNYNPS | | | LLIEWGSYLRSGVPSRFSGSGSGTD | |
| | VKGRITISRDDSKNTFYLQMNS | | | FTLTISSLQPEDFATYYCQQNAEDP | |
| | LRAEDTAVYYCARATHYFGHH | | | YTFGQGTKVEIK | |
| | HFAVWGQGTLVTVSS | | | | |
| OmaVH15G55H | EVQLVESGGGLVQPGGSLRLS | 208 | OmaVL3D28H | DIQLTQSPSSLSASVGDRVTITCRAS | 212 |
| W100b | CAVSGYSITSGHRWEWIRQAP | | S31H | QSVDYHGDHYMNWYQQKPGKAPK | |
| | GKGLEWVASIHYDHSTNYNPS | | | LLIEWGSYLRSGVPSRFSGSGSGTD | |
| | VKGRITISRDDSKNTFYLQMNS | | | FTLTISSLQPEDFATYYCQQNAEDP | |
| | LRAEDTAVYYCARATHYFGHH | | | YTFGQGTKVEIK | |
| | HFAVWGQGTLVTVSS | | | | |

### Example 11 Inhibition of IgE Binding to FcεRIα+ Cells

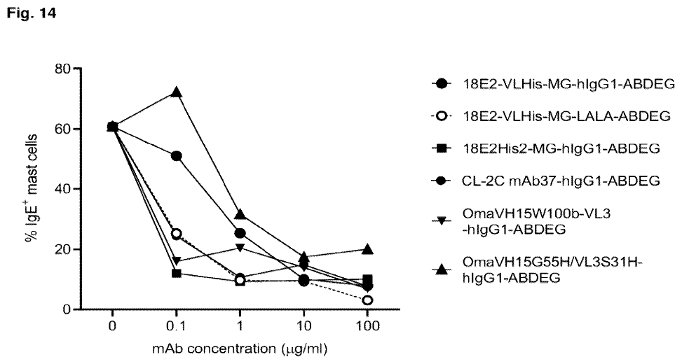

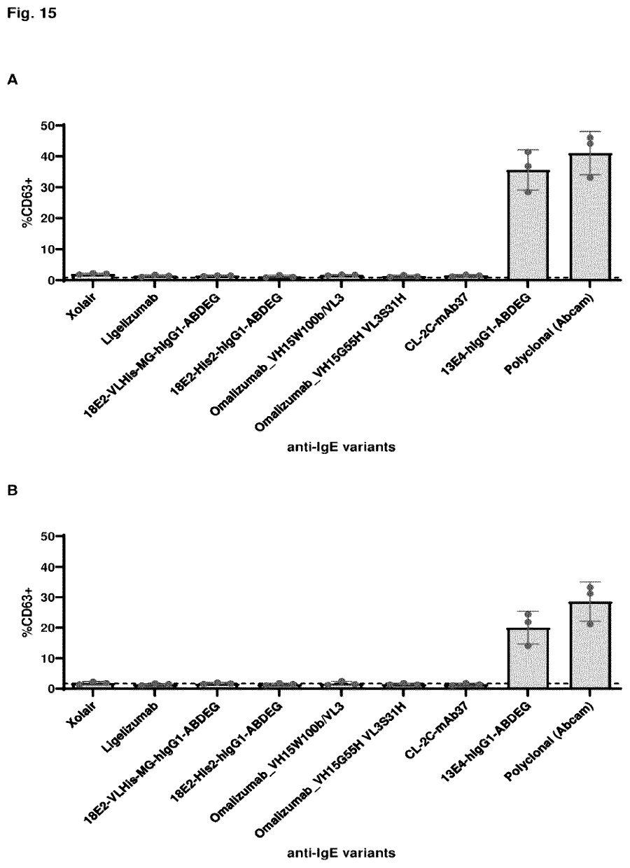

Human mast cells were cultured from CD34+ blood progenitors (healthy donor) with SCF, IL-6 and IL-3 for 12 weeks until they co-expressed KIT and FcεRI and were able to degranulate upon crosslinking of FcεRI. Chimeric NP-specific IgE (human constant region; mouse variable regions) was produced using JW8/5/13 cells (Sigma), and purified using omalizumab-coupled sepharose. NP-specific IgE was coupled to APC. Human mast cells were pre-incubated with various anti-IgE mAbs at a range of concentrations before addition of APC-hIgE. APC fluorescence was analysed after 1 h to determine the percentage (%) of IgE+ mast cells. The results are shown in FIG. 14. 18E2His2-MG-hIgG1-ABDEG showed the best competition capacity to human mast cells. OMAVH15G55H-VL3S31H showed the lowest competition capacity to human mast cells.

### Example 12 Safety Testing of Anti-IgE-ABDEG™ Antibodies

As described elsewhere herein, anti-IgE antibodies can exhibit undesirable properties such as the cross-linking of IgE already bound to FcεRIα at the cell surface. This cross-linking can lead to downstream effects such as mast cell and basophil activation, and initiate unwanted anaphylaxis. The ability of various anti-IgE antibodies described herein to bind to receptor-bound IgE and thus trigger downstream events was assessed as described below.

### A. Mast Cell Activation Assay

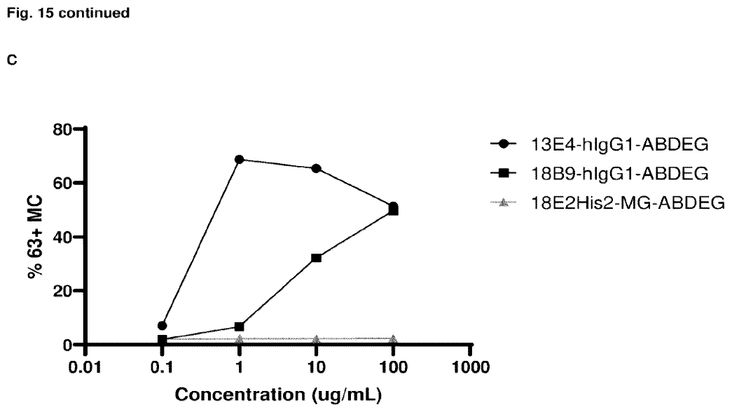

Bone marrow cells were isolated from hIgE/hFcεRIα mice. Cells were differentiated in RPMI+10% FBS+Glut+Pen/Strep+30 ng/mL IL-3 for 16 days. 3E+06/mL in 100 μL in 96-well bone marrow mast cells were sensitized with IgE at 3 μg/mL for 2.5 hours to load the receptor FcεRIα. After removal of IgE excess, varying concentrations of anti-IgE antibodies were added to sensitized cells for 30 minutes. 100 μL of cell suspension were transferred to 200 μL ice-cold FACS buffer to stop the degranulation reaction and CD63 recycling. Activated Mast cells were identified by looking at cKit+CD49b− (mast cells) CD63+ cells (activation marker). The results are shown in FIG. 15. Part A shows challenge with 20 μg/mL antibody and part B shows challenge with 200 μg/ml antibody. With the exception of antibody 13E4-hIgG1-ABDEG™ and 18B9-hIgG1-ABDEG, the various anti-IgE antibodies tested showed essentially no activation of mast cells, even at the higher concentration tested.

### B. Basophil Activation Test

Antibody binding to human IgE pre-bound on human basophils was analyzed by flow cytometry. Blood was obtained from a house dust-mite allergic donor. Basophil activation was measured according to FLOW CAST® Kit (BUHLMANN) in the presence of various anti-hIgE-ABDEG™ antibodies. The results were analyzed by flow cytometry and raw data were processed using FlowJo software. Basophil cells were identified as CCR3+ cells. Activated basophils were defined as CCR3+CD63+ cells. The percentage (%) of activated basophils and Stimulation Index (SI) is displayed in Table 38 below. An SI above 2 (% activated after challenge versus % activated at basal conditions) and basophil activation above 5% is considered positive.

TABLE 38
Basophil activation by anti-IgE antibodies

| | Challenge | % | |
|---|---|---|---|
| Antibody | concentration (μg/mL) | CD63+ | SI |
| 18B9-His- | 100 | 37.0 | 16.09 |
| WT | 20 | 12.6 | 5.48 |
| 13E4- | 100 | 68.6 | 27.44 |
| ABDEG ™ | 20 | 61.3 | 24.52 |
| 18E2VLHis- | 100 | 3.1 | 1.24 |
| ABDEG ™ | 20 | 3.7 | 1.48 |
| 18E2VHHis- | 100 | 4.8 | 1.92 |
| ABDEG ™ | 20 | 3.5 | 1.40 |
| 18E2His2- | 100 | 4.3 | 1.72 |
| ABDEG ™ | 20 | 4.0 | 1.60 |

Similar to the results seen with the mast activation experiment, antibodies based upon clones 18B9 and 13E4 demonstrated a degree of basophil activation. The various other anti-IgE ABDEG™ antibodies tested did not activate basophils.

### C. In Vivo Anaphylaxis

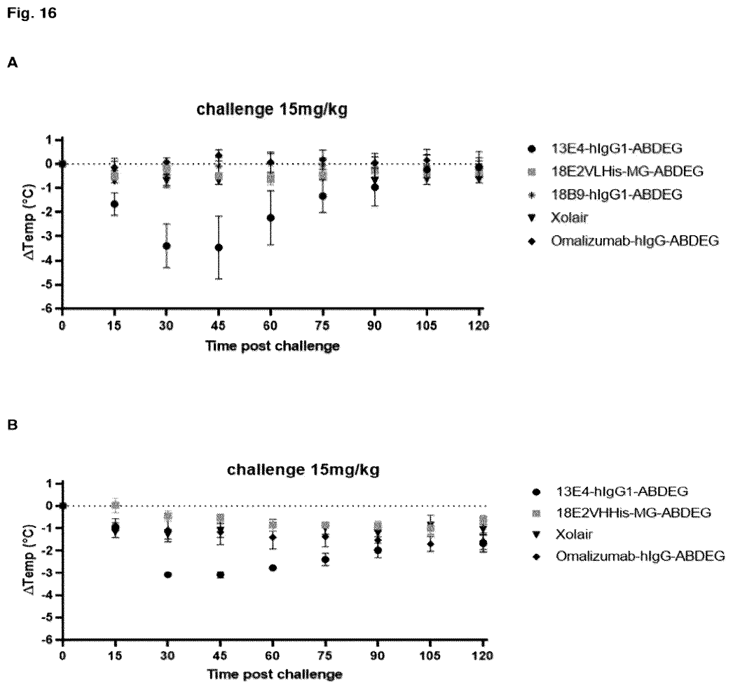

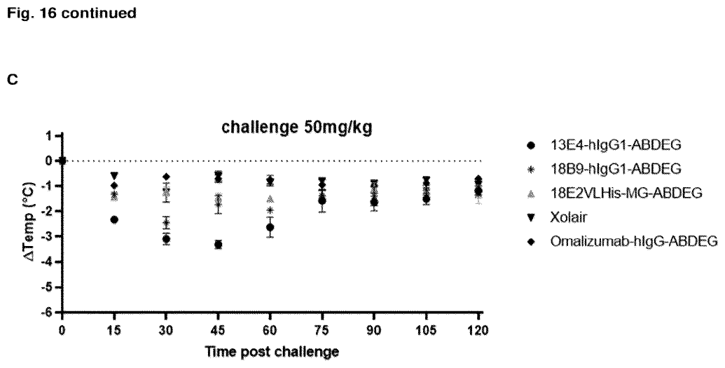

To assess the potential for an anaphylactic reaction in vivo, mice were challenged with various anti-IgE antibodies. On day −1, hFcεRIα/hIgE mice were sensitized by i.p. injection of recombinant human IgE at 15 mg/kg. One day later, mice were challenged i.v. with anti-IgE clones at 50 mg/kg or 15 mg/kg. Temperature was measured every 15 minutes for 2 hours. The results are shown in FIG. 16. Parts A and B show temperature changes over the time course of the experiment for antibodies administered at a dose of 15 mg/kg. Part C shows temperature changes over the time course of the experiment for antibodies administered at a dose of 50 mg/kg.

### Example 13 Inhibition of a Bullous Pemphigoid Disease Model Induced by IgG and IgE Autoantibodies

The ability of ABDEG™ antibodies to modify disease in vivo was assessed using a murine Bullous Pemphigoid BP disease model.

### A. IgG-Mediated BP Disease

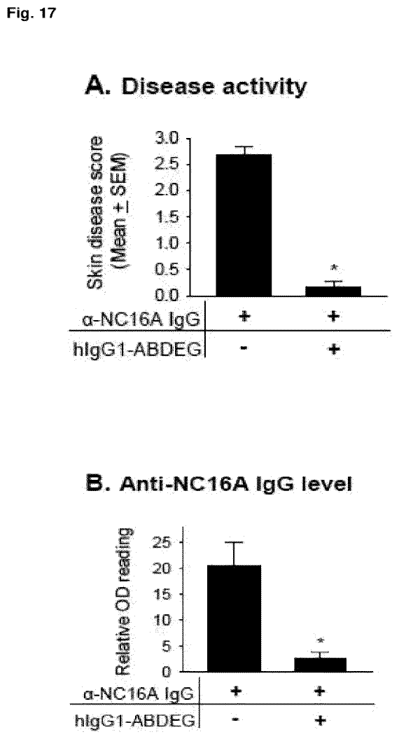

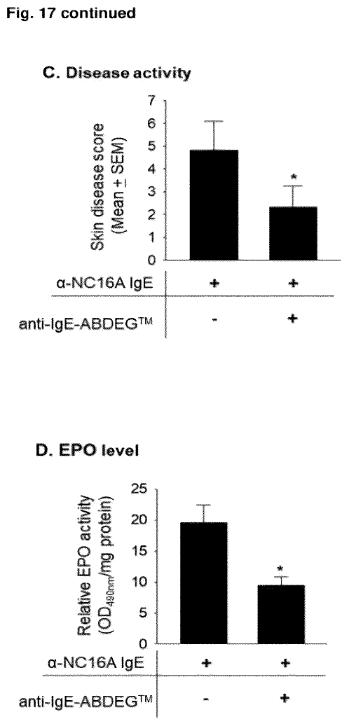

Eight week old human NC16A knock-in mice were injected i.p. with anti-hNC16A IgG (250 μg/g body weight) in the absence or presence of HEL-ABDEG™ (50 μg/g body weight) and examined 48h post injection. The results are shown in FIG. 17 parts (A) and (B). HEL-ABDEG™ significantly reduced skin disease severity (see FIG. 17A) and this was associated with a significantly reduced level of anti-NC16A IgG in the circulation (see FIG. 17B). *p<0.001, n=6 for each group.

### B. IgE-Mediated BP Disease

Eight week old hFcεRI/hNC16A mice were injected at ear pinna with anti-hNC16A IgE or control IgE (100 ng/g body weight), and then injected i.p. with 18E2VLHis-ABDEG™ (50 μg/g body weight). The mice were examined 48 h post IgE injection. The results are shown in FIG. 17 parts (C) and (D). Mice treated with 18E2VLHis-ABDEG™ exhibited significant reduction in clinical disease activity (see FIG. 17C), and this was associated with significantly reduced levels of eosinophil peroxidase (EPO) activity (indicative of a reduction in IgE) in the skin protein extracts (see FIG. 17D). *p<0.01, n=3-5 for each group.

1. A variant IgG Fc region consisting of two Fc domains, wherein one or both of the Fc domains comprise 252Y, 254T, 256E, 428L, 433K, and 434F, wherein the positions are defined in accordance with EU numbering.

2. The variant IgG Fc region of claim 1, wherein both of the Fc domains comprise 252Y, 254T, 256E, 428L, 433K, and 434F, wherein the positions are defined in accordance with EU numbering.

3. The variant IgG Fc region of claim 1, wherein both of the Fc domains are IgG1 Fc domains.

4. The variant IgG Fc region of claim 1, wherein both of the Fc domains are human IgG1 Fc domains.

5. The variant IgG Fc region of claim 1, wherein the variant IgG Fc region is a homodimer.

6. The variant IgG Fc region of claim 1, wherein the variant IgG Fc region binds to human FcRn with increased affinity at both pH 6.0 and pH 7.4.

7. The variant IgG Fc region of claim 1, wherein each of the Fc domains comprises no more than 10 amino acid substitutions as compared with the corresponding wild-type Fc domain.

8. The variant IgG Fc region of claim 1, wherein each of the Fc domains comprises no more than 6 amino acid substitutions as compared with the corresponding wild-type Fc domain.

9. A polynucleotide or plurality of polynucleotides which encode the variant IgG Fc region of claim 1.

10. An expression vector or plurality of expression vectors comprising the polynucleotide or plurality of polynucleotides of claim 9.

11. A host cell or cell-free expression system comprising the expression vector or plurality of expression vectors of claim 10.

12. A method of producing a variant IgG Fc region comprising culturing the host cell or cell-free expression system of claim 11 under conditions which permit expression of the variant IgG Fc region.

13. A composition comprising the variant IgG Fc region of claim 1.

14. A method of treating an antibody-mediated disorder in a subject comprising administering the variant IgG Fc region of claim 1 to the subject.

15. The method of claim 14, wherein the antibody-mediated disorder is an autoimmune disease.


SEQUENCE LISTING (216 sequences)

SEQ ID NO: 1 - protein, 221 aa, synthetic construct
  REGION 1..221: note = Synthetic peptide
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 2 - protein, 227 aa, synthetic construct
  REGION 1..227: note = Synthetic peptide
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 3 - protein, 226 aa, synthetic construct
  REGION 1..226: note = Synthetic peptide
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 4 - protein, 330 aa, synthetic construct
  REGION 1..330: note = Synthetic peptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 5 - protein, 221 aa, synthetic construct
  REGION 1..221: note = Synthetic peptide
CPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 6 - protein, 227 aa, synthetic construct
  REGION 1..227: note = Synthetic peptide
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 7 - protein, 226 aa, synthetic construct
  REGION 1..226: note = Synthetic peptide
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 8 - protein, 330 aa, synthetic construct
  REGION 1..330: note = Synthetic peptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 9 - protein, 5 aa, Lama glama
SYYMT

SEQ ID NO: 10 - protein, 17 aa, Lama glama
SIYSDGSNTYYADSVKG

SEQ ID NO: 11 - protein, 17 aa, Lama glama
DLKARYSGSYHDEGYDY

SEQ ID NO: 12 - protein, 5 aa, Lama glama
SYYMS

SEQ ID NO: 13 - protein, 17 aa, Lama glama
SIYSDGSYAYYADSVKG

SEQ ID NO: 14 - protein, 17 aa, Lama glama
DLKARYSGTYHDEGYDY

SEQ ID NO: 15 - protein, 5 aa, Lama glama
NYAMS

SEQ ID NO: 16 - protein, 17 aa, Lama glama
AISWNGGSTYYAESMKG

SEQ ID NO: 17 - protein, 12 aa, Lama glama
DLLVAARGGMDY

SEQ ID NO: 18 - protein, 19 aa, Lama glama
SIYSDGRGSKTFYADSVKG

SEQ ID NO: 19 - protein, 10 aa, Lama glama
DLLVAARGSM

SEQ ID NO: 20 - protein, 5 aa, Lama glama
SYVMS

SEQ ID NO: 21 - protein, 17 aa, Lama glama
SIYHDGSHTYYADFVKG

SEQ ID NO: 22 - protein, 17 aa, Lama glama
GTSYSGSYYYTDPFFGS

SEQ ID NO: 23 - protein, 17 aa, Lama glama
SIYSDGSHTYYADSVKG

SEQ ID NO: 24 - protein, 18 aa, Lama glama
NLEHYSGSYYYTDPRYDY

SEQ ID NO: 25 - protein, 5 aa, Lama glama
SYVMT

SEQ ID NO: 26 - protein, 17 aa, Lama glama
SIYSDGSHTYYADSVKD

SEQ ID NO: 27 - protein, 18 aa, Lama glama
DAEYYSGSYYYTDTKYDY

SEQ ID NO: 28 - protein, 5 aa, Lama glama
DYDMS

SEQ ID NO: 29 - protein, 17 aa, Lama glama
IISWNGGSTDYAESMKG

SEQ ID NO: 30 - protein, 10 aa, Lama glama
HSVGRNGYDY

SEQ ID NO: 31 - protein, 5 aa, Lama glama
NYYMS

SEQ ID NO: 32 - protein, 17 aa, Lama glama
SIYSDGGYTYYADSVKG

SEQ ID NO: 33 - protein, 17 aa, Lama glama
DLKPRNSGTYHDEGYDD

SEQ ID NO: 34 - protein, 5 aa, Lama glama
TYVMS

SEQ ID NO: 35 - protein, 7 aa, Lama glama
TSYYAWN

SEQ ID NO: 36 - protein, 16 aa, Lama glama
VIAYDGSTDYSPSLKS

SEQ ID NO: 37 - protein, 14 aa, Lama glama
DYRINSDYAGGYDY

SEQ ID NO: 38 - protein, 17 aa, Lama glama
GTSYSASYYYTDPFFGS

SEQ ID NO: 39 - protein, 17 aa, Lama glama
SISSDGSNPYYADSVKG

SEQ ID NO: 40 - protein, 15 aa, Lama glama
DTLTGASYSDSLYDY

SEQ ID NO: 41 - protein, 5 aa, Lama glama
SYAMS

SEQ ID NO: 42 - protein, 17 aa, Lama glama
SIYSYSSNTYYADSVKG

SEQ ID NO: 43 - protein, 15 aa, Lama glama
TTLSRLTYSDYRYDY

SEQ ID NO: 44 - protein, 17 aa, Lama glama
SIYSDDSNTDYADSVKG

SEQ ID NO: 45 - protein, 15 aa, Lama glama
ATGTVGYYSDYFYDY

SEQ ID NO: 46 - protein, 5 aa, Lama glama
DYAMS

SEQ ID NO: 47 - protein, 17 aa, Lama glama
GISWKGGIIYYAESMEG

SEQ ID NO: 48 - protein, 12 aa, Lama glama
ALGTVASGQYDY

SEQ ID NO: 49 - protein, 17 aa, Lama glama
SISSDGSNTYYADSVKG

SEQ ID NO: 50 - protein, 14 aa, Lama glama
DDNSGSDYEFGYDY

SEQ ID NO: 51 - protein, 7 aa, Lama glama
SSYYDWT

SEQ ID NO: 52 - protein, 16 aa, Lama glama
VIHYDGSTYYSPSLKS

SEQ ID NO: 53 - protein, 11 aa, Lama glama
SYSSSPWDYDY

SEQ ID NO: 54 - protein, 11 aa, Lama glama
QGGSLGSSYAH

SEQ ID NO: 55 - protein, 7 aa, Lama glama
DDDSRPS

SEQ ID NO: 56 - protein, 10 aa, Lama glama
QSADSSGNPV

SEQ ID NO: 57 - protein, 11 aa, Lama glama
QGGSLGATYAY

SEQ ID NO: 58 - protein, 10 aa, Lama glama
QSAYSNGNAV

SEQ ID NO: 59 - protein, 11 aa, Lama glama
QGGTLGSYGAH

SEQ ID NO: 60 - protein, 7 aa, Lama glama
GDNSRPS

SEQ ID NO: 61 - protein, 10 aa, Lama glama
QSFDYSGNAV

SEQ ID NO: 62 - protein, 11 aa, Lama glama
QGGSLGSNYAY

SEQ ID NO: 63 - protein, 10 aa, Lama glama
QSADSNGNAV

SEQ ID NO: 64 - protein, 11 aa, Lama glama
QGGSLGSSYVH

SEQ ID NO: 65 - protein, 7 aa, Lama glama
DGDSRPS

SEQ ID NO: 66 - protein, 10 aa, Lama glama
QSADSSGNAV

SEQ ID NO: 67 - protein, 7 aa, Lama glama
ADDSRPS

SEQ ID NO: 68 - protein, 11 aa, Lama glama
QGDRLGSRYIY

SEQ ID NO: 69 - protein, 7 aa, Lama glama
DDDRRPS

SEQ ID NO: 70 - protein, 11 aa, Lama glama
QGGSLGTSYAY

SEQ ID NO: 71 - protein, 7 aa, Lama glama
DDDNRPS

SEQ ID NO: 72 - protein, 10 aa, Lama glama
QSEDTSSNFV

SEQ ID NO: 73 - protein, 14 aa, Lama glama
TGSSSNIGGGYYLS

SEQ ID NO: 74 - protein, 7 aa, Lama glama
NANNRAS

SEQ ID NO: 75 - protein, 11 aa, Lama glama
GCYDSSLSTPV

SEQ ID NO: 76 - protein, 11 aa, Lama glama
QGGSLGGSYAH

SEQ ID NO: 77 - protein, 7 aa, Lama glama
DDTSRPS

SEQ ID NO: 78 - protein, 10 aa, Lama glama
QSSYSSGNPV

SEQ ID NO: 79 - protein, 11 aa, Lama glama
QGDNLGNNYVQ

SEQ ID NO: 80 - protein, 7 aa, Lama glama
DDNRRPS

SEQ ID NO: 81 - protein, 10 aa, Lama glama
QASDSSGNAV

SEQ ID NO: 82 - protein, 11 aa, Lama glama
QGGNLGSSYAH

SEQ ID NO: 83 - protein, 14 aa, Lama glama
AGTSNDVGYGNYVS

SEQ ID NO: 84 - protein, 7 aa, Lama glama
DVNKRAS

SEQ ID NO: 85 - protein, 10 aa, Lama glama
ASYRTNNNVV

SEQ ID NO: 86 - protein, 11 aa, Lama glama
QGDNFGSYYAS

SEQ ID NO: 87 - protein, 7 aa, Lama glama
KDSERPS

SEQ ID NO: 88 - protein, 10 aa, Lama glama
LSYDNNGAPV

SEQ ID NO: 89 - protein, 14 aa, Lama glama
AGTSSDIGGYNSVS

SEQ ID NO: 90 - protein, 7 aa, Lama glama
EVNKRAS

SEQ ID NO: 91 - protein, 10 aa, Lama glama
ASYRNSNNVV

SEQ ID NO: 92 - protein, 126 aa, Lama glama
QVQLVESGGGLVQPGGSLRLSCAASGFTFSSYYMTWVRQAPGKGLEWVSSIYSDGSNTYYADSVKGRFTISRDNAKNTLHLQMNSLKSEDTAVYYCAKDLKARYSGSYHDEGYDYWGQGTQVTVSS

SEQ ID NO: 93 - protein, 107 aa, Lama glama
QSALTQPSALSVTLGQTAKITCQGGSLGSSYAHWYQQKPGQAPVLVIYDDDSRPSGIPERFSGSSSGGRATLTISGAQAEDEGDYYCQSADSSGNPVFGGGTKLTVL

SEQ ID NO: 94 - protein, 126 aa, Lama glama
EVQLVESGGGLVQPGGSLRLSCAASGFTFSSYYMSWVRQAPGKGLEWVSSIYSDGSYAYYADSVKGRFTISRDNAKNTLYLQMNSLKSEDTAVYYCAKDLKARYSGTYHDEGYDYWGQGTQVTVSS

SEQ ID NO: 95 - protein, 107 aa, Lama glama
SSALTQPSALSVTLGQSAKITCQGGSLGATYAYWYQQKPGQAPVLVIYDDDSRPSGIPERFSGSSSGGRATLTISGAQAEDEGDYYCQSAYSNGNAVFGGGTHLTVL

SEQ ID NO: 96 - protein, 121 aa, Lama glama
EVQVQESGGGLVQPGGSLRLSCAASGFTFDNYAMSWVRQAPGKGLEWVSAISWNGGSTYYAESMKGRFTISRDNAKNMLYLQMNSLKSEDTAVYYCAKDLLVAARGGMDYWGKGTLVTVSS

SEQ ID NO: 97 - protein, 107 aa, Lama glama
SSALTQPSAVSVSLEQTARITCQGGTLGSYGAHWYQQKPGQAPVLLIYGDNSRPSGIPERFSGTRSGGTATLTISGAQAEDEADYYCQSFDYSGNAVFGGGTHLTVL

SEQ ID NO: 98 - protein, 123 aa, Lama glama
QLQVVESGGGLVQPGGSLRLSCAASGFTFSSYYMSWVRQAPGKGLEWVSSIYSDGRGSKTFYADSVKGRFTISRDNAKNTLYLQMNSLKSEDTAVYFCAKDLLVAARGSMDYWGQGTQVTVSS

SEQ ID NO: 99 - protein, 107 aa, Lama glama
NFMLTQPSAVSVSLEQTARITCQGGTLGSYGAHWYQQKPGQAPVLLIYGDNSRPSGIPERFSGTRSGGTATLTISGAQAEDEADYYCQSFDYSGNAVFGGGTHLTVL

SEQ ID NO: 100 - protein, 126 aa, Lama glama
ELQLVESGGGLVQPGGSLRLSCAASGFTFSSYVMSWVRQAPGKGLEWVSSIYHDGSHTYYADFVKGRFTISRDNAKNTLYLQMNSLKSEDTAVYYCASGTSYSGSYYYTDPFFGSWGQGTQVTVSS

SEQ ID NO: 101 - protein, 107 aa, Lama glama
HSAVTQPSALSVTLGQTAKITCQGGSLGSNYAYWYQQKPGQAPVLVIYDDDSRPSGIPERFSGSSSGGTATLTISGAQAEDEGDYYCQSADSNGNAVFGGGTHLTVL

SEQ ID NO: 102 - protein, 127 aa, Lama glama
QVQLVESGGGLVQPGGSLRLSCAASGFTFSSYVMSWVRQAPGKGLEWVSSIYSDGSHTYYADSVKGRFTISRDNAKNTLYLQMNSLKSEDTAVYYCAKNLEHYSGSYYYTDPRYDYWGQGTQVTVSS

SEQ ID NO: 103 - protein, 107 aa, Lama glama
SSALTQPSALSVTLGQTAKITCQGGSLGSSYVHWYQQKPGQAPVLVIYDGDSRPSGIPERFSGSSSGGTATLTISGAQAEDEDDYYCQSADSSGNAVFGGGTHLTVL

SEQ ID NO: 104 - protein, 127 aa, Lama glama
QLQLVESGGGLVQPGGSLRLSCAASGFAFSSYVMTWVRQAPGKGLEWVSSIYSDGSHTYYADSVKDRFTISRDNAKNTLFLQMNSLKSEDTAVYYCAKDAEYYSGSYYYTDTKYDYWGQGTQVTVSS

SEQ ID NO: 105 - protein, 107 aa, Lama glama
SSALTQPSALSVTLGQTAKITCQGGSLGSSYAHWYQQKPGQAPVLVIYADDSRPSGIPERFSGSSSGGTATLTISGAQAEDEGDYYCQSADSSGNAVFGGGTHLTVL

SEQ ID NO: 106 - protein, 126 aa, Lama glama
QLQLVESGGGLVQPGGSLRLSCAASGFTFSSYVMSWVRQAPGKGLEWVSSIYHDGSHTYYADFVKGRFTISRDNAKNTLYLQMNSLKSEDTAVYYCASGTSYSGSYYYTDPFFGSWGQGTQVTVSS

SEQ ID NO: 107 - protein, 107 aa, Lama glama
NFMLTQPSALSVTLGQTARITCQGDRLGSRYIYWYQQKPPQAPVLVIHDDDRRPSGIPERFSGSSSGGTATLTISGAQAEDDGDYYCQSADSSGNPVFGGGTHLTVL

SEQ ID NO: 108 - protein, 119 aa, Lama glama
EVQVQESGGGLVQPGGSLRLSCAASGFTFDDYDMSWVRQAPGKGLEWVSIISWNGGSTDYAESMKGRFTISRDNAKNTLYLQMNSLKSEDTAVYFCAKHSVGRNGYDYWGQGTQVTVSS

SEQ ID NO: 109 - protein, 107 aa, Lama glama
NFMLTQPSALSVTLGQTAKITCQGGSLGTSYAYWYQQKAGQAPVVVIYDDDNRPSGIPERFSGSSSGGTATLTISGAQAEDEGDYYCQSEDTSSNFVFGGGTHLTVL

SEQ ID NO: 110 - protein, 126 aa, Lama glama
ELQLVESGGGLVRPGGSLRLSCAASGFTFSNYYMSWVRQAPGKGLEWLSSIYSDGGYTYYADSVKGRFTISRDNAKNTLYLQMNSLKSEDTAVYYCAKDLKPRNSGTYHDEGYDDWGQGTQVTVSS

SEQ ID NO: 111 - protein, 107 aa, Lama glama
SSELTQASALSVTLGQTAKITCQGGSLGSSYAHWYQQKPGQAPVLVIYDDDSRPSGIPERFSGSSSGGRATLTISGAQAEDEGDYYCQSADSSGNPVFGGGTKLTVL

SEQ ID NO: 112 - protein, 126 aa, Lama glama
EVQLVESGGGLVQPGGSLRLSCAASGFTFSTYVMSWVRQAPGKGLEWVSSIYSDGSHTYYADSVKGRFTISRDNAKNTLYLQMNSLKSEDTAMYYCTTGTSYSGSYYYTDPFFGSWGQGTQVIVSS

SEQ ID NO: 113 - protein, 107 aa, Lama glama
HSAVTQPSALSVTLGQTAKITCQGGSLGSNYAYWYQQKPGQAPVLVIYDDDSRPSGIPERFSGSSSGGTATLTISGAQAEDEGDYYCQSADSNGNAVFGGGTHLTVM

SEQ ID NO: 114 - protein, 124 aa, Lama glama
QVQVQESGPGLVKPSQTLSLTCTVSGGSITTSYYAWNWIRQPPGKGLEWMGVIAYDGSTDYSPSLKSRTSISRDTSKNQFSLQLSSVTPEDTAVYYCARDYRINSDYAGGYDYWGQGTQVTVSS

SEQ ID NO: 115 - protein, 111 aa, Lama glama
QPVLNQLSSMSGSPGQTVTITCTGSSSNIGGGYYLSWYQQLPGTAPKLLIYNANNRASGVPNRFSGSKTGSLASLTITGLQAEDEADYYCGCYDSSLSTPVFGGGTKLIVL

SEQ ID NO: 116 - protein, 126 aa, Lama glama
EVQLVESGGGLVQPGGSLRLSCATSGFTFSSYVMSWVRQAPGKGLEWVSSIYHDGSHTYYADFVKGRFTISRDNAKNTLYLQMNSLKSEDTAVYYCASGTSYSASYYYTDPFFGSWGQGTQVTVSS

SEQ ID NO: 117 - protein, 107 aa, Lama glama
SYELTQPSALSVTLGQTAKITCQGGSLGSNYAYWYQQKPGQAPVLVIYDDDSRPSGIPERFSGSSSGGTATLTISGAQAEDEGDYYCQSADSNGNAVFGGGTHLTVL

SEQ ID NO: 118 - protein, 124 aa, Lama glama
QVQVEESGGGLVQPGGSLRLSCAASGFTFSSYYMSWVRQAPGKGLEWVSSISSDGSNPYYADSVKGRFTISRDNAKNTLYLQMNSLKSEDTAVYYCAKDTLTGASYSDSLYDYWGQGTQVTVSS

SEQ ID NO: 119 - protein, 107 aa, Lama glama
SSALTQPSALSVTLGQTADITCQGGSLGGSYAHWYQQKPGQAPMLVIYDDTSRPSGIPERFSGSSSGDRVTLTISGAQAEDGGDYYCQSSYSSGNPVFGGGTKLTVL

SEQ ID NO: 120 - protein, 124 aa, Lama glama
QVQLVESGGGLVQPGGSLRLSCAASGFTFSSYAMSWVRQAPGKGLEMVSSIYSYSSNTYYADSVKGRFTISRDNAKNTLYLQMNSLKSEDTAVYYCAKTTLSRLTYSDYRYDYWGQGTQVTVSS

SEQ ID NO: 121 - protein, 107 aa, Lama glama
SYELTQPSALSVTLRQTAKITCQGDNLGNNYVQWYQQKPGQAPELVIYDDNRRPSGIPERFSGSSSGGTATLTISGAQADDEGDYYCQASDSSGNAVVGGGTHLIIL

SEQ ID NO: 122 - protein, 126 aa, Lama glama
QLQVVESGGGLVQPGGSLRLSCAASGFTFSSYYMSWVRQAPGKGLEWVSSIYSDGSYAYYADSVKGRFTISRDNAKNTLYLQMNSLKSEDTAVYYCAKDLKARYSGTYHDEGYDYWGQGTQVTVSS

SEQ ID NO: 123 - protein, 107 aa, Lama glama
QSALTQPSALSVTLGQTAKITCQGGNLGSSYAHWYQQKPGQAPVLVIYDDDSRPSGIPERFSGSSSGGTATLIISGAQAEDEGDYYCQSADSSGNPVFGGGTKLTVL

SEQ ID NO: 124 - protein, 124 aa, Lama glama
EVQLVESGGGLVQPGGSLRLSCAASGFTFSSYYMSWVRQAPGKGLERVSSIYSDDSNTDYADSVKGRFTISRDNAKNTLYLQMNSLKSEDTAVYYCAKATGTVGYYSDYFYDYWGQGTQVTVSS

SEQ ID NO: 125 - protein, 107 aa, Lama glama
NFMLTQPSALSVTLGQTAKITCQGGSLGSSYAHWYQQKPGQAPVLVIYDDDSRPSGIPERFSGSSSGGTATLTISGAQAEDEGDYYCQSADSSGNAVFGGGTHLTVL

SEQ ID NO: 126 - protein, 121 aa, Lama glama
EVQLVESGGGLVQPGGSLRLSCAASGFTFDDYAMSWVRQAPGKGLEWVSGISWKGGIIYYAESMEGRFTISRDNAKNTLYLQMNSLKSEDTAVYYCAKALGTVASGQYDYWGQGTQVTVSS

SEQ ID NO: 127 - protein, 110 aa, Lama glama
SSALTQPPSVSGSPGKTVTISCAGTSNDVGYGNYVSWYQQLPGMAPKLLIYDVNKRASGITDRFSGSKSGNTASLTISGLQSEDEADYYCASYRTNNNVVFGGGTKVTVL

SEQ ID NO: 128 - protein, 123 aa, Lama glama
QLQLVESGGGLVQPGGSLRLSCAASGFTFSSYYMSWVRQAPGKGLEWVFSISSDGSNTYYADSVKGRFTISRDNAKNTLYLQMNSLKSEDTAVYYCAKDDNSGSDYEFGYDYWGQGTQVTVSS

SEQ ID NO: 129 - protein, 107 aa, Lama glama
SYELTQPSAVSVSLGQTARITCQGDNFGSYYASWYQQKSGQAPVRVIYKDSERPSGIPERFSGSSSGDTATLTISGAQFEDEADYYCLSYDNNGAPVFGGGTKLTVL

SEQ ID NO: 130 - protein, 121 aa, Lama glama
ELQLVESGPGLVKPSQTLSLTCTVSGASITSSYYDWTWIRQPPGKGLEWMGVIHYDGSTYYSPSLKSRTSISRDTSKNQFSLQLSSVTPEDTAVYYCTQSYSSSPWDYDYWGQGTQVTVSS

SEQ ID NO: 131 - protein, 110 aa, Lama glama
QAVLTQPPSVSGTLGKTLTISCAGTSSDIGGYNSVSWYQQLPGTAPKLLIYEVNKRASGIPDRFSGSKSGNTASLSISGLQSEDEADYYCASYRNSNNVVFGGGTHLTVL

SEQ ID NO: 132 - protein, 5 aa, synthetic construct
  REGION 1..5: note = Synthetic peptide
SYVMH

SEQ ID NO: 133 - protein, 5 aa, synthetic construct
  REGION 1..5: note = Synthetic peptide
SYVMH

SEQ ID NO: 134 - protein, 5 aa, synthetic construct
  REGION 1..5: note = Synthetic peptide
SYYMH

SEQ ID NO: 135 - protein, 11 aa, synthetic construct
  REGION 1..11: note = Synthetic peptide
QGDRLGSRYIH

SEQ ID NO: 136 - protein, 10 aa, synthetic construct
  REGION 1..10: note = Synthetic peptide
HSFDYSGNAV

SEQ ID NO: 137 - protein, 126 aa, synthetic construct
  REGION 1..126: note = Synthetic peptide
QLQLVESGGGLVQPGGSLRLSCAASGFTFSSYVMHWVRQAPGKGLEWVSSIYHDGSHTYYADFVKGRFTISRDNAKNTLYLQMNSLKSEDTAVYYCASGTSYSGSYYYTDPFFGSWGQGTQVTVSS

SEQ ID NO: 138 - protein, 107 aa, synthetic construct
  REGION 1..107: note = Synthetic peptide
NFMLTQPSALSVTLGQTARITCQGDRLGSRYIHWYQQKPPQAPVLVIHDDDRRPSGIPERFSGSSSGGTATLTISGAQAEDDGDYYCQSADSSGNPVFGGGTHLTVL

SEQ ID NO: 139 - protein, 127 aa, synthetic construct
  REGION 1..127: note = Synthetic peptide
QVQLVESGGGLVQPGGSLRLSCAASGFTFSSYVMHWVRQAPGKGLEWVSSIYSDGSHTYYADSVKGRFTISRDNAKNTLYLQMNSLKSEDTAVYYCAKNLEHYSGSYYYTDPRYDYWGQGTQVTVSS

SEQ ID NO: 140 - protein, 107 aa, synthetic construct
  REGION 1..107: note = Synthetic peptide
SSALTQPSALSVTLGQTAKITCQGGSLGSSYVHWYQQKPGQAPVLVIHDGDSRPSGIPERFSGSSSGGTATLTISGAQAEDEDDYYCQSADSSGNAVFGGGTHLTVL

SEQ ID NO: 141 - protein, 123 aa, synthetic construct
  REGION 1..123: note = Synthetic peptide
QLQVVESGGGLVQPGGSLRLSCAASGFTFSSYYMHWVRQAPGKGLEWVSSIYSDGRGSKTFYADSVKGRFTISRDNAKNTLYLQMNSLKSEDTAVYFCAKDLLVAARGSMDYWGQGTQVTVSS

SEQ ID NO: 142 - protein, 107 aa, synthetic construct
  REGION 1..107: note = Synthetic peptide
NFMLTQPSAVSVSLEQTARITCQGGTLGSYGAHWYQQKPGQAPVLLIHGDNSRPSGIPERFSGTRSGGTATLTISGAQAEDEADYYCHSFDYSGNAVFGGGTHLTVL

SEQ ID NO: 143 - protein, 6 aa, synthetic construct
  REGION 1..6: note = Synthetic peptide
SGYSWN

SEQ ID NO: 144 - protein, 16 aa, synthetic construct
  REGION 1..16: note = Synthetic peptide
SITYDGSTNYNPSVKG

SEQ ID NO: 145 - protein, 12 aa, synthetic construct
  REGION 1..12: note = Synthetic peptide
GSHYFGHWHFAV

SEQ ID NO: 146 - protein, 121 aa, synthetic construct [sequence omitted]

SEQ ID NO: 147 - protein, 15 aa, synthetic construct
  REGION 1..15: note = Synthetic peptide
RASQSVDYDGDSYMN

SEQ ID NO: 148 - protein, 7 aa, synthetic construct
  REGION 1..7: note = Synthetic peptide
AASYLES

SEQ ID NO: 149 - protein, 9 aa, synthetic construct
  REGION 1..9: note = Synthetic peptide
QQSHEDPYT

SEQ ID NO: 150 - protein, 111 aa, synthetic construct [sequence omitted]

SEQ ID NO: 151 - protein, 5 aa, synthetic construct
  REGION 1..5: note = Synthetic peptide
WYWLE

SEQ ID NO: 152 - protein, 17 aa, synthetic construct
  REGION 1..17: note = Synthetic peptide
EIDPGTFTTNYNEKFKA

SEQ ID NO: 153 - protein, 14 aa, synthetic construct
  REGION 1..14: note = Synthetic peptide
FSHFSGSNYDYFDY

SEQ ID NO: 154 - protein, 123 aa, synthetic construct
  REGION 1..123: note = Synthetic peptide
QVQLVQSGAEVMKPGSSVKVSCKASGYTFSWYWLEWVRQAPGHGLEWMGEIDPGTFTTNYNEKFKARVTFTADTSTSTAYMELSSLRSEDTAVYYCARFSHFSGSNYDYFDYWGQGTLVTVSS

SEQ ID NO: 155 - protein, 11 aa, synthetic construct
  REGION 1..11: note = Synthetic peptide
RASQSIGTNIH

SEQ ID NO: 156 - protein, 7 aa, synthetic construct
  REGION 1..7: note = Synthetic peptide
YASESIS

SEQ ID NO: 157 - protein, 9 aa, synthetic construct
  REGION 1..9: note = Synthetic peptide
QQSWSWPTT

SEQ ID NO: 158 - protein, 107 aa, synthetic construct
  REGION 1..107: note = Synthetic peptide
EIVMTQSPATLSVSPGERATLSCRASQSIGTNIHWYQQKPGQAPRLLIYYASESISGIPARFSGSGSGTEFTLTISSLQSEDFAVYYCQQSWSWPTTFGGGTKVEIK

SEQ ID NO: 159 - protein, 10 aa, Homo sapiens
EPKSCDKTHT

SEQ ID NO: 160 - protein, 5 aa, Homo sapiens
CPPCP

SEQ ID NO: 161 - protein, 8 aa, Homo sapiens
APELLGGP

SEQ ID NO: 162 - protein, 12 aa, Homo sapiens
ELKTPLGDTTHT

SEQ ID NO: 163 - protein, 50 aa, Homo sapiens
CPRCPEPKSCDTPPPCPRCPEPKSCDTPPPCPRCPEPKSCDTPPPCPRCP

SEQ ID NO: 164 - protein, 8 aa, Homo sapiens
APELLGGP

SEQ ID NO: 165 - protein, 7 aa, Homo sapiens
ESKYGPP

SEQ ID NO: 166 - protein, 5 aa, Homo sapiens
CPSCP

SEQ ID NO: 167 - protein, 8 aa, Homo sapiens
APEFLGGP

SEQ ID NO: 168 - skipped

SEQ ID NO: 169 - protein, 10 aa, Homo sapiens
CCVECPPPCP

SEQ ID NO: 170 - protein, 7 aa, Homo sapiens
APPVAGP

SEQ ID NO: 171 - protein, 126 aa, synthetic construct
  REGION 1..126: note = Synthetic peptide
EVQLLESGGGLVQPGGSLRLSCAASGFTFSSYVMSWVRQAPGKGLEWVSSIYHDGSHTYYADFVKGRFTISRDNSKNTLYLQMNSLRAEDTAVYYCAKGTSYSGSYYYTDPFFGSWGQGTLVTVSS

SEQ ID NO: 172 - protein, 107 aa, synthetic construct
  REGION 1..107: note = Synthetic peptide
SSELTQDPAVSVALGQTVRITCQGGSLGSNYAYWYQQKPGQAPVLVIYDDDSRPSGIPDRFSGSSSGNTASLTITGAQAEDEADYYCQSADSNGNAVFGGGTQLTVL

SEQ ID NO: 173 - protein, 126 aa, synthetic construct
  REGION 1..126: note = Synthetic peptide
EVQLLESGGGLVQPGGSLRLSCAASGFTFSSYVMHWVRQAPGKGLEWVSSIYHDGSHTYYADFVKGRFTISRDNSKNTLYLQMNSLRAEDTAVYYCAKGTSYSGSYYYTDPFFGSWGQGTLVTVSS

SEQ ID NO: 174 - protein, 107 aa, synthetic construct
  REGION 1..107: note = Synthetic peptide
SSELTQDPAVSVALGQTVRITCQGDRLGSRYIHWYQQKPGQAPVLVIYDDDRRPSGIPDRFSGSSSGNTASLTITGAQAEDEADYYCQSADSSGNPVFGGGTQLTVL

SEQ ID NO: 175 - protein, 127 aa, synthetic construct
  REGION 1..127: note = Synthetic peptide
EVQLLESGGGLVQPGGSLRLSCAASGFTFSSYVMSWVRQAPGKGLEWVSSIYSDGSHTYYADSVKGRFTISRDNSKNTLYLQMNSLRAEDTAVYYCAKNLEHYSGSYYYTDPRYDYWGQGTLVTVSS

SEQ ID NO: 176 - protein, 107 aa, synthetic construct
  REGION 1..107: note = Synthetic peptide
SSELTQDPAVSVALGQTVRITCQGGSLGSSYVHWYQQKPGQAPVLVIYDGDSRPSGIPDRFSGSSSGNTASLTITGAQAEDEADYYCQSADSSGNAVFGGGTQLTVL

SEQ ID NO: 177 - protein, 17 aa, synthetic construct
  REGION 1..17: note = Synthetic peptide
EIDPGTFTTHYNEKFKA

SEQ ID NO: 178 - protein, 14 aa, synthetic construct
  REGION 1..14: note = Synthetic peptide
FSHFSGSHYDYFDY

SEQ ID NO: 179 - protein, 5 aa, synthetic construct
  REGION 1..5: note = Synthetic peptide
WYHLE

SEQ ID NO: 180 - protein, 14 aa, synthetic construct
  REGION 1..14: note = Synthetic peptide
FSHFSGSNHDYFDY

SEQ ID NO: 181 - protein, 14 aa, synthetic construct
  REGION 1..14: note = Synthetic peptide
FSHFSGHNYDYFDY

SEQ ID NO: 182 - protein, 11 aa, synthetic construct
  REGION 1..11: note = Synthetic peptide
RASQHIGTNIH

SEQ ID NO: 183 - protein, 9 aa, synthetic construct
  REGION 1..9: note = Synthetic peptide
QQSWSHPTT

SEQ ID NO: 184 - protein, 7 aa, synthetic construct
  REGION 1..7: note = Synthetic peptide
HASESIS

SEQ ID NO: 185 - protein, 123 aa, synthetic construct
  REGION 1..123: note = Synthetic peptide
QVQLVQSGAEVMKPGSSVKVSCKASGYTFSWYWLEWVRQAPGHGLEWMGEIDPGTFTTHYNEKFKARVTFTADTSTSTAYMELSSLRSEDTAVYYCARFSHFSGSHYDYFDYWGQGTLVTVSS

SEQ ID NO: 186 - protein, 123 aa, synthetic construct
  REGION 1..123: note = Synthetic peptide
QVQLVQSGAEVMKPGSSVKVSCKASGYTFSWYHLEWVRQAPGHGLEWMGEIDPGTFTTNYNEKFKARVTFTADTSTSTAYMELSSLRSEDTAVYYCARFSHFSGSNHDYFDYWGQGTLVTVSS

SEQ ID NO: 187 - protein, 123 aa, synthetic construct
  REGION 1..123: note = Synthetic peptide
QVQLVQSGAEVMKPGSSVKVSCKASGYTFSWYHLEWVRQAPGHGLEWMGEIDPGTFTTNYNEKFKARVTFTADTSTSTAYMELSSLRSEDTAVYYCARFSHFSGSHYDYFDYWGQGTLVTVSS

SEQ ID NO: 188 - protein, 123 aa, synthetic construct
  REGION 1..123: note = Synthetic peptide
QVQLVQSGAEVMKPGSSVKVSCKASGYTFSWYHLEWVRQAPGHGLEWMGEIDPGTFTTNYNEKFKARVTFTADTSTSTAYMELSSLRSEDTAVYYCARFSHFSGHNYDYFDYWGQGTLVTVSS

SEQ ID NO: 189 - protein, 107 aa, synthetic construct
  REGION 1..107: note = Synthetic peptide
EIVMTQSPATLSVSPGERATLSCRASQHIGTNIHWYQQKPGQAPRLLIYYASESISGIPARFSGSGSGTEFTLTISSLQSEDFAVYYCQQSWSHPTTFGGGTKVEIK

SEQ ID NO: 190 - protein, 107 aa, synthetic construct
  REGION 1..107: note = Synthetic peptide
EIVMTQSPATLSVSPGERATLSCRASQSIGTNIHWYQQKPGQAPRLLIYHASESISGIPARFSGSGSGTEFTLTISSLQSEDFAVYYCHQSDSWPTTFGGGTKVEIK

SEQ ID NO: 191 - protein, 107 aa, synthetic construct
  REGION 1..107: note = Synthetic peptide
EIVMTQSPATLSVSPGERATLSCRASQSIGTNIHWYQQKPGQAPRLLIYHASESISGIPARFSGSGSGTEFTLTISSLQSEDFAVYYCQQSWSHPTTFGGGTKVEIK

SEQ ID NO: 192 - protein, 107 aa, synthetic construct
  REGION 1..107: note = Synthetic peptide
EIVMTQSPATLSVSPGERATLSCRASQSIGTNIHWYQQKPGQAPRLLIYHASESISGIPARFSGSGSGTEFTLTISSLQSEDFAVYYCQQSDSHPTTFGGGTKVEIK

SEQ ID NO: 193 - protein, 107 aa, synthetic construct
  REGION 1..107: note = Synthetic peptide
EIVMTQSPATLSVSPGERATLSCRASQSIGTNIHWYQQKPGQAPRLLIYHASESISGIPARFSGSGSGTEFTLTISSLQSEDFAVYYCQQSWSWPTTFGGGTKVEIK

SEQ ID NO: 194 - protein, 107 aa, synthetic construct
  REGION 1..107: note = Synthetic peptide
EIVMTQSPATLSVSPGERATLSCRASQSIGTNIHWYQQKPGQAPRLLIYYASESISGIPARFSGSGSGTEFTLTISSLQSEDFAVYYCQQSWSHPTTFGGGTKVEIK

SEQ ID NO: 195 - protein, 6 aa, synthetic construct
  REGION 1..6: note = Synthetic peptide
SGHRWE

SEQ ID NO: 196 - protein, 16 aa, synthetic construct
  REGION 1..16: note = Synthetic peptide
SIHYDGSTNYNPSVKG

SEQ ID NO: 197 - protein, 12 aa, synthetic construct
  REGION 1..12: note = Synthetic peptide
ATHYFGHWHFAV

SEQ ID NO: 198 - protein, 16 aa, synthetic construct
  REGION 1..16: note = Synthetic peptide
SIHYDHSTNYNPSVKG

SEQ ID NO: 199 - protein, 12 aa, synthetic construct
  REGION 1..12: note = Synthetic peptide
ATHYFGHHHFAV

SEQ ID NO: 200 - protein, 7 aa, synthetic construct
  REGION 1..7: note = Synthetic peptide
WGSYLRS

SEQ ID NO: 201 - protein, 9 aa, synthetic construct
  REGION 1..9: note = Synthetic peptide
QQNAEDPYT

SEQ ID NO: 202 - protein, 15 aa, synthetic construct
  REGION 1..15: note = Synthetic peptide
RASQSVDYHGDSYMN

SEQ ID NO: 203 - protein, 15 aa, synthetic construct
  REGION 1..15: note = Synthetic peptide
RASQSVDYDGDHYMN

SEQ ID NO: 204 - protein, 15 aa, synthetic construct
  REGION 1..15: note = Synthetic peptide
RASQSVDYHGDHYMN

SEQ ID NO: 205 - protein, 121 aa, synthetic construct
  REGION 1..121: note = Synthetic peptide
EVQLVESGGGLVQPGGSLRLSCAVSGYSITSGHRWEWIRQAPGKGLEWVASIHYDGSTNYNPSVKGRITISRDDSKNTFYLQMNSLRAEDTAVYYCARATHYFGHWHFAVWGQGTLVTVSS

SEQ ID NO: 206 - protein, 121 aa, synthetic construct
  REGION 1..121: note = Synthetic peptide
EVQLVESGGGLVQPGGSLRLSCAVSGYSITSGHRWEWIRQAPGKGLEWVASIHYDHSTNYNPSVKGRITISRDDSKNTFYLQMNSLRAEDTAVYYCARATHYFGHWHFAVWGQGTLVTVSS

SEQ ID NO: 207 - protein, 121 aa, synthetic construct
  REGION 1..121: note = Synthetic peptide
EVQLVESGGGLVQPGGSLRLSCAVSGYSITSGHRWEWIRQAPGKGLEWVASIHYDGSTNYNPSVKGRITISRDDSKNTFYLQMNSLRAEDTAVYYCARATHYFGHHHFAVWGQGTLVTVSS

SEQ ID NO: 208 - protein, 121 aa, synthetic construct
  REGION 1..121: note = Synthetic peptide
EVQLVESGGGLVQPGGSLRLSCAVSGYSITSGHRWEWIRQAPGKGLEWVASIHYDHSTNYNPSVKGRITISRDDSKNTFYLQMNSLRAEDTAVYYCARATHYFGHHHFAVWGQGTLVTVSS

SEQ ID NO: 209 - protein, 111 aa, synthetic construct
  REGION 1..111: note = Synthetic peptide
DIQLTQSPSSLSASVGDRVTITCRASQSVDYDGDSYMNWYQQKPGKAPKLLIEWGSYLRSGVPSRFSGSGSGTDFTLTISSLQPEDFATYYCQQNAEDPYTFGQGTKVEIK

SEQ ID NO: 210 - protein, 111 aa, synthetic construct
  REGION 1..111: note = Synthetic peptide
DIQLTQSPSSLSASVGDRVTITCRASQSVDYHGDSYMNWYQQKPGKAPKLLIEWGSYLRSGVPSRFSGSGSGTDFTLTISSLQPEDFATYYCQQNAEDPYTFGQGTKVEIK

SEQ ID NO: 211 - protein, 111 aa, synthetic construct
  REGION 1..111: note = Synthetic peptide
DIQLTQSPSSLSASVGDRVTITCRASQSVDYDGDHYMNWYQQKPGKAPKLLIEWGSYLRSGVPSRFSGSGSGTDFTLTISSLQPEDFATYYCQQNAEDPYTFGQGTKVEIK

SEQ ID NO: 212 - protein, 111 aa, synthetic construct
  REGION 1..111: note = Synthetic peptide
DIQLTQSPSSLSASVGDRVTITCRASQSVDYHGDHYMNWYQQKPGKAPKLLIEWGSYLRSGVPSRFSGSGSGTDFTLTISSLQPEDFATYYCQQNAEDPYTFGQGTKVEIK

SEQ ID NO: 213 - protein, 9 aa, synthetic construct
  REGION 1..9: note = Synthetic peptide
QQSDSHPTT

SEQ ID NO: 214 - protein, 9 aa, synthetic construct
  REGION 1..9: note = Synthetic peptide
HQSDSWPTT

SEQ ID NO: 215 - protein, 124 aa, synthetic construct
  REGION 1..124: note = Synthetic peptide
QLLESGGGLVQPGGSLRLSCAASGFTFSSYVMSWVRQAPGKGLEWVSSIYHDGSHTYYADFVKGRFTISRDNSKNTLYLQMNSLRAEDTAVYYCAKGTSYSGSYYYTDPFFGSWGQGTLVTVSS

SEQ ID NO: 216 - protein, 107 aa, synthetic construct
  REGION 1..107: note = Synthetic peptide
SSELTQDPAVSVALGQTVRITCQGDRLGSRYIYWYQQKPGQAPVLVIYDDDRRPSGIPDRFSGSSSGNTASLTITGAQAEDEADYYCQSADSSGNPVFGGGTQLTVL